(19) United States
(12) Patent Application Publication
(10) Pub. No.: US 2020/0276363 A1
(43) Pub. Date: Sep. 3, 2020

(54) AMINO ACID-BASED POLY(ESTER UREA) POLYMER MESH FOR HERNIA AND OTHER SOFT TISSUE APPLICATIONS

(71) Applicant: THE UNIVERSITY OF AKRON, Akron, OH (US)

(72) Inventors: Matthew BECKER, Chapel Hill, NC (US); Nathan Z. DREGER, Copley, OH (US); Chinnapatch TANTISUWANNO, Akron, OH (US); Claus SOENDERGAARD, West Lafayette, IN (US); Mary WADE, Laffayette, IN (US)

(21) Appl. No.: 16/765,995

(22) PCT Filed: Nov. 21, 2018
(86) PCT No.: PCT/US2018/062241

§ 371 Date: May 21, 2020

Related U.S. Application Data
(60) Provisional application No. 62/589,128, filed on Nov. 21, 2017.

(51) Int. Cl. A61L 31/06; A61L 31/00; A61L 31/14; A61L 31/12; B29D 28/00

(52) U.S. Cl. CPC A61L 31/06; A61L 31/005; B29K 2067/00; A61L 31/12; B29D 28/00; A61L 31/146

(57) ABSTRACT

In one or more embodiments, the present invention is directed to a implantable polymer mesh for use in hernia and other soft tissue repair made using amino acid based poly(ester urea) (PEU) polymers. In some embodiments, the implantable polymer mesh is made using linear or branched L-valine based PEUs and displays mechanical properties similar to poly(propylene) (PP), but with significantly less fibrous capsule formation. In some embodiments, the implantable polymer mesh is made using L-valine-co-L-phenylalanine PEUs. In some embodiments, the implantable polymer mesh is made using these PEUs in a composite with an extracellular matrix (ECM). In various embodiments, these amino acid-based PEU materials can be formed into implantable polymer mesh having a conventional size and shape by a wide variety of techniques including conventional compression molding, vacuum molding, blade coating, flow coating, and/or solvent casting.

### CROSS REFERENCE TO RELATED APPLICATIONS

This application claims the benefit of U.S. provisional patent application Ser. No. 62/589,128 entitled “Amino Acid-Based Poly(Ester Urea) Polymer Mesh For Hernia and Other Soft Tissue Applications,” filed Nov. 21, 2017, and incorporated herein by reference in its entirety.

### NAMES OF THE PARTIES TO A JOINT RESEARCH AGREEMENT

The present application stems from work done pursuant to a Joint Research Agreement between The University of Akron of Akron Ohio and Cook Medical Technologies, LLC of Bloomington, Ind.

### FIELD OF THE INVENTION

One or more embodiments of the present invention relates to a medical implant for soft tissue repair. In certain embodiments, the present invention relates to an implantable poly(ester urea) polymer mesh for use in hernia and other soft tissue repair.

### BACKGROUND OF THE INVENTION

Synthetic polymers have been used in medical devices for more than 50 years. Hernias are one medical malady that has utilized polymer devices to help aide in clinical outcome. A hernia arises from a structural defect in surrounding tissue or muscle. The location of the defect can vary across the body with the most common types occurring at the inner groin (inguinal) and the abdomen (ventral). In the 1800s, sutures were used to close the herniated tissue and unsurprisingly, recurrence rates were high. Polymer devices have since been utilized to help augment the structural defect which led to a significant drop in recurrence with some inguinal hernia rates being reported for less than 15% of cases. Rates vary greatly depending on multiple factors including hernia type, surgical complexity, and patient preexisting conditions. Despite the advancements in surgical techniques and better surgical success rates, much is left to be desired from a material standpoint.

Applications in tissue regeneration for hernia-mesh repair are of interest because of unmet needs from currently employed materials. Synthetic polymers currently being clinically utilized and explored include poly(propylene) (PP), polyesters (PES), lactones (PCL), lactides (PLA), polyvinylidene fluoride (PVDF), polyurethanes (PU), and various copolymers thereof. Other potential solutions from xyogeneic and allogeneic materials have also been explored. Regardless of the material chosen for study, there are several criteria that the material must address in order to meet the needs for a hernia injury: limited inflammatory immune response, provide strong reinforcement to affected area, promoting native tissue regeneration at the wound site, and degradation over time to prevent recurrence and patient discomfort.

Poly(propylene) (PP) mesh has been widely used to aid in the treatment of ventral hernias. PP mesh provides strong reinforcement to the affected area which has helped reduce the rate of recurrence from previous surgical methods. Despite vast improvements from previous surgical techniques, PP's rigidity promotes the deposition of rigid, fibrous scar-tissue which is foreign to the injury site and can lead to recurrence. On top of this, PP is also non-resorbable which leaves the implant permanently in the patient. Immediate recurrence prevention comes at the cost of long term comfort and structural integrity of the wound healing site.

Since PP was first introduced over 50 years ago small steps have been made to come up with alternative materials which address such problems. Homopolymers and copolymers consisting of PCL, PLA, and PGA all have improved degradation properties when compared to PP with mechanical properties that are comparable to that of PP. However, despite these materials' processability and improved degradation rates, the degradation byproducts can promote an undesired inflammatory response at the wound healing site. To mitigate these issues, use of xyogeneic and allogeneic materials has been attempted (porcine, human skin grafts, etc.). Extracellular matrix (ECM) materials have been shown to promote healing at the wound site with limited inflammatory response, however, the mechanical properties of these materials deteriorate rapidly in vivo, which ultimately leads to recurrence. ECM materials are also precluded as a permanent solution by patient dependent cost.

Poly(ester urea)s (PEU)s have become a novel material of interest for hernia repair as they have previously been shown to display tunable mechanical properties and degradation rates, and to elicit a limited inflammatory response in vivo. See, e.g., Yu, J.; Lin, F.; Becker, M. L. “Branched amino acid based poly(ester urea)s with tunable thermal and water uptake properties.” Macromolecules 2015 DOI: 10.1021/acs.macromol.5b00376. and Yu, J.; Lin, F.; Lin, P.; Gao, Y.; Becker, M. L. “Phenylalanine-based poly(ester urea): Synthesis, characterization, and in vitro degradation.” Macromolecules 2014 DOI: 10.1021/ma401752b, the disclosures of which are incorporated herein by reference. α-Amino acid based PEUs tunable properties is based on monomer diol chain length, amino acid selected, and degree of branching (longer diol chain length leads to greater chain flexibility and a lower elastic modulus). Degradation byproducts of α-amino acid based PEUs have been previously shown to have no observable local acidic inflammatory response.

What is needed in the art are resorbable materials for use in mesh for hernia repair and other soft tissue applications that bridge the gap between the mechanically competent PP and the cellular active ECM and simultaneously possesses tunable degradation rates, sufficient mechanical properties throughout the healing process, and elicit a limited foreign body response.

### SUMMARY OF THE INVENTION

In one or more embodiments, the present invention is directed to an implantable polymer mesh for use in hernia and other soft tissue repair made using amino acid based poly(ester urea) polymers that have tunable degradation rates, sufficient mechanical properties throughout the healing process, and elicit a limited foreign body response. In some embodiments, the implantable polymer mesh of the present invention is made using linear and/or branched L-valine based PEUs that display mechanical properties similar to poly(propylene) (PP), but with significantly less fibrous capsule formation at the wound healing site. In some other embodiments, the implantable polymer mesh of the present invention is made using L-valine-co-L-phenylalanine PEUs, either alone or as a composite with an extracellular matrix (ECM). In various embodiments, these amino acid-based PEU materials can be formed into implantable polymer mesh having a conventional size and shape for use in hernia and other soft tissue repair by a wide variety of techniques including conventional compression molding, vacuum molding, blade coating, flow coating, and/or solvent casting.

In a first aspect, the present invention is directed to a polymer mesh for soft tissue repair comprising an amino acid-based poly(ester urea) polymer. In one or more of these embodiments, the amino acid-based poly(ester urea) polymer forming the polymer mesh has a number average molecular weight (Mₙ) of from about 10,000 g/mol to about 500,000 g/mol, as measured by size exclusion chromatography (SEC). In one or more embodiments, the polymer mesh for soft tissue repair of the present invention includes any one or more of the above referenced embodiments of the first aspect of the present invention wherein the amino acid-based poly(ester urea) polymer has a weight average molecular weight (M_{w}) of from about 10,000 g/mol to about 250,000 g/mol, as measured by size exclusion chromatography (SEC). In one or more embodiments, the polymer mesh for soft tissue repair of the present invention includes any one or more of the above referenced embodiments of the first aspect of the present invention wherein the amino acid-based poly(ester urea) polymer has a molecular mass distribution of (Ðₘ) of from about 1.1 to about 3.0 as measured by size exclusion chromatography (SEC).

In one or more embodiments, the polymer mesh for soft tissue repair of the present invention includes any one or more of the above referenced embodiments of the first aspect of the present invention wherein the amino acid-based poly(ester urea) polymer has a glass transition temperature (T_{g}) of from about 28° C. to about 57° C. as measured by differential scanning calorimetry. In one or more embodiments, the polymer mesh for soft tissue repair of the present invention includes any one or more of the above referenced embodiments of the first aspect of the present invention wherein the amino acid-based poly(ester urea) polymer has a degradation temperature (T_{d}) of from about 200° C. to about 350° C. as measured by thermogravimetric analysis.

In one or more embodiments, the polymer mesh for soft tissue repair of the present invention includes any one or more of the above referenced embodiments of the first aspect of the present invention wherein the amino acid based PEU polymer has a Young's modulus of from about 10 MPa to about 500 MPa as measured by uniaxial tensile testing. In one or more embodiments, the polymer mesh for soft tissue repair of the present invention includes any one or more of the above referenced embodiments of the first aspect of the present invention wherein the amino acid based PEU polymer has a yield stress (σ_{y}) of from about 1 MPa to about 100 MPa as measured by uniaxial tensile testing. In one or more embodiments, the polymer mesh for soft tissue repair of the present invention includes any one or more of the above referenced embodiments of the first aspect of the present invention wherein the amino acid based PEU polymer has a yield strain (ε_{y}) of from about 2% to about 50% as measured by uniaxial tensile testing.

In one or more embodiments, the polymer mesh for soft tissue repair of the present invention includes any one or more of the above referenced embodiments of the first aspect of the present invention wherein the amino acid based PEU polymer has a force at break from about 30 N to about 300 N as measured by burst testing at a constant rate of traverse of 25.4 mm/min. In one or more embodiments, the polymer mesh for soft tissue repair of the present invention includes any one or more of the above referenced embodiments of the first aspect of the present invention wherein the amino acid based PEU polymer has an extension at break from about 0.5 cm to about 5 cm as measured by burst testing at a constant rate of traverse of 25.4 mm/min.

In one or more embodiments, the polymer mesh for soft tissue repair of the present invention includes any one or more of the above referenced embodiments of the first aspect of the present invention wherein the amino acid based PEU polymer has a relative stiffness from about 25 N/cm to about 200 N/cm as measured by burst testing at a constant rate of traverse of 25.4 mm/min. In one or more embodiments, the polymer mesh for soft tissue repair of the present invention includes any one or more of the above referenced embodiments of the first aspect of the present invention wherein the amino acid based PEU polymer has a water uptake from about 0 mass % to about 45 mass % as measured by phosphine buffered saline solution (PBS) swelling/water uptake measurements.

In a second aspect, the present invention is directed to a polymer mesh for soft tissue repair wherein the amino acid-based poly(ester urea) polymer comprises the residue of two or more amino acid based polyester monomers separated by urea bonds, wherein the one or more amino acid based polyester monomer residues each comprise the residues of two amino acids separated by from each other by from about 2 to about 20 carbon atoms. In one or more embodiments, the polymer mesh for soft tissue repair of the present invention includes any one or more of the above referenced embodiments of the second aspect of the present invention wherein one or more of the two or more amino acid based polyester monomers are branched. In one or more embodiments, the polymer mesh for soft tissue repair of the present invention includes any one or more of the above referenced embodiments of the second aspect of the present invention wherein each of the two amino acids are selected from the group consisting of L-valine, L-Leucine, L-Isoleucine, L-serine, L-alanine, L-glycine, L-aspartic acid, L-asparagine, L-arginine, L-phenylalanine, L-methionine, benzyl protected L-tyrosine, and combinations thereof.

In one or more embodiments, the polymer mesh for soft tissue repair of the present invention includes any one or more of the above referenced embodiments of the second aspect of the present invention wherein the residues of the one or more amino acid based polyester monomer residues comprise two valine residues separated by from about 2 to about 20 carbon atoms. In one or more embodiments, the polymer mesh for soft tissue repair of the present invention includes any one or more of the above referenced embodiments of the second aspect of the present invention wherein the one or more amino acid based polyester monomer residues comprise two phenylalanine residues separated by from about 2 to about 20 carbon atoms.

In one or more embodiments, the polymer mesh for soft tissue repair of the present invention includes any one or more of the above referenced embodiments of the second aspect of the present invention wherein the amino acid-based poly(ester urea) polymer has the formula:

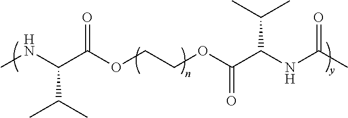

where n is an integer from 1 and 12; and y is an integer from about 10 to about 1000.

In one or more embodiments, the polymer mesh for soft tissue repair of the present invention includes any one or more of the above referenced embodiments of the second aspect of the present invention wherein the amino acid-based poly(ester urea) polymer has a number average molecular weight (Mₙ) of from about 10,000 g/mol to about 500,000 g/mol, as measured by size exclusion chromatography (SEC). In one or more embodiments, the polymer mesh for soft tissue repair of the present invention includes any one or more of the above referenced embodiments of the second aspect of the present invention wherein the amino acid-based poly(ester urea) polymer has a weight average molecular weight (M_{w}) of from about 10,000 g/mol to about 250,000 g/mol, as measured by size exclusion chromatography (SEC). In one or more embodiments, the polymer mesh for soft tissue repair of the present invention includes any one or more of the above referenced embodiments of the second aspect of the present invention wherein the amino acid-based poly(ester urea) polymer has a molecular mass distribution of (Ðₘ) of from about 1.1 to about 3.0 as measured by size exclusion chromatography (SEC).

In one or more embodiments, the polymer mesh for soft tissue repair of the present invention includes any one or more of the above referenced embodiments of the second aspect of the present invention wherein the amino acid-based poly(ester urea) polymer has a glass transition temperature (T_{g}) of from about 28° C. to about 57° C. as measured by differential scanning calorimetry. In one or more embodiments, the polymer mesh for soft tissue repair of the present invention includes any one or more of the above referenced embodiments of the second aspect of the present invention wherein the amino acid-based poly(ester urea) polymer has a degradation temperature 1, 9, or (T_{d}) of from about 200° C. to about 350° C. as measured by thermogravimetric analysis.

In one or more embodiments, the polymer mesh for soft tissue repair of the present invention includes any one or more of the above referenced embodiments of the second aspect of the present invention wherein the amino acid-based poly(ester urea) polymer has a Young's modulus of from about 10 MPa to about 500 MPa as measured by uniaxial tensile testing. In one or more embodiments, the polymer mesh for soft tissue repair of the present invention includes any one or more of the above referenced embodiments of the second aspect of the present invention wherein the amino acid-based poly(ester urea) polymer has having a yield stress (σ_{y}) of from about 1 MPa to about 100 MPa as measured by uniaxial tensile testing. In one or more embodiments, the polymer mesh for soft tissue repair of the present invention includes any one or more of the above referenced embodiments of the second aspect of the present invention wherein the amino acid-based poly(ester urea) polymer has a yield strain (ε_{y}) of from about 2% to about 50% as measured by uniaxial tensile testing.

In one or more embodiments, the polymer mesh for soft tissue repair of the present invention includes any one or more of the above referenced embodiments of the second aspect of the present invention wherein the amino acid-based poly(ester urea) polymer has a force at break from about 30 N to about 300 N as measured by burst testing at a constant rate of traverse of 25.4 mm/min. In one or more embodiments, the polymer mesh for soft tissue repair of the present invention includes any one or more of the above referenced embodiments of the second aspect of the present invention wherein the amino acid-based poly(ester urea) polymer has an extension at break from about 0.5 cm to about 5 cm as measured by burst testing at a constant rate of traverse of 25.4 mm/min. In one or more embodiments, the polymer mesh for soft tissue repair of the present invention includes any one or more of the above referenced embodiments of the second aspect of the present invention wherein the amino acid-based poly(ester urea) polymer has a relative stiffness from about 25 N/cm to about 200 N/cm as measured by burst testing at a constant rate of traverse of 25.4 mm/min.

In one or more embodiments, the polymer mesh for soft tissue repair of the present invention includes any one or more of the above referenced embodiments of the second aspect of the present invention wherein the amino acid-based poly(ester urea) polymer has a water uptake from about 0 mass % to about 45 mass % as measured by phosphine buffered saline solution (PBS) swelling/water uptake measurements

In a third aspect, the present invention is directed to a polymer mesh for soft tissue repair as described above wherein the amino acid-based poly(ester urea) polymer is a copolymer comprising a first type of amino acid based polyester monomer residue and a second type amino acid based polyester monomer residue separated by urea bonds, wherein the first type of amino acid based polyester monomer residue and the second type of amino acid based polyester monomer residue have different chemical structures.

In some of these embodiments, the first type of amino acid based polyester monomer residue comprises two amino acid residues separated by from about 2 to about 20 carbon atoms. In one or more embodiments, the polymer mesh for soft tissue repair of the present invention includes any one or more of the above referenced embodiments of the third aspect of the present invention wherein each of the two amino acids in the first type of amino acid based polyester monomer are selected from the group consisting of L-valine, L-leucine, L-isoleucine, L-serine, L-alanine, L-glycine, L-aspartic acid, L-asparagine, L-arginine, L-phenylalanine, L-methionine, benzyl protected L-tyrosine, and combinations thereof. In one or more embodiments, the polymer mesh for soft tissue repair of the present invention includes any one or more of the above referenced embodiments of the third aspect of the present invention wherein the first type of amino acid based polyester monomer residue comprises two valine residues separated by from about 2 to about 20 carbon atoms. In one or more embodiments, the polymer mesh for soft tissue repair of the present invention includes any one or more of the above referenced embodiments of the third aspect of the present invention wherein the first type of amino acid based polyester monomer residue comprises three or more valine residues, wherein each of the three or more valine residues is separated from the other valine residues by from about 2 to about 20 carbon atoms.

In one or more embodiments, the polymer mesh for soft tissue repair of the present invention includes any one or more of the above referenced embodiments of the third aspect of the present invention wherein the second type of amino acid based polyester monomer residue comprises two amino acid residues separated by from about 2 to about 20 carbon atoms. In one or more embodiments, the polymer mesh for soft tissue repair of the present invention includes any one or more of the above referenced embodiments of the third aspect of the present invention wherein each of the two amino acid residues in the second type of amino acid based polyester monomer is selected from the group consisting of L-valine, L-leucine, L-isoleucine, L-serine, L-alanine, L-glycine, L-aspartic acid, L-asparagine, L-arginine, L-phenylalanine, L-methionine, benzyl protected L-tyrosine, and combinations thereof. In one or more embodiments, the polymer mesh for soft tissue repair of the present invention includes any one or more of the above referenced embodiments of the third aspect of the present invention wherein the second type of amino acid based polyester monomer comprises two phenylalanine residues separated by from about 2 to about 20 carbon atoms. In one or more embodiments, the polymer mesh for soft tissue repair of the present invention includes any one or more of the above referenced embodiments of the third aspect of the present invention wherein the molar ratio of the first amino acid based polyester monomer residue and a second amino acid based polyester monomer residue is from about 1:19 to about 19:1.

In one or more embodiments, the polymer mesh for soft tissue repair of the present invention includes any one or more of the above referenced embodiments of the third aspect of the present invention wherein the first type of amino acid based polyester monomer residue comprises two valine residues separated by from about 2 to about 20 carbon atoms and the second type of amino acid based polyester monomer residue comprises two phenylalanine residues separated by from about 2 to about 20 carbon atoms. In one or more embodiments, the polymer mesh for soft tissue repair of the present invention includes any one or more of the above referenced embodiments of the third aspect of the present invention wherein the second type of amino acid based polyester monomer residue comprises from about 5 mole percent to about 30 mole percent of the amino acid-based poly(ester urea) polymer.

In one or more embodiments, the polymer mesh for soft tissue repair of the present invention includes any one or more of the above referenced embodiments of the third aspect of the present invention wherein the amino acid-based poly(ester urea) polymer has the formula:

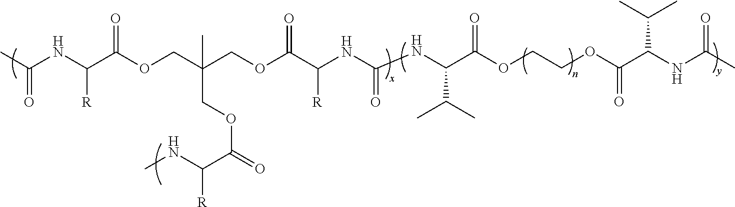

where n is an integer from 1 and 12; x is a mole fraction from about 0.001 to about 0.100; y is a mole fraction from about 0.900 to about 0.999; and each R is selected from the group consisting —CH₃, —(CH₂)₃NHC(NH₂)C═NH, —CH₂CONH₂, —CH₂COOH, —CH₂SH, —(CH₂)₂COOH, —(CH₂)₂CONH₂, —NH₂, —CH₂C=C H—N═CH—NH, —CH(CH₃)CH₂CH₃, —CH₂CH—(CH₃)₂, —(CH₂)₄NH₂, —(CH₂)₂SCH₃, —CH₂Ph, —CH₂OH, —CH(OH)CH₃, —CH, —C═CH—NH-Ph, —CH₂-Ph-OH, or —CH(CH₃)₂.

In one or more embodiments, the polymer mesh for soft tissue repair of the present invention includes any one or more of the above referenced embodiments of the third aspect of the present invention wherein the amino acid-based poly(ester urea) polymer has the formula:

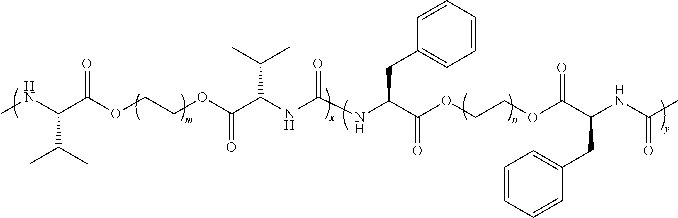

where n and m can be between 1 and 12; x is a mole fraction from about 0.05 to about 0.95; y is a mole fraction from about 0.95 to about 0.05. In one or more of these embodiments, the polymer mesh for soft tissue repair of the present invention includes any one or more of the above referenced embodiments of the third aspect of the present invention wherein y is a mole fraction from about 0.05 to about 0.30.

In one or more embodiments, the polymer mesh for soft tissue repair of the present invention includes any one or more of the above referenced embodiments of the third aspect of the present invention wherein the amino acid-based poly(ester urea) polymer is degradable within the body of a patient.

In one or more embodiments, the polymer mesh for soft tissue repair of the present invention includes any one or more of the above referenced embodiments of the third aspect of the present invention further comprising an extracellular matrix (ECM). In one or more embodiments, the polymer mesh for soft tissue repair of the present invention includes any one or more of the above referenced embodiments of the third aspect of the present invention wherein the extracellular matrix comprises 2.0 1-1 LL SIS-ECM, 2.0 4-LL SIS ECM, Blanket 2 LVP SIS ECM, or Blanket 4 LVP SIS ECM.

In one or more embodiments, the polymer mesh for soft tissue repair of the present invention includes any one or more of the above referenced embodiments of the third aspect of the present invention the amino acid-based poly(ester urea) polymer has a number average molecular weight (Mₙ) of from about 10,000 g/mol to about 500,000 g/mol, as measured by size exclusion chromatography (SEC). In one or more embodiments, the polymer mesh for soft tissue repair of the present invention includes any one or more of the above referenced embodiments of the third aspect of the present invention wherein the amino acid-based poly(ester urea) polymer has a weight average molecular weight (M_{w}) of from about 10,000 g/mol to about 250,000 g/mol, as measured by size exclusion chromatography (SEC). In one or more embodiments, the polymer mesh for soft tissue repair of the present invention includes any one or more of the above referenced embodiments of the third aspect of the present invention wherein the amino acid-based poly(ester urea) polymer has a molecular mass distribution of (Ðₘ) of from about 1.1 to about 3.0 as measured by size exclusion chromatography (SEC).

In one or more embodiments, the polymer mesh for soft tissue repair of the present invention includes any one or more of the above referenced embodiments of the third aspect of the present invention wherein the amino acid-based poly(ester urea) polymer has a glass transition temperature (T_{g}) of from about 28° C. to about 57° C. as measured by differential scanning calorimetry. In one or more embodiments, the polymer mesh for soft tissue repair of the present invention includes any one or more of the above referenced embodiments of the third aspect of the present invention wherein the amino acid-based poly(ester urea) polymer has a degradation temperature (T_{d}) of from about 200° C. to about 350° C., as measured by thermogravimetric analysis.

In one or more embodiments, the polymer mesh for soft tissue repair of the present invention includes any one or more of the above referenced embodiments of the third aspect of the present invention wherein the amino acid-based poly(ester urea) polymer has a Young's modulus of from about 10 MPa to about 500 MPa as measured by uniaxial tensile testing. In one or more embodiments, the polymer mesh for soft tissue repair of the present invention includes any one or more of the above referenced embodiments of the third aspect of the present invention wherein the amino acid-based poly(ester urea) polymer has a yield stress (σ_{y}) of from about 2 MPa to about 100 MPa as measured by uniaxial tensile testing. In one or more embodiments, the polymer mesh for soft tissue repair of the present invention includes any one or more of the above referenced embodiments of the third aspect of the present invention wherein the amino acid-based poly(ester urea) polymer has a yield strain (ε_{y}) of from about 1% to about 50% as measured by uniaxial tensile testing.

In one or more embodiments, the polymer mesh for soft tissue repair of the present invention includes any one or more of the above referenced embodiments of the third aspect of the present invention wherein the amino acid-based poly(ester urea) polymer has a force at break from about 50 N to about 500 N as measured by burst testing at a constant rate of traverse of 25.4 mm/min. In one or more embodiments, the polymer mesh for soft tissue repair of the present invention includes any one or more of the above referenced embodiments of the third aspect of the present invention wherein the amino acid-based poly(ester urea) polymer has an extension at break from about 0.5 cm to about 5 cm as measured by burst testing at a constant rate of traverse of 25.4 mm/min. In one or more embodiments, the polymer mesh for soft tissue repair of the present invention includes any one or more of the above referenced embodiments of the third aspect of the present invention wherein the amino acid-based poly(ester urea) polymer has a relative stiffness from about 25 N/cm to about 200 N/cm as measured by burst testing at a constant rate of traverse of 25.4 mm/min.

In one or more embodiments, the polymer mesh for soft tissue repair of the present invention includes any one or more of the above referenced embodiments of the third aspect of the present invention wherein the amino acid-based poly(ester urea) polymer has a water uptake from about 0 mass % to about 45 mass % as measured by phosphine buffered saline solution (PBS) swelling/water uptake testing

In one or more embodiments, the polymer mesh for soft tissue repair of the present invention includes any one or more of the above referenced embodiments of the third aspect of the present invention wherein the polymer mesh is formed by compression molding, blade coating, or vacuum molding.

In one or more embodiments, the polymer mesh for soft tissue repair of the present invention includes any one or more of the above referenced embodiments of the present invention wherein the polymer mesh elicits less inflammatory response than polymer mesh formed from polypropylene, as measured by hematoxylin and eosin histological fibrous capsule measurements. In one or more embodiments, the polymer mesh for soft tissue repair of the present invention includes any one or more of the above referenced embodiments of the present invention wherein the polymer mesh elicits less fibrous capsule formation than polymer mesh for soft tissue repair formed from polypropylene, as measured by hematoxylin and eosin histological fibrous capsule measurements.

In a fourth aspect, the present invention is directed to a method of forming the polymer mesh for soft tissue repair of described above comprising: forming an amino acid-based poly(ester urea) polymer comprising the residue of two or more amino acid based polyester monomers, wherein the one or more amino acid based polyester monomer residues each comprise two amino acid residues separated by from about 2 to about 20 carbon atoms and the two or more amino acid based polyester monomers are separated by urea bonds; and forming the amino acid-based polymer into a 3-dimensional mesh.

In some of these embodiments, the step of forming an amino acid-based poly(ester urea) polymer further comprises: preparing the acid salts of one or more amino acid based polyester monomers, wherein each of the one or more amino acid based polyester monomers comprises the residues of two amino-acids separated by from about 2 to about 20 carbon atoms; dissolving the acid salts of one or more amino acid based polyester monomers and a deprotecting base, preferably sodium carbonate, in a suitable solvent; preparing a solution containing a urea bond forming compound; and adding the urea bond forming compound containing solution to the amino acid based polyester monomers solution form the amino acid-based poly(ester urea) polymer.

In one or more embodiments, the method of forming polymer mesh for soft tissue repair of the present invention includes any one or more of the above referenced embodiments of the fourth aspect of the present invention wherein the one or more amino acid based polyester monomers comprises the residues of two valine molecules separated by from about 2 to about 20 carbon atoms. In one or more embodiments, the method of forming polymer mesh for soft tissue repair of the present invention includes any one or more of the above referenced embodiments of the fourth aspect of the present invention wherein the one or more amino acid based polyester monomers comprises the residues of two phenylalanine molecules separated by from about 2 to about 20 carbon atoms.

In one or more embodiments, the method of forming polymer mesh for soft tissue repair of the present invention includes any one or more of the above referenced embodiments of the fourth aspect of the present invention wherein the solution containing a urea bond forming compound comprises phosgene, diphosgene or triphosgene. In one or more embodiments, the method of forming polymer mesh for soft tissue repair of the present invention includes any one or more of the above referenced embodiments of the fourth aspect of the present invention wherein the solution containing a urea bond forming compound comprises triphosgene dissolved in chloroform.

In one or more embodiments, the method of forming polymer mesh for soft tissue repair of the present invention includes any one or more of the above referenced embodiments of the fourth aspect of the present invention wherein at least of the one or more amino acid based polyester monomers is a branched amino acid based polyester monomer. In one or more embodiments, the method of forming polymer mesh for soft tissue repair of the present invention includes any one or more of the above referenced embodiments of the fourth aspect of the present invention wherein the amino acid based polyester monomer is selected from the group consisting of tri-o-benzyl-L-tyrosine-1,1,1-trimethylethane, di-p-toluenesulfonic acid salts of bis(L-phenylalanine)-ethane 1,2-diester, di-p-toluenesulfonic acid salts of bis(L-phenylalanine)-butane 1,4-diester, di-p-toluenesulfonic acid salts of bis(L-phenylalanine)-hexane 1,6-diester, di-p-toluenesulfonic acid salts of bis(L-phenylalanine)-octane 1,8-diester, di-p-toluenesulfonic acid salts of bis(L-phenylalanine)-decane 1,10-diester, di-p-toluenesulfonic acid salts of bis(L-phenylalanine)-dodecane 1,12-diester, and di-p-toluenesulfonic acid salts of bis(L-phenylalanine)-tetradecane 1,14-diester, di-p-toluenesulfonic acid salts of bis(L-valine)-ethane 1,2-diester, di-p-toluenesulfonic acid salts of bis(L-valine)-butane 1,4-diester, di-p-toluenesulfonic acid salts of bis(L-valine)-hexane 1,6-diester, di-p-toluenesulfonic acid salts of bis(L-valine)-octane 1,8-diester, di-p-toluenesulfonic acid salts of bis(L-valine)-decane 1,10-diester, di-p-toluenesulfonic acid salts of bis(L-valine)-dodecane 1,12-diester, and di-p-toluenesulfonic acid salts of bis(L-valine)-tetradecane 1,14-diester monomer, and combinations thereof.

In one or more embodiments, the method of forming polymer mesh for soft tissue repair of the present invention includes any one or more of the above referenced embodiments of the fourth aspect of the present invention wherein the step of forming the amino acid-based polymer into a 3-dimensional mesh is performed by compression molding, vacuum molding, blade coating, flow coating, electrospinning or solvent casting.

In one or more embodiments, the method of forming polymer mesh for soft tissue repair of the present invention includes any one or more of the above referenced embodiments of the fourth aspect of the present invention wherein the step of forming the amino acid-based polymer into a 3-dimensional mesh comprises: pulverizing the amino acid-based poly(ester urea) polymer to a powder in a grinder; preparing a mold defining the desired shape of a 3-dimensional mesh; the mold being configured for use in a vacuum compression molding device; placing the amino acid-based poly(ester urea) polymer powder into the mold; heating the amino acid-based poly(ester urea) polymer powder in the mold to a temperature above its melting temperature (Tₘ) and below its degradation temperature (T_{d}) to melt the amino acid-based poly(ester urea) polymer powder; compressing the amino acid-based poly(ester urea) polymer in the mold with a force of from about 20 MPa to about 200 MPa; cooling the amino acid-based poly(ester urea) polymer to ambient temperature to provide the 3-dimensional mesh.

In a fifth aspect, the present invention is directed to a method of forming the polymer mesh for soft tissue repair described above comprising: dissolving amino acid-based poly(ester urea) polymer in to a suitable solvent or solvent solution; securing extracellular matrix (ECM) to a substrate to form a ECM/substrate combination that is configured for use in a blade coating, flow coating, or solvent casting device; feeding the amino acid-based poly(ester urea) polymer solution into a solution well that is configured for use in a blade coating, flow coating, or solvent casting device; securing the solvent well to a blade coating, flow coating, or solvent casting device and moving ECM/substrate combination through the blade coating, flow coating, or solvent casting device at a velocity from about 0 cm/s to about 200 cm/s to apply the amino acid-based poly(ester urea) polymer to the ECM/substrate combination with the ECM acting as the substrate for the amino acid-based poly(ester urea) polymer; removing the solvent from the poly(ester urea) polymer coated extracellular ECM/substrate combination by drying at a temperature of from about 20° C. to about 35° C. for a period of from about 1 hour to about 24 hours; placing the poly(ester urea) polymer coated extracellular ECM/substrate combination under a vacuum pressure of from about 5 mm/Hg to about 25 mm/Hg for from about 1 hour to about 24 hours to remove any residual solvent; and removing the PEU/ECM composite from the substrate to provide the PEU/ECM composite mesh.

### BRIEF DESCRIPTION OF THE DRAWINGS

For a more complete understanding of the features and advantages of the present invention, reference is now made to the detailed description of the invention along with the accompanying figures in which:

FIG. 1 is a ¹H-NMR overlays showing the ¹H-NMR spectra of linear monomers 1-VAL-8, 1-VAL-10, and 1-VAL-12. The peaks “b” indicated in the highlighted regions denote the methylene peaks for each of the three monomers.

FIG. 2 is a ¹H-NMR of the Triol-TYR is shown (top spectrum), and successful boc-deprotection (bottom spectrum) was identified by the disappearance of the methylene peak at 1.28 ppm and the appearance of the broad amine proton peak between 8.72-8.78 ppm, both of which are highlighted.

FIG. 3 is a ¹H-NMR overlay of spectra of linear poly(ester urea)s poly(1-VAL-8), poly(1-VAL-10), and poly(1-VAL-12). The highlighted peaks “b” indicate the methylene peaks from the diol chain lengths for each of the three polymers.

FIG. 4 is a ¹H-NMR spectra of branched poly[(1-VAL-8)_{0.98}-co-(Triol-TYR)_{0.02}].

FIG. 5 is a ¹H-NMR spectra poly[(1-VAL-10)_{0.98}-co-(Triol-TYR)_{0.02}]. The degree of branching was determined by integration of the six methylene protons denoted “e” from the Triol-TYR monomer and comparing them to the twelve methyl L-valine protons denoted “n” from the linear monomers.

FIGS. 6A-B are graphs showing the results of thermal gravimetric analysis (TGA) of the linear (FIG. 6A) and branched polymers (FIG. 6B) according to one or more embodiments of the present invention showing the degradation temperatures for these materials well above the temperature required for compression molding (100° C.)

FIG. 7 is a graph showing the results of differential scanning calorimetry (DSC) analysis of the linear and branched polymers show that the glass transition temperatures for these materials are near physiological conditions. The glass transition temperatures are significantly lower than the degradation temperature which allowed for these materials to be processed.

FIGS. 8A-E are size-exclusion chromatographs for PEUs according to one or more embodiments of the present invention comparing the initial molecular weights, post EtO sterilization, and the 2 and 3 month time points in vivo for (FIG. 8A) P(1-VAL-8), (FIG. 8B) P(1-VAL-10), (FIG. 8C) P(1-VAL-12), (FIG. 8D) 2% branched P(1-VAL-8), and (FIG. 8E) 2% branched P(1-VAL-10).

FIGS. 9A-F are scanning electron microscopy (SEM) images showing variations in the surface morphology for: P(1-VAL-8) (FIG. 9A), p(1-VAL-10) (FIG. 9B), p(1-VAL-12) (FIG. 9C), PP (FIG. 9D), 2% branched p(1-VAL-8) (FIG. 9E), and 2% branched p(1-VAL-10) (FIG. 9F). The images were captured at 750× magnification and scale bars indicate 10 am.

FIGS. 10A-F are graphs showing yield stress (σ_{y}) for polymers according to one or more embodiments of the present invention measured at the yield point. (FIG. 10A) P(1-VAL-8) σ_{y} values were assessed over 3 month time points (* indicates p value <0.05 between Initial and 2 month, and between Initial and 3 month samples. ** indicates p value <0.05 between Post EtO and 2 month, and between Post EtO and 3 month samples, n=4-6 samples). (FIG. 10B) P(1-VAL-10) σ_{y} values were assessed over 3 month time points (* indicates p value <0.05 between Initial and 2 month samples. ** indicates p value <0.05 between Post EtO and 2 month, and between Post EtO and 3 month samples, n=4-6). (FIG. 10C) P(1-VAL-12) σ_{y} values were assessed over 3 month time points (* indicates p value <0.05 between Initial and Post EtO, Initial and 2 month, and Initial and 3 month samples. ** indicates p value <0.05 between Post EtO and 2 month, and Post EtO and 3 month samples, n=4-6 samples). (FIG. 10D) PP σ_{y} values were assessed over 3 month time points and no significant difference was observed (p value <0.05 for n=4-6 samples). (FIG. 10E) 2% branched P(1-VAL-8) σ_{y} values were assessed over 3 month time points (* indicates p value <0.05 between Initial and Post EtO, between Initial and 2 month, and between Initial and 3 month samples. ** indicates p value <0.05 between Post EtO and 2 month, and between Post EtO and 3 month samples. *** indicates p value <0.05 between 2 month and 3 month samples, n=4-6 samples). (FIG. 10F) 2% branched P(1-VAL-10) σ_{y} values were assessed through 3 month time points (* indicates p value <0.05 between Initial and 2 Month, and between Initial and 3 Month samples, n=4-6 samples).

FIGS. 11A-F are graphs showing yield strain (e) for polymers according to one or more embodiments of the present invention measured at the yield point. (FIG. 11A). P(1-VAL-8) ε^{y} values were assessed through 3 month time points and no significant difference was observed (p value <0.05 for n=4-6 samples). (FIG. 11B) P(1-VAL-10) ε_{y} values were assessed through 3 month time points (* indicates p value <0.05 between Initial and 3 month samples. ** indicates p value <0.05 between 2 month and 3 month samples, n=4-6 samples). (FIG. 11C). P(1-VAL-12)) e values were assessed through 3 month time points and no significant difference was observed (p value <0.05 for n=4-6 samples). (FIG. 11D) ε_{y} values were assessed through 3 month time points (* indicates p value <0.05 between Initial and 2 month samples, n=4-6 samples). (FIG. 11D)) 2% branched P(1-VAL-8)_{y} values were assessed through 3 month time points (* indicates p value <0.05 between Initial and 3 month samples. ** indicates p value <0.05 between Post EtO and 3 Month samples, n=4-6). (FIG. 11F) 2% branched P(1-VAL-10) ε_{y} values were assessed through 3 month time points (* indicates p value <0.05 between Initial and 2 month, and between Initial and 3 month samples, n=4-6 samples).

FIGS. 12A-F are stress-strain curves for (FIG. 12A) P(1-VAL-8), (FIG. 12B) P(1-VAL-10), (FIG. 12C) P(1-VAL-12), (FIG. 12D) PP, (FIG. 12E) 2% branched P(1-VAL-8), and (FIG. 12F) 2% branched P(1-VAL-10) obtained from a tensile test performed at 25° C. at a rate of 25.4 mm/min. All mechanical data were extrapolated from the curves which represent an average of 4-6 samples.

FIGS. 13A-F are a series of graphs showing Young's modulus for implanted materials extrapolated at each time-point through linear regression with R²=0.98. (FIG. 13A) P(1-VAL-8) moduli values were assessed over 3 month time points (* indicates p value <0.05 between Initial and Post EtO, between Initial and 2 month, and Initial and 3 month samples. ** indicates p value <0.05 between Post EtO and 2 month, and between Post EtO and 3 month samples, n=4-6 samples). (FIG. 13B) P(1-VAL-10) moduli values were assessed over 3 month time points (* indicates p value <0.05 between Initial and 2 month, and between Initial and 3 month samples. ** indicates p value <0.05 between Post EtO and 2 month, and between Post EtO and 3 month, n=4-6 samples). (FIG. 13C) P(1-VAL-12) moduli values were assessed over 3 month time points (* indicates p value <0.05 between Initial and Post EtO, and between Initial and 3 month. ** indicates p value <0.05 between Post EtO and 2 month, and between Post EtO and 3 month, n=4-6 samples). (FIG. 13D) PP moduli values were assessed over 3 month time points (* indicates p value <0.05 between Initial and 2 Month samples. ** indicates p value <0.05 between 2 month and 3 month samples, n=4-6 samples). (FIG. 13E) 2% branched P(1-VAL-8) moduli values were assessed over 3 month time points (* indicates p value <0.05 between Initial and 2 month, and between Initial and 3 month samples. ** indicates p value <0.05 between Post EtO and 2 month, and between Post EtO and 3 month samples, n=4-6 samples). (FIG. 13F) 2% branched P(1-VAL-10) moduli values were assessed over 3 month time points (* indicates p value <0.05 between Initial and 2 month, and between Initial and 3 month samples. ** indicates p value <0.05 between Post EtO and 2 month, and Post EtO and 3 month samples, n=4-6 samples).

FIGS. 14A-F are histology images of (FIG. 14A) P(1-VAL-8), (FIG. 14B) P(1-VAL-10), (FIG. 14C) P(1-VAL-12), (FIG. 14D) PP, (FIG. 14E) 2% branched P(1-VAL-8), and (FIG. 14F) 2% branched P(1-VAL-10) showing the cross-sectional area of polymer and surrounding tissue, which was stained with hematoxylin and eosin and subsequently imaged. All images are at 20× magnification with scale bars being equal to 1 mm.

FIGS. 15A-B are graphs showing capsule thickness values for 2 month samples (FIG. 15A) were measured to assess inflammatory response (* indicates p value <0.01 between P(1-VAL-8) and P(1-VAL-12) and between P(1-VAL-8) and 2% branched P(1-VAL-8) samples. ** indicates p value <0.01 between P(1-VAL-10) and P(1-VAL-12), and between P(1-VAL-10) and 2% branched P(1-VAL-8) samples. *** indicates p value <0.01 between P(1-VAL-12) and PP, and between P(1-VAL-12) and 2% branched P(1-VAL-10) samples. **** indicates p value <0.01 between PP and 2% branched P(1-VAL-8), and between PP and 2% branched P(1-VAL-10) samples, n=7). Capsule thickness values were also assessed for 3 month samples (FIG. 15B) (* indicates p value <0.01 between P(1-VAL-8) and PP samples. ** indicates p value <0.01 between P(1-VAL-10) and PP and between P(1-VAL-10) and 2% branched P(1-VAL-10) samples. *** indicates p value <0.01 between P(1-VAL-12) and PP samples. **** indicates p value <0.01 between PP and 2% branched P(1-VAL-8), and between PP and 2% branched P(1-VAL-10) samples, n=7).

FIG. 16 is a ¹H-NMR overlay of P(1-VAL-8) and PHE8 poly(1-VAL-8) PEU polymers showing successful synthesis. The monomer molar composition in the afforded polymers was calculated from the characteristic ‘a’ peaks from L-valine and the methylene peaks from L-phenylalanine denoted ‘1’. As the molar composition rises from 10-30% more of the L-phenylalanine peaks can be observed.

FIG. 17 is a ¹H-NMR overlay of P(1-VAL-8) and PHE6 poly(1-VAL-8) PEU polymers showing successful synthesis. The monomer molar composition in the afforded polymers was calculated from the characteristic ‘a’ peaks from L-valine and the methylene peaks from L-phenylalanine denoted ‘1’. As the molar composition rises from 10-30% more of the L-phenylalanine peaks can be observed.

FIG. 18 is a ¹³C-NMR spectrum of PEU copolymers according to one or more embodiments of the present invention showing successful synthesis. The characteristic L-valine methyl peaks are observed between 18-20 ppm while the L-phenylalanine ring peaks can be seen with peaks between 125-136 ppm.

FIG. 19 is a graph showing the results of TGA performed to determine the degradation temperature (T_{d}) for copolymers according to one or more embodiments of the present invention. The T_{d} for each copolymer was high enough to allow for these materials to be thermally processed through compression molding.

FIG. 20 is a graph showing the results of SEC analysis of each PEU according to one or more embodiments of the present invention comparing the initial molecular weights. The molecular weights and molar mass distributions for six copolymers are close for step-growth polymerization with Dm values between 1.4-1.7.

FIGS. 21A-B are graphs showing the results of DSC analysis of the copolymer analogues according to one or more embodiments of the present invention showing that the glass transition temperatures for these materials are near physiological conditions. Two separate DSC instruments were used to assess the T_{g} (TA Q10 (FIG. 21A) and TA Q200 (FIG. 21B). The glass transition temperatures are significantly lower than the degradation temperature which allowed for these materials to be thermally processed through compression molding without degradation.

FIG. 22 is a graph showing the results of water uptake experiments on six copolymers according to one or more embodiments of the present invention. All six copolymers were assessed to determine how incorporation of L-phenylalanine and change in diol change length would affect water uptake. An increase in L-phenylalanine led to a drop in water uptake for the PHE6 P(1-VAL-8) polymers. The opposite trend was observed for the PHE8 P(1-VAL-8) polymers.

FIG. 23 is a stress-strain curve of copolymer PEUs according to one or more embodiments of the present invention obtained from tensile tests performed at 25° C. with a constant strain rate of 25.4 mm/min. All uniaxial mechanical data were extrapolated from the curves which are representative of 4-6 samples for each polymer.

FIG. 24 is a graph showing Young's moduli values for polymers according to one or more embodiments of the present invention extrapolated at 10% strain (* indicates p value <0.05 between PP and 20% PHE8 P(1-VAL-8), between PP and 30% PHE8 P(1-VAL-8), and between PP and 30% PHE6 P(1-VAL-8)). ** indicates p value <0.05 between P(1-VAL-8) and 20% PHE8 P(1-VAL-8), between P(1-VAL-8) and 30% PHE8 P(1-VAL-8). *** indicates p value <0.05 between 10% PHE8 P(1-VAL-8) and 20% PHE8 P(1-VAL-8), between 10% PHE8 P(1-VAL-8) and 30% PHE8 P(1-VAL-8), between 10% PHE8 P(1-VAL8) and 20% PHE6 P(1-VAL-8), and between 10% PHE8 P(1-VAL8) and 30% PHE6 P(1-VAL-8). **** indicated p value <0.05 between 20% PHE8 P(1-VAL-8) and 10% PHE6 P(1-VAL-8), between 20% PHE8 P(1-VAL-8) and 20% PHE6 P(1-VAL-8), and between 20% PHE8 P(1-VAL-8) and 30% PHE6 P(1-VAL-8). ***** indicates p value <0.05 between 30% PHE8 P(1-VAL-8) and 10% PHE6 P(1-VAL-8), between 30% PHE8 P(1-VAL-8) and 20% PHE6 P(1-VAL-8), and between 30% PHE8 P(1-VAL-8) and 30% PHE6 P(1-VAL-8), n=4-6 samples).

FIG. 25 is a graph showing the results of yield stress (σ_{y}) for polymers and copolymers according to one or more embodiments of the present invention measured at the yield point (* indicates p value <0.05 between PP and 20% PHE8 P(1-VAL-8) and between PP and 30% PHE8 P(1-VAL-8). ** indicates p value <0.05 between P(1-VAL-8) and 20% PHE8 P(1-VAL-8), between P(1-VAL-8) and 30% PHE8 P(1-VAL-8). *** indicates p value <0.05 between 10% PHE8 P(1-VAL-8) and 20% PHE8 P(1-VAL-8), between 10% PHE8 P(1-VAL-8) and 30% PHE8 P(1-VAL-8), and between 10% PHE8 P(1-VAL8) and 30% PHE6 P(1-VAL-8). **** indicated p value <0.05 between 20% PHE8 P(1-VAL-8) and 10% PHE6 P(1-VAL-8), between 20% PHE8 P(1-VAL-8) and 20% PHE6 P(1-VAL-8), and between 20% PHE8 P(1-VAL-8) and 30% PHE6 P(1-VAL-8). ***** indicates p value <0.05 between 30% PHE8 P(1-VAL-8) and 10% PHE6 P(1-VAL-8), between 30% PHE8 P(1-VAL-8) and 20% PHE6 P(1-VAL-8), and between 30% PHE8 P(1-VAL-8) and 30% PHE6 P(1-VAL-8), n=4-6 samples).

FIG. 26 is a graph showing the results of yield strain (ε_{y}) testing done on polymers and copolymers according to one or more embodiments of the present invention measured at the yield point (* indicates a p value <0.05 between PP and 20% PHE8 P(1-VAL-8), between PP and 30% PHE8 P(1-VAL-8), and between PP and 20% PHE6 P(1-VAL-8). ** indicates p value <0.05 between P(1-VAL-8) and 20% PHE8 P(1-VAL-8). *** indicates a p value <0.05 between 10% PHE8 P(1-VAL-8) and 20% PHE8 P(1-VAL-8), n=4-6 samples).

FIGS. 27A-E are images and graphs outlining ball-burst testing procedures adapted from American Society for Testing and Materials (ASTM) standards (ASTM D 3787-07 (2007)) and used herein. Burst-test clamp surface was (FIG. 27A) cleaned using tissue-paper. PEU-ECM composite films were submerged in PBS for 5 minutes prior to being placed on the bottom clamp. Films were then fastened with a top clamp followed by a screw in clamp (FIGS. 27B-C) to ensure that film slippage would not occur during testing. Burst-testing was performed (FIG. 27D) at an extension rate of 25.4 mm/min. The force versus extension data was recorded up until films burst (FIG. 27E).

FIG. 28 is a graph showing the force versus extension data for ECM and ECM/PEU composite films subjected to the ball-burst testing procedures outlined in FIGS. 27A-E above, which were adapted from ASTM D 3787-07 (2007) standards.

FIG. 29 is a graph showing force at break for ECM and PEU-ECM composite films recorded from the force versus extension curves (* indicates p value <0.05 between ECM and 10% PHE8 P(1-VAL-8) and between ECM and 30% PHE8 P(1-VAL-8). ** indicates p value <0.05 between 10% PHE6 P(1-VAL-8) and 10% PHE8 P(1-VAL-8) and between 10% PHE6 P(1-VAL-8) and 30% PHE8 P(1-VAL-8). *** indicated p value <0.05 between 10% PHE8 P(1-VAL-8) and 20% PHE8 P(1-VAL-8). **** indicates p value <0.05 between 20% PHE8 P(1-VAL-8) and 30% PHE8 P(1-VAL-8), n=3 samples). No other samples were significantly different.

FIG. 30 is a graph showing extension at break for ECM and PEU-ECM composite films according to one or more embodiments of the present invention recorded from the force versus extension curves (* indicates p value <0.05 between 10% PHE6 P(1-VAL-8) and 10% PHE8 P(1-VAL-8), n=3 samples). No other samples were significantly different.

FIG. 31 is a graph showing the results of relative stiffness analysis performed on PEU-ECM composite films recorded by dividing the force at break by the extension at break. There was no significant difference among any samples which indicates that the films force and extension at break is proportional to that of free standing ECM.

FIG. 32 is a graph showing force versus extension curves for free-standing films obtained from ball-burst testing. Samples were clamped in to a ball-burst apparatus and then extended at a constant rate of 25.4 mm/min. Curves indicate the force and extension where sample failure occurred. Curves are representative of n=3 samples.

FIG. 33 is a graph showing the relative stiffness of free-standing films recorded by dividing the force at break by the extension at break (* indicates p value <0.05 between ECM and all six copolymers. ** indicates p value <0.05 between 10% PHE6 P(1-VAL-8) and 20% PHE8 P(1-VAL-8), n=3 samples). No other samples were significantly different.

FIG. 34 is a graph showing force at break for free-standing films according to one or more embodiments of the present invention recorded from the force versus extension curves (* indicates p value <0.05 between ECM and 10% PHE8 P(1-VAL-8). ** indicates p value <0.05 between 10% PHE6 P(1-VAL-8) and 10% PHE8 P(1-VAL-8) and between 10% PHE6 P(1-VAL-8) and 20% PHE8 P(1-VAL-8), n=3 samples). No other samples were significantly different.

FIG. 35 is a graph showing the results of extension at break for free-standing films were recorded from the force versus extension curves (* indicates p value <0.05 between ECM and 10% PHE6 P(1-VAL-8), between ECM and 20% PHE6 P(1-VAL-8), between 30% PHE6 P(1-VAL-8), and between ECM and 30% PHE8 P(1-VAL-8). ** indicates p value <0.05 between 20% PHE6 P(1-VAL-8) and 10% PHE8 P(1-VAL-8) and between 20% PHE6 P(1-VAL-8) and 20% PHE8 P(1-VAL-8), n=3 samples). No other samples were significantly different.

FIG. 36 is a ¹H-NMR spectra of 1-VAL-8 monomer showing successful synthesis based on the characteristic L-valine methyl peak denoted ‘k’ and p-toluenesulfonic acid aromatic peaks. Integration confirms that this monomer is a bifunctional monomer with two protonated amine moieties.

FIG. 37 is a ¹H-NMR spectra for 1-PHE-6 monomer showing successful synthesis based on the characteristic L-phenylalanine aromatic peaks denoted ‘d, e, f’ and the p-toluenesulfonic acid aromatic peaks. Integration confirms that this monomer is a bifunctional monomer with two protonated amine moieties.

FIG. 38 is a ¹H-NMR spectra for 1-PHE-8 monomer showing successful synthesis based on the characteristic L-phenylalanine aromatic peaks denoted ‘d, e, f’ and the p-toluenesulfonic acid aromatic peaks. Integration confirms that this monomer is a bifunctional monomer with two protonated amine moieties.

### DETAILED DESCRIPTION OF THE ILLUSTRATIVE EMBODIMENTS

In one or more embodiments, the present invention is directed to an implantable polymer mesh for use in hernia and other soft tissue repair made using amino acid based poly(ester urea) polymers that have tunable degradation rates, sufficient mechanical properties throughout the healing process, and elicit a limited foreign body response. In some embodiments, the implantable polymer mesh of the present invention is made using linear or branched L-valine based PEUs and displays mechanical properties similar to poly(propylene) (PP), but with significantly less fibrous capsule formation at the wound healing site. In some other embodiments, the implantable polymer mesh of the present invention is made using linear or branched L-valine based PEUs as a composite with an extracellular matrix (ECM). In some other embodiments, the implantable polymer mesh of the present invention is made using L-valine-co-L-phenylalanine PEUs, either alone or as a composite with an extracellular matrix (ECM). In various embodiments, these amino acid-based PEU materials can be formed into implantable polymer mesh having a conventional size and shape for use in hernia and other soft tissue repair by a wide variety of techniques including conventional compression molding, vacuum molding, blade coating, flow coating, and/or solvent casting.

As set forth above, in various embodiments the implantable polymer mesh of the present invention comprises one or more amino acid based poly(ester urea) polymer. In various embodiments, these amino acid-based poly(ester urea) polymers will comprise the residue of two or more amino acid based polyester monomers separated by urea groups. As used herein, the term “residue(s)” is used to refer generally to the part of a monomer or other chemical unit that has been incorporated into a polymer or large molecule. Accordingly, the terms “polyester monomer residue,” “amino acid-based polyester monomer residue,” “amino acid-based polyester residue,” “residue of . . . [an] amino acid based polyester monomer(s),” and “monomer residue,” are used interchangeably to refer to the part of an amino acid-based polyester monomer that is incorporated into amino acid based poly(ester urea) polymers of the implantable polymer mesh of the present invention. In addition, each amino acid-based polyester monomer residue forming these amino acid based poly(ester urea) polymers may also be referred to herein as a “segment” of that polymer. As will be appreciated, these segments are connected with urea linkages to form the PEU polymer.

In one or more embodiments, these amino acid based polyester monomer residues each comprise the residues of two (if linear) or three or more (if branched) amino acid residues, each separated the others by from about 2 to about 20 carbon atoms. In one or more embodiments, these amino acid based polyester monomer residues each comprise the residue of a diol (if linear) or a polyol having three or more available OH functional groups (if branched), wherein each OH group is separated the others by from about 2 to about 20 carbon atoms. As used herein, the term amino acid refers to a chemical compound having an amine group, a carboxyl group, and a pendent R group which may be hydrogen or an organic group. In various embodiments, the R group may comprise an alkyl, aryl, thiol, sulfide, hydroxyl, hydrogen, selenol, imidazole, or indole, group. In one or more embodiments, these amino acids may be any naturally occurring α-amino acid, but the invention is not so limited and, in some embodiments, non-naturally occurring amino acids having non-toxic and resorbable degradation products may be used. As will be apparent, the terms “residue of an amino acid” and “amino acid residue,” as well as references to the “residue” of a particular amino acid (e.g., “valine residue” or “residue of valine”), are used interchangeably herein to refer to the part of an amino acid that is incorporated into the structure of the amino acid based polyester monomers monomers and will ordinarily include the NH of the amino group, the carboxyl group, and side chain of the amino acid. Similarly, the terms “residue of a diol” and “diol residue,” as well as reference to the “residue” of a particular diol, are used interchangeably to refer to the part of the diol used to form the amino acid-based polyester monomers that is incorporated into that monomer's structure and the terms “residue of a polyol” and “polyol residue,” as well as references to the “residue” of a particular polyol, are likewise used interchangeably to refer to the part of the polyol used to form the amino acid-based polyester monomers that are incorporated into that monomer's structure.

In one or more embodiment, the amino acid based polyester monomer residues used to form the amino acid based polyester polymers of the implantable polymer mesh of the present invention may comprise the residue of L-valine, L-leucine, L-isoleucine, L-serine, L-alanine, L-glycine, L-aspartic acid, L-asparagine, L-arginine, L-phenylalanine, L-methionine, or benzyl protected L-tyrosine, separated by from about 2 to about 20 carbon atoms, as set forth above. In some of these embodiments, the amino acid residues will be separated by from about 2 to about 18 carbon atoms, in other embodiments from about 2 to about 16 carbon atoms, in other embodiments from about 2 to about 14 carbon atoms, in other embodiments from about 2 to about 10 carbon atoms, in other embodiments from about 2 to about 8 carbon atoms, in other embodiments from about 4 to about 20 carbon atoms, in other embodiments from about 6 to about 20 carbon atoms, in other embodiments from about 8 to about 20 carbon atoms, in other embodiments from about 10 to about 20 carbon atoms.

In some of these embodiments, the amino acid based polyester monomer residues will each comprise the residues of two (if linear) or three or more (if branched) valine residues, each separated by from about 2 to about 20 carbon atoms. In some embodiments, the amino acid based polyester monomer residues each comprise the residues of two (if linear) or three or more (if branched) valine residues, each separated by from about 2 to about 14 carbon atoms. In some embodiments, the amino acid based polyester monomer residues each comprise the residues of two phenylalanine residues, each separated by from about 2 to about 20 carbon atoms. In some embodiments, the amino acid based polyester monomer residues each comprise the residues of two phenylalanine residues, each separated by from about 2 to about 14 carbon atoms.

In one or more of these embodiments, the amino acid based polyester polymers used to form the implantable polymer mesh of the present invention will comprise a homopolymer of a valine based amino acid monomers and will have the formula:

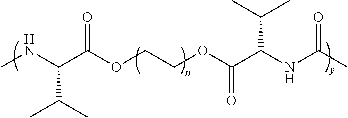

where n is an integer from 1 and 12; and y is an integer from about 10 to about 1000. In some of these embodiments, n may be an integer from about 1 to about 10, in other embodiments, from about 1 to about 8, in other embodiments, from about 1 to about 6, in other embodiments, from about 1 to about 4, in other embodiments, from about 2 to about 12, in other embodiments, from about 4 to about 12, and in other embodiments, from about 6 to about 12. In some of these embodiments, y may be an integer from about 10 to about 750, in other embodiments, from about 10 to about 500, in other embodiments, from about 10 to about 400, in other embodiments, from about 10 to about 300, in other embodiments, from about 10 to about 200, in other embodiments, from about 10 to about 100, in other embodiments, from about 10 to about 50, in other embodiments, from about 100 to about 750, in other embodiments, from about 200 to about 750, in other embodiments, from about 300 to about 750, in other embodiments, from about 400 to about 750, in other embodiments, from about 500 to about 750.

In a variety of embodiments, the amino acid-based poly(ester urea) used to form the implantable polymer mesh of various embodiments of the present invention may be a copolymer comprising two or more different types of monomer segments, each type containing the residue of a different types of amino acid based polyester monomer. As used herein, reference to a particular “type” of amino acid-based polyester or monomer segment is intended to refer to one or more amino acid-based polyester segments formed from and containing the residue of the same amino acid-based polyester monomer, with each segment in a particular type having the identical structure and function. In some embodiments, the amino acid-based poly(ester urea) used to form the implantable polymer mesh of various embodiments of the present invention will comprise two different types of amino acid-based polyester monomers segments. In some other embodiments, the amino acid-based poly(ester urea) used to form the implantable polymer mesh of various embodiments of the present invention will comprise three or more different types of amino acid-based polyester monomers segments.

In some of these embodiments, the amino acid-based poly(ester urea) will comprise a plurality of a first type of polyester segments all comprising a first type of amino acid based polyester residue, which may be linear or branched, and a plurality of a second type of polyester segments all comprising a second type of amino acid based polyester residue, which may be linear or branched, separated by urea groups. As set forth above, these first and second types of polyester segments are all connected by urea linkages.

In some of these embodiments, the amino acid based polyester residues in the first type of polyester segments may comprise the residue of a linear amino acid based diester monomer comprising the residues of two amino acids separated by from about 2 to about 20 carbon atoms, as described above. In one or more these embodiments, each of the amino acids in said first type of amino acid based polyester monomer residues may be residues of L-valine, L-leucine, L-isoleucine, L-serine, L-alanine, L-glycine, L-aspartic acid, L-asparagine, L-arginine, L-phenylalanine, L-methionine, benzyl protected L-tyrosine or a combination thereof, as set forth above. In some of these embodiments, the two amino acid residues in the first type of amino acid based polyester monomer residues are the same amino acid. In some other embodiments, the two amino acid residues in the first type of amino acid based polyester monomer residues are different amino acids. In one or more embodiments, the amino acid based polyester residues in the first type of polyester segments will comprise two valine residues separated by from about 2 to about 20 carbon atoms, as described above. In one or more embodiments, the amino acid based polyester residues in the first type of polyester segments will comprise two valine residues separated by from about 2 to about 14 carbon atoms, as described above.

In various embodiments, the amino acid based polyester residues in the first type of polyester segments may comprise, without limitation, the residue of di-p-toluenesulfonic acid salts of bis(L-phenylalanine)-ethane 1,2-diester, di-p-toluenesulfonic acid salts of bis(L-phenylalanine)-butane 1,4-diester, di-p-toluenesulfonic acid salts of bis(L-phenylalanine)-hexane 1,6-diester, di-p-toluenesulfonic acid salts of bis(L-phenylalanine)-octane 1,8-diester, di-p-toluenesulfonic acid salts of bis(L-phenylalanine)-decane 1,10-diester, di-p-toluenesulfonic acid salts of bis(L-phenylalanine)-dodecane 1,12-diester, and di-p-toluenesulfonic acid salts of bis(L-phenylalanine)-tetradecane 1,14-diester, di-p-toluenesulfonic acid salts of bis(L-valine)-ethane 1,2-diester, di-p-toluenesulfonic acid salts of bis(L-valine)-butane 1,4-diester, di-p-toluenesulfonic acid salts of bis(L-valine)-hexane 1,6-diester, di-p-toluenesulfonic acid salts of bis(L-valine)-octane 1,8-diester, di-p-toluenesulfonic acid salts of bis(L-valine)-decane 1,10-diester, di-p-toluenesulfonic acid salts of bis(L-valine)-dodecane 1,12-diester, or di-p-toluenesulfonic acid salts of bis(L-valine)-tetradecane 1,14-diester monomer.

In some other embodiments, amino acid based polyester residues in the first type of polyester segments may be the residue of a branched polyester monomer that has three or more amino-acid residues as defined above, each separated from the other amino acid residues by from about 2 to about 20 carbon atoms, as described above. In various embodiments, the first type of amino acid based polyester monomer residues in the first type of polyester segments will contain the residues of three or more of the same amino acid. In some other embodiments, the first type of amino acid based polyester monomer residues will contain the residues of three or more amino acids that are not all the same amino acid. In one or more of these embodiments, the first type of amino acid based polyester residues may be the residues of a branched polyester monomer that has three or more valine residues, each separated from the others by from about 2 to about 20 carbon atoms as described above. In some of these embodiments, the three or more amino acids residues in the first type of polyester segment may be separated from each other by the residue of a branched compound including, but not limited to, 2-butene-1,4-diol, 3,4-dihydroxy-1-butene, 7-octene-1,2-diol, 3-hexene-1,6-diol, 1,4-butynediol, trimethylolpropane allyl ether, 3-allyloxy-1,2-propanediol, 2,4-hexadiyne-1,6-diol, 2-hydroxymethyl-1,3-propanediol, 1,1,1-tris(hydroxymethyl)propane, 1,1,1-tris(hydroxymethyl)ethane, pentaerythritol, di(trimethylolpropane) dipentaerythritol and combinations thereof. In some of these embodiments, the residue of the branched compound comprises the residue of a polyol having three or more reactive hydroxyl groups capable of bonding to an amino acid. In various embodiments, the amino acid based polyester residues in the first type of polyester segments may include, without limitation, the residue of tri-o-benzyl-L-tyrosine-1,1,1-trimethylethane.

As set forth above, in these embodiments, the amino acid-based poly(ester urea) used to form the implantable polymer mesh of the present invention will also comprise a plurality of a second type of polyester segments will all contain the same type of amino acid based polyester monomer residue, which will be different from the type of amino acid based polyester monomer residues comprising the first type of polyester segments, as described above. In various embodiments, the second type of amino acid based polyester monomer residue may be linear or branched. In some of these embodiments, the amino acid based polyester monomer residues forming the second type of polyester segment may comprise the residue of a linear diester monomer comprising the residues of two amino acids separated by from each other by from about 2 to about 20 carbon atoms, as described above.

In some embodiments, all of the amino acid residues in the type of amino acid based polyester monomer residue forming the second type of polyester segment are the same amino acid, but this need not be the case. In one or more embodiments, each of the amino acid residues in the type of amino acid based polyester monomer residue forming the second type of polyester segments may be L-valine, L-leucine, L-isoleucine, L-serine, L-alanine, L-glycine, L-aspartic acid, L-asparagine, L-arginine, L-phenylalanine, L-methionine, benzyl protected L-tyrosine, or a combination thereof. In one or more embodiments, the type of amino acid based polyester monomer residues forming the second type of polyester segment will comprise two valine residues separated by from about 2 to about 20 carbon atoms, as described above. In one or more embodiments, the type of amino acid based polyester monomer residues forming the second type of polyester segment will comprise three valine residues, each separated from the others by from about 2 to about 20 carbon atoms. In one or more embodiments, the second type of amino acid based polyester monomer residues will comprise two phenylalanine residues separated by from about 2 to about 14 carbon atoms, as described above. In some of these embodiments, the three or more amino acids residues in the first type of polyester segment may be separated from each other by the residue of a branched compound including, but not limited to, 2-butene-1,4-diol, 3,4-dihydroxy-1-butene, 7-octene-1,2-diol, 3-hexene-1,6-diol, 1,4-butynediol, trimethylolpropane allyl ether, 3-allyloxy-1,2-propanediol, 2,4-hexadiyne-1,6-diol, 2-hydroxymethyl-1,3-propanediol, 1,1,1-tri s(hydroxymethyl)propane, 1,1,1-tris(hydroxymethyl)ethane, pentaerythritol, di(trimethylolpropane) dipentaerythritol and combinations thereof. In some of these embodiments, the residue of the branched compound comprises the residue of a polyol having three or more reactive hydroxyl groups capable of bonding to an amino acid. In various embodiments, the amino acid based polyester residues in the first type of polyester segments may include, without limitation, the residue of tri-o-benzyl-L-tyrosine-1,1,1-trimethylethane.

In various embodiments, the type of amino acid based polyester monomer residue forming the second type of polyester segment may include, without limitation, the residue of: di-p-toluenesulfonic acid salts of bis(L-phenylalanine)-ethane 1,2-diester, di-p-toluenesulfonic acid salts of bis(L-phenylalanine)-butane 1,4-diester, di-p-toluenesulfonic acid salts of bis(L-phenylalanine)-hexane 1,6-diester, di-p-toluenesulfonic acid salts of bis(L-phenylalanine)-octane 1,8-diester, di-p-toluenesulfonic acid salts of bis(L-phenylalanine)-decane 1,10-diester, di-p-toluenesulfonic acid salts of bis(L-phenylalanine)-dodecane 1,12-diester, and di-p-toluenesulfonic acid salts of bis(L-phenylalanine)-tetradecane 1,14-diester, di-p-toluenesulfonic acid salts of bis(L-valine)-Ethane 1,2-diester, di-p-toluenesulfonic acid salts of bis(L-valine)-butane 1,4-diester, di-p-toluenesulfonic acid salts of bis(L-valine)-hexane 1,6-diester, di-p-toluenesulfonic acid salts of bis(L-valine)-octane 1,8-diester, di-p-toluenesulfonic acid salts of bis(L-valine)-decane 1,10-diester, di-p-toluenesulfonic acid salts of bis(L-valine)-dodecane 1,12-diester, or di-p-toluenesulfonic acid salts of bis(L-valine)-tetradecane 1,14-diester monomer.

While the implantable polymer mesh of the present invention in places as comprising two types of amino acid based polyester monomer segments, it should be understood that the invention is not so limited, and implantable polymer mesh comprising three or more different types amino acid based polyester monomer segments are possible and within the scope of the present invention.

In one or more embodiment, the amino acid-based poly(ester urea) used to form the implantable polymer mesh of various embodiments of the present invention may have the formula:

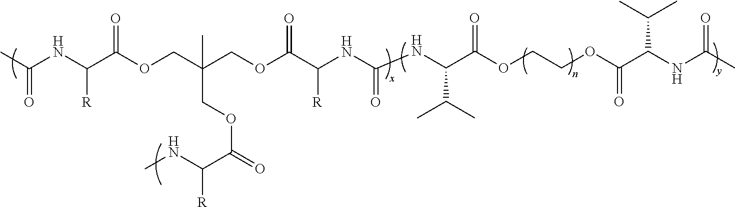

where n is an integer from 1 and 12; each R is the side chain of any amino acid except proline; x is a mole fraction of from about 0.001 to about 0.01; and y is a mole fraction from about 0.90 to about 0.999. In one or more embodiments, R may be is —CH₃, —(CH₂)₃NHC(NH₂)C═NH, —CH₂CONH₂, —CH₂COOH, —CH₂SH, —(CH₂)₂COOH, —(CH₂)₂CONH₂, —NH, —CH₂C═CH—N═CH—NH, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C═CH—NH-Ph, —CH₂-Ph-OH, or —CH(CH₃)₂. In some of these embodiments, n may be an integer from about 1 to about 10, in other embodiments, from about 1 to about 8, in other embodiments, from about 1 to about 6, in other embodiments, from about 1 to about 4, in other embodiments, from about 2 to about 12, in other embodiments, from about 4 to about 12, and in other embodiments, from about 6 to about 12.

In these embodiments, it is strongly preferred that the branched segments (x in the above formula) not exceed 10 mole percent of the PEU polymer. In some of these embodiments, x may be a mole fraction from about 0.001 to about 0.01, in other embodiments, from about 0.001 to about 0.09, in other embodiments, from about 0.001 to about 0.08, in other embodiments, from about 0.001 to about 0.06, in other embodiments, from about 0.001 to about 0.04, in other embodiments, from about 0.001 to about 0.02, in other embodiments, from about 0.001 to about 0.01, in other embodiments, from about 0.01 to about 0.09, in other embodiments, from about 0.03 to about 0.09, in other embodiments, from about 0.05 to about 0.09, and in other embodiments, from about 0.06 to about 0.09. In some of these embodiments, y may a mole fraction from about 0.92 to about 0.999, in other embodiments, from about 0.94 to about 0.999, in other embodiments, from about 0.96 to about 0.999, in other embodiments, from about 0.98 to about 0.999, in other embodiments, from about 0.90 to about 0.98, in other embodiments, from about 0.90 to about 0.96, and in other embodiments, from about 0.90 to about 0.94.

In one or more embodiment, the amino acid-based poly(ester urea) used to form the implantable polymer mesh of various embodiments of the present invention may have the formula:

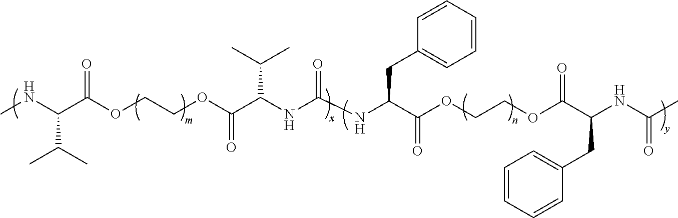

where n and m can each be an integer from about 1 to about 12; x is a mole fraction of from about 0.05 to about 0.95; and y is a mole fraction from about 0.05 to about 0.95. In some of these embodiments, each individual m and n may be an integer from about 1 to about 10, in other embodiments, from about 1 to about 8, in other embodiments, from about 1 to about 6, in other embodiments, from about 1 to about 4, in other embodiments, from about 2 to about 12, in other embodiments, from about 4 to about 12, and in other embodiments, from about 6 to about 12.

In some of these embodiments, x may be a mole fraction from about 0.05 to about 0.85, in other embodiments, from about 0.05 to about 0.75, in other embodiments, from about 0.05 to about 0.65, in other embodiments, from about 0.05 to about 0.55, in other embodiments, from about 0.05 to about 0.45, in other embodiments, from about 0.05 to about 0.35, in other embodiments, from about 0.15 to about 0.95, in other embodiments, from about 0.25 to about 0.95, in other embodiments, from about 0.35 to about 0.95, in other embodiments, from about 0.45 to about 0.95, and in other embodiments, from about 0.55 to about 0.95. In some of these embodiments, y may be a mole fraction from about 0.05 to about 0.85, in other embodiments, from about 0.05 to about 0.75, in other embodiments, from about 0.05 to about 0.65, in other embodiments, from about 0.05 to about 0.55, in other embodiments, from about 0.05 to about 0.45, in other embodiments, from about 0.05 to about 0.35, in other embodiments, from about 0.15 to about 0.95, in other embodiments, from about 0.25 to about 0.95, in other embodiments, from about 0.35 to about 0.95, in other embodiments, from about 0.45 to about 0.95, and in other embodiments, from about 0.55 to about 0.95. In one or more embodiments, y is a mole fraction from about 0.05 to about 0.30.

The methods for preparing the amino acid based polyester monomers and forming the amino acid based poly(ester urea)s described above are not particularly limited. In various embodiments, the amino acid based polyester monomers may be formed by reacting the selected amino acids with a suitable diol or other polyol. In some embodiments, the acid salt of these amino acid based polyester monomers used is to form the amino acid based poly(ester urea)s described above, may be synthesized as shown in Scheme 1, below.

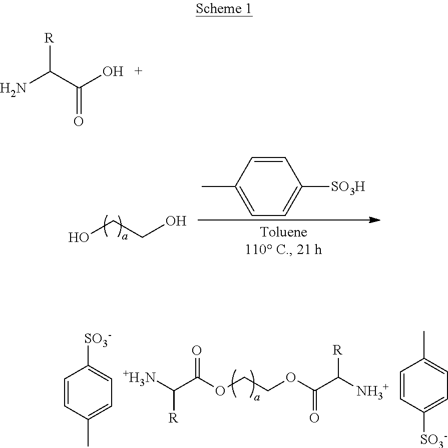

where a is an integer from 1 to 12 and R is the amino acid side chain of an amino acid as described above. In various embodiments, each R may be —CH₃, —(CH₂)₃NHC(NH₂)C═NH, —CH₂CONH₂, —CH₂COOH, —CH₂SH, —(CH₂)₂COOH, —(CH₂)₂CONH₂, —I, —CH₂C—CH—N—=CH—NH, —CH(CH₃)CH₂CH₃, —CH₂CH(CH₃)₂, —(CH₂)₄NH, —(CH₂)₂SCH₃, —CH₂Ph, —CH₂OH, —CH(OH)CH₃, —CH₂—C=CH—NH-Ph, —CH₂—Pb—OH, —CH(CH₃)₂, —CH₂C₆H₄OCH₂C₆H₅, or —CH₂PhI. In some embodiments, the amino acid based polyester monomers described above may be synthesized as described in U.S. Published Patent Applications Numbers 2016/0250382, 2017/0081476, 2017/0210852, published International Patent Application No. WO 2017/189534 and/or U.S. Pat. Nos. 9,745,414 and 9,988,492, the disclosures of which are incorporated herein by reference in their entirety.

As will be apparent to those of skill in the art, the toluene sulphonic acid (pTSA) in the reaction of Scheme 1 is necessary to protonate the amine on the amino acid to ensure that transamidation reactions do not occur at higher conversions. As used herein, the terms “counter-ion protected amino acid based polyester monomer,” and “acid salt of [the/an] amino acid based polyester monomer,” are used interchangeably to refer to an amino acid based polyester monomer as described above wherein the amine groups on the amino acid-based polyester monomers are protected by one or more protecting anions. As follows, the terms “counter-ion protected amino acid based diester monomer,” “counter-ion protected linear amino acid based polyester monomer,” “linear counter-ion protected amino acid based polyester monomer,” “acid salt of [the/an] amino acid based diester monomer,” and “acid salt of [the/an] linear amino acid based polyester monomer,” are used interchangeably to refer to a linear amino acid based diester monomer as described above wherein the amine groups on the amino acid-based polyester monomers are protected by one or more protecting anions and the terms “counter-ion protected branched amino acid based polyester monomer,” “branched counter-ion protected amino acid based polyester monomer,” and “acid salt of [the/an] branched amino acid based polyester monomer,” are used interchangeably to refer to a branched amino acid based polyester monomer as described above wherein the amine groups on the amino acid-based polyester monomers are protected by one or more protecting anions.

Accordingly, a suitable acid or other source of counter-ions may be added to the solution prior to or during formation of the polyester monomer. One of ordinary skill in the art will be able to select a suitable counter-ion without undue experimentation. Materials capable of producing suitable protecting counter-ions may include without limitation, p-toluene sulfonic acid monohydrate, chlorides, bromides, acetates. trifloroacetate, or combinations thereof. In some embodiments, the acid used may be p-toluene sulfonic acid monohydrate. In some embodiments, the acid used may be HCl.

In some other embodiments, a linear polyester monomer having a functionalizable side chain formed by reacting a branched diol containing a functionalizable side chain with two suitable amino acids. Suitable branched diol starting materials containing a functionalizable side chain may include, without limitation, 2-butene-1,4-diol, 3,4-dihydroxy-1-butene, 7-octene-1,2-diol, 3-hexene-1,6-diol, 1,4-butynediol, trimethylolpropane allyl ether, 3-allyloxy-1,2-propanediol, 2,4-hexadiyne-1,6-diol, and combinations thereof.

In some other embodiments, the acid salt of the branched amino acid based polyester monomers used to form the amino acid based poly(ester urea)s described above may be synthesized as shown in Scheme 2, below.

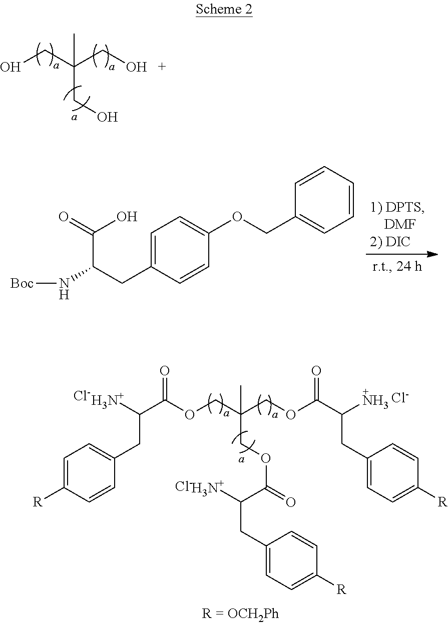

where a is an integer from 1 to 20. In one or more embodiments, the acid salt of the branched amino acid based polyester monomers used to form the amino acid based poly(ester urea)s described above may be synthesized as set forth in Yu, J.; Lin, F.; Becker, M. L., “Branched amino acid based poly(ester urea)s with tunable thermal and water uptake properties.” Macromolecules 2015 DOI: 10.1021/acs.macromol.5b00376 and/or U.S. Pat. No. 9,745,414, the disclosures of which is incorporated herein by reference. In some embodiments, the branched amino acid based polyester monomers may be synthesized as shown in Example 6.

In one or more embodiment, the branched polyester monomer was formed through the esterification between a polyol having three or more available hydroxyl groups, and three or more suitable amino acids, as shown in Scheme 2, above. Suitable branched polyols may include, without limitation, 1,1,1-tris(hydroxymethyl)propane, 1,1,1-tris(hydroxymethyl)ethane, dipentaerythritol, pentaerythritol, 2-hydroxymethyl-1,3-propanediol, di(trimethylolpropane), and combinations thereof.

Suitable amino acids may include, without limitation, L-valine, L-leucine, L-isoleucine, L-serine, L-alanine, L-glycine, L-aspartic acid, L-asparagine, L-arginine, L-phenylalanine, L-methionine, benzyl protected L-tyrosine or a combination thereof. As will be appreciated by those of skill in the art, these amino acids may be protected in any conventional way. In the amino acid shown in Scheme 2 above, for example, the amine is protected with a tert-butyloxycarbonyl (Boc) protecting group and the ether linkage on the side chain of the tyrosine molecule is protected with a benzyl group.

In these embodiments, the polyol, the amino acid, and a suitable base catalyst, such as 4-(N,N-dimethylamino)puridinium 4-toluenesulfonate (DPTS), are dissolved in a suitable organic solvent, such as N,N-dimethylformamide (DMF), Chloroform, tetrahydrofuran (THF) or 4-methyl THF and placed in an ice bath, water bath, or other heat sink. A coupling agent, such as 1,3-diisopropyl carbodiimide (DIC), is then added and the reaction vessel allowed to gradually come to ambient temperature while stirring for 24 h to produce a crude branched boc protected amino acid based polyester monomer. The crude branched boc protected amino acid based polyester monomer is then purified and dissolved in an acid solution, such as a mixture of HCl and dioxane, to produce the acid salt of the branched amino acid based polyester monomers used to form amino acid based poly(ester urea)s used in some embodiments of the present invention. In one or more embodiments, the counter-ion protected branched amino acid-based polyester monomers may be formed as shown in U.S. Pat. No. 9,745,414, the disclosure of which in incorporated herein by reference in its entirety.

As with the linear polyester (diester) monomers described above, steps should also be taken to prevent transamidation of the ester bonds when forming the counter-ion protected branched amino acid-based polyester monomers. In some embodiments, transamidation may be prevented or limited by protecting the amine groups on the amino acid-based branched polyester monomers being formed with one or more counter-ions. Accordingly, a suitable acid or other source of counter-ions may be added to the solution prior to or during formation of the branched polyester monomer. As set forth above, one of ordinary skill in the art will be able to select a suitable counter-ion without undue experimentation. Materials capable of producing suitable protecting counter-ions may include without limitation, p-toluene sulfonic acid monohydrate, chlorides, bromides, acetates. trifloroacetate, or combinations thereof. In some embodiments, the acid used may be p-toluene sulfonic acid monohydrate. In some embodiments, the acid used may be HCl.

In various embodiments, the amino acid based poly(ester urea) polymers described above can be synthesized by the interfacial polymerization of the acid salts of the amino acid based polyester monomers described above and a urea bond forming agent. As used herein, the term interfacial polymerization refers to polymerization that takes place at or near the interfacial boundary of two immiscible fluids. The terms “urea bond forming agent” or “PEU forming compound” are used herein interchangeably to refer to a compound or other agent capable of placing a carboxyl group between two amine groups, thereby forming a urea bond and includes, without limitation, triphosgene, diphosgene, or phosgene.

In some embodiments, the amino acid based poly(ester urea) polymers described above described above may be synthesized first preparing the acid salts of one or more amino acid based polyester monomers selected to form the amino acid based poly(ester urea) polymer, as shown, for example in Schemes 1 and 2, above. As set forth above, transamidation of the ester bonds on the amino acid based polyester monomer may be prevented or limited by protecting the amine groups on the amino acid-based polyester monomers being formed using one or more counter-ions. Suitable monomers for forming the amino acid based poly(ester urea) polymers described above may include, without limitation, di-p-toluenesulfonic acid salts of bis(L-phenylalanine)-ethane 1,2-diester, di-p-toluenesulfonic acid salts of bis(L-phenylalanine)-butane 1,4-diester, di-p-toluenesulfonic acid salts of bis(L-phenylalanine)-hexane 1,6-diester, di-p-toluenesulfonic acid salts of bis(L-phenylalanine)-octane 1,8-diester, di-p-toluenesulfonic acid salts of bis(L-phenylalanine)-decane 1,10-diester, di-p-toluenesulfonic acid salts of bis(L-phenylalanine)-dodecane 1,12-diester, and di-p-toluenesulfonic acid salts of bis(L-phenylalanine)-tetradecane 1,14-diester, di-p-toluenesulfonic acid salts of bis(L-valine)-Ethane 1,2-diester, di-p-toluenesulfonic acid salts of bis(L-valine)-butane 1,4-diester, di-p-toluenesulfonic acid salts of bis(L-valine)-hexane 1,6-diester, di-p-toluenesulfonic acid salts of bis(L-valine)-octane 1,8-diester, di-p-toluenesulfonic acid salts of bis(L-valine)-decane 1,10-diester, di-p-toluenesulfonic acid salts of bis(L-valine)-dodecane 1,12-diester, di-p-toluenesulfonic acid salts of bis(L-valine)-tetradecane 1,14-diester monomer or tri-O-benzyl-L-tyrosine-1,1,1-trimethylethane.

Next, the acid salts of one or more amino acid based polyester monomers selected and a deprotecting base, preferably sodium carbonate, are dissolved in a suitable solvent. Finally a solution containing a urea bond forming compound is prepared and added to the monomer solution to form the amino acid-based poly(ester urea) polymer by interfacial polymerization. In some embodiments, the amino acid based poly(ester urea) polymers described above may be synthesized as described in U.S. Published Patent Applications Numbers 2016/0250382, 2017/0081476, 2017/0210852, published International Patent Application No. WO 2017/189534 and/or U.S. Pat. Nos. 9,745,414 and 9,988,492, the disclosure of which are incorporated herein by reference in their entirety. In one or more embodiment, the amino acid based poly(ester urea) polymers described above may be synthesized as shown in Schemes 3 and 4, below.

In one or more embodiments, the amino acid-based poly(ester urea) used to form the implantable polymer mesh of various embodiments of the present invention may have a number average molecular weight (Mₙ) of from about 10,000 g/mol to about 500,000 g/mol, as measured by size exclusion chromatography (SEC). In some embodiments, the amino acid-based poly(ester urea) used to form the implantable polymer mesh of the present invention may have a number average molecular weight (Mₙ) of from about 10,000 g/mol to about 400,000 g/mol, in other embodiments from about 10,000 g/mol to about 300,000 g/mol, in other embodiments from about 10,000 g/mol to about 200 g/mol, in other embodiments from about 10,000 g/mol to about 100,000 g/mol, in other embodiments from about 50,000 g/mol to about 500,000 g/mol, in other embodiments from about 100,000 g/mol to about 500,000 g/mol, in other embodiments from about 200,000 g/mol to about 500,000 g/mol, in other embodiments from about 300,000 g/mol to about 500,000 g/mol, and in other embodiments from about 400,000 g/mol to about 500,000 g/mol, as measured by size exclusion chromatography (SEC). Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have a weight average molecular weight (M_{w}) of from about 10,000 g/mol to about 250,000 g/mol, as measured by size exclusion chromatography (SEC). In some embodiments, the amino acid-based poly(ester urea) used to form the implantable polymer mesh of the present invention may have a weight average molecular weight (M_{w}) of from about 10,000 g/mol to about 200,000 g/mol, in other embodiments from about 10,000 g/mol to about 150,000 g/mol, in other embodiments from about 10,000 g/mol to about 125,000 g/mol, in other embodiments from about 10,000 g/mol to about 100,000 g/mol, in other embodiments from about 10,000 g/mol to about 75,000 g/mol, in other embodiments from about 25,000 g/mol to about 250,000 g/mol, in other embodiments from about 75,000 g/mol to about 250,000 g/mol, in other embodiments from about 100,000 g/mol to about 250,000 g/mol, and in other embodiments from about 125,000 g/mol to about 250,000 g/mol, as measured by size exclusion chromatography (SEC). Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have a molecular mass distribution of (Ðₘ) of from about 1.1 to about 3.0 as measured by size exclusion chromatography (SEC). In some embodiments, the amino acid-based poly(ester urea) used to form the implantable polymer mesh of the present invention may have a molecular mass distribution of (Ðₘ) of from about 1.4 to about 3.0, in other embodiments, from about 1.8 to about 3.0, in other embodiments, from about 2.2 to about 3.0, in other embodiments, from about 2.5 to about 3.0, in other embodiments, from about 1.1 to about 2.5, in other embodiments, from about 1.1 to about 2.0, and in other embodiments, from about 1.1 to about 1.5, as measured by size exclusion chromatography (SEC). Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have a glass transition temperature (T_{g}) of from about 28° C. to about 60° C. as measured by differential scanning calorimetry. In some embodiments, the amino acid-based poly(ester urea) used to form the implantable polymer mesh of the present invention may have a glass transition temperature (T_{g}) of from about 28° C. to about 50° C., in other embodiments from about 28° C. to about 45° C., in other embodiments from about 28° C. to about 40° C., in other embodiments from about 28° C. to about 35° C., in other embodiments from about 35° C. to about 57° C., in other embodiments from about 40° C. to about 57° C., and in other embodiments from about 45° C. to about 57° C., as measured by differential scanning calorimetry. Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have a degradation temperature (T_{d}) of from about 200° C. to about 350° C. as measured by thermogravimetric analysis. In some embodiments, the amino acid-based poly(ester urea) used to form the implantable polymer mesh of the present invention may have a degradation temperature (T_{d}) of from about 200° C. to about 325° C., in other embodiments from about 200° C. to about 300° C., in other embodiments from about 200° C. to about 275° C., in other embodiments from about 225° C. to about 350° C., in other embodiments from about 250° C. to about 350° C., in other embodiments from about 275° C. to about 350° C., and in other embodiments from about 300° C. to about 350° C., as measured by thermogravimetric analysis. Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have a Young's modulus of from about 10 MPa to about 500 MPa as measured by uniaxial tensile testing. In some embodiments, the amino acid-based poly(ester urea) used to form the implantable polymer mesh of the present invention may have a Young's modulus of from about 10 MPa to about 500 MPa, in other embodiments, from about 10 MPa to about 400 MPa, in other embodiments, from about 10 MPa to about 300 MPa, in other embodiments, from about 10 MPa to about 200 MPa, in other embodiments, from about 10 MPa to about 100 MPa, in other embodiments, from about 100 MPa to about 500 MPa, in other embodiments, from about 200 MPa to about 500 MPa, and in other embodiments, from about 300 MPa to about 500 MPa, as measured by uniaxial tensile testing. Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have a yield stress (σ_{y}) of from about 2 MPa to about 100 MPa as measured by uniaxial tensile testing. In some embodiments, the amino acid-based poly(ester urea) used to form the implantable polymer mesh of the present invention may have a yield stress (σ_{y}) of from about 2 MPa to about 80 MPa, in other embodiments, from about 2 MPa to about 60 MPa, in other embodiments, from about 2 MPa to about 40 MPa, in other embodiments, from about 2 MPa to about 20 MPa, in other embodiments, from about 10 MPa to about 100 MPa, in other embodiments, from about 20 MPa to about 100 MPa, in other embodiments, from about 30 MPa to about 100 MPa, and in other embodiments, from about 40 MPa to about 100 MPa, as measured by uniaxial tensile testing. Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have a yield strain (ε_{y}) of from about 1% to about 50% as measured by uniaxial tensile testing. In some embodiments, the amino acid-based poly(ester urea) used to form the implantable polymer mesh of the present invention may have a yield strain (ε_{y}) of from about 1% to about 50%, in other embodiments, from 10% to 50%, in other embodiments, from 20% to 50%, in other embodiments, from 30% to 50%, in other embodiments, from 1% to 40%, in other embodiments, from 1% to 30%, and in other embodiments from 1% to 20%, as measured by uniaxial tensile testing. Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have a force at break from about 50 N to about 500 N as measured by burst testing. In some embodiments, the amino acid-based poly(ester urea) used to form the implantable polymer mesh of the present invention may have a force at break from about 50 N to about 400 N, in other embodiments, from 50 N to 300 N, in other embodiments, from 50 N to 200 N, in other embodiments, from 100 N to 500 N, in other embodiments, from 200 N to 500 N, and in other embodiments, from 300 N to 500 N, as measured by burst testing. Here, as well as elsewhere in the specification and claims, individual range values can be combined to form additional non-disclosed ranges.

In one or more embodiments, the amino acid-based poly(ester urea) used to form the implantable polymer mesh of various embodiments of the present invention may have an extension at break from about 0.5 cm to about 5 cm as measured by burst testing. In some embodiments, the amino acid-based poly(ester urea) used to form the implantable polymer mesh of the present invention may have an extension at break from about 0.5 cm to about 5 cm, in other embodiments, from 1 cm to 5 cm, in other embodiments, from 2 cm to 5 cm, in other embodiments, from 3 cm to 5 cm, in other embodiments, from 0.5 cm to 4 cm, in other embodiments, from 0.5 cm to 3 cm, and in other embodiments, from 0.5 cm to 2 cm, as measured by burst testing. Here, as well as elsewhere in the specification and claims, individual range values can be combined to form additional non-disclosed ranges.

In one or more embodiments, the amino acid-based poly(ester urea) used to form the implantable polymer mesh of the present invention may have a relative stiffness from about 25 N/cm to about 200 N/cm, as measured by burst testing. In some embodiments, the amino acid-based poly(ester urea) used to form the implantable polymer mesh of the present invention may have a relative stiffness from about 25 N/cm to about 150 N/cm, in other embodiments, from 25 N/cm to 100 N/cm, in other embodiments, from 25 N/cm to 50 N/cm, in other embodiments, from 50 N/cm to 200 N/cm, in other embodiments, from 75 N/cm to 200 N/cm, in other embodiments, from 100 N/cm to 200 N/cm, and in other embodiments, from 125 N/cm to 200 N/cm, as measured by burst testing. Here, as well as elsewhere in the specification and claims, individual range values can be combined to form additional non-disclosed ranges.

In one or more embodiments, the amino acid-based poly(ester urea) used to form the implantable polymer mesh of the present invention may have a water uptake from about 0 mass % to about 45 mass % as measured by phosphine buffered saline solution (PBS) swelling/water uptake testing. In some embodiments, the amino acid-based poly(ester urea) used to form the implantable polymer mesh of the present invention may have a water uptake from about 0 mass % to about 45 mass %, in other embodiments, from about 5 mass % to about 45 mass %, from about 10 mass % to about 45 mass %, from about 20 mass % to about 45 mass %, from about 30 mass % to about 45 mass %, from about 0 mass % to about 35 mass %, and from about 0 mass % to about 25 mass %, as measured by phosphine buffered saline solution (PBS) swelling/water uptake testing. Here, as well as elsewhere in the specification and claims, individual range values can be combined to form additional non-disclosed ranges.

As set forth above, in some embodiments of the present invention the implantable polymer mesh may be made using linear and branched L-valine based PEUs, which display mechanical properties similar to poly(propylene) (PP), but with significantly less fibrous capsule formation at the wound healing site. For some hernia-mesh repair applications, however, these materials were found to have two drawbacks. First, the in vivo degradation rates were too still too rapid, which led to lower mechanical properties than desired. Second, the physical crosslinking of the branched L-valine based PEUs led to solubility challenges for future processability for hernia mesh applications.

Previously, L-phenylalanine based PEUs have been used successfully in bone regeneration applications. The degradation rates of L-phenylalanine based PEUs are slower than what was observed in L-valine based PEUs because the hydrophobic aromatic groups repel water; a major contributor to hydrolytic degradation. Delayed degradation correlated to sustained mechanical properties over a longer period of time. To combine the tunable mechanical properties and limited inflammatory response with sustained mechanical properties and delayed degradation, a series of copolymers consisting of L-valine and L-phenylalanine monomers were synthesized (See, Scheme 4, below). In vitro mechanical properties for this series of copolymers mimic those of poly(propylene) for hernia-mesh repair. In various embodiments, the L-valine-co-L-phenylalanine PEUs of the present invention have also been shown to have improved processability compared to the branched L-valine based PEUs counterparts. Further, the solubility of the polymers in relatively benign solvents (i.e. acetone) allows them to be successfully processed for films through compression molding and blade-coating. This is ideal as these polymers can be cast as standalone films or in combination with ECM to make composite films. Based on mechanical properties from uniaxial tensile tests and burst-tests on PEU-ECM copolymer films and free-standing films, L-valine-co-L-phenylalanine PEUs have shown to be promising materials for hernia mesh applications.

In one or more of these of these embodiments, the amino acid-based poly(ester urea) used to form the implantable polymer mesh of the present invention will comprise a L-valine-co-L-phenylalanine PEU having from about 0.05 mol % to about 0.30 mol %, and preferably from about 0.20 mol % to about 0.30 mol %, L-phenylalanine residues. In some of these embodiments, the L-valine-co-L-phenylalanine PEUs will comprise about 30 mol % L-phenylalanine residues. Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise a L-valine-co-L-phenylalanine PEU having s number average molecular weight (Mₙ) of from about 10,000 g/mol to about 80,000 g/mol, and is preferably from about 40,000 g/mol to about 72,000 g/mol, as measured by size exclusion chromatography (SEC). Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise a L-valine-co-L-phenylalanine PEU polymer having a weight average molecular weight (M_{w}) of from about 20,000 g/mol to about 160,000 g/mol, preferably from about 50,000 g/mol to about 130,000 g/mol and more preferably from about 80,000 g/mol to about 110,000 g/mol, as measured by size exclusion chromatography (SEC). Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise a L-valine-co-L-phenylalanine PEU having a molar mass distributions (Ðₘ) of from about 1 to about 3, preferably from about 1 to about 2, and more preferably from about 1.2 to about 1.9, as measured by size exclusion chromatography (SEC). Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise a L-valine-co-L-phenylalanine PEU having a glass transition temperatures (T_{g}) of from 28° C. to about 60° C., as measured by differential scanning calorimetry. In some of these embodiments, the amino acid-based poly(ester urea) used to form the implantable polymer mesh of the present invention will comprise a L-valine-co-L-phenylalanine PEU having a glass transition temperatures (T_{g}) of from about 35° C. to about 58° C., as measured by differential scanning calorimetry. Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise a L-valine-co-L-phenylalanine PEU having a degradation temperature (T_{d}) of from about 200° C. to about 350° C., as measured by thermogravimetric analysis. In some embodiments, the amino acid-based poly(ester urea) used to form the implantable polymer mesh of the present invention will comprise a L-valine-co-L-phenylalanine PEU having a degradation temperature (T_{d}) of from about 270° C. to about 350° C., as measured by thermogravimetric analysis. Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise a L-valine-co-L-phenylalanine PEU having a Young's modulus of from about 10 MPa to about 500 MPa, as measured by uniaxial tensile testing. In some embodiments, the amino acid-based poly(ester urea) used to form the implantable polymer mesh of the present invention will comprise a L-valine-co-L-phenylalanine PEU having a Young's modulus of from about 50 MPa to about 340 MPa, as measured by uniaxial tensile testing. Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise a L-valine-co-L-phenylalanine PEU having a yield stress (σ_{y}) of from about 2 MPa to about 100 MPa, as measured by uniaxial tensile testing. In some embodiments, the amino acid-based poly(ester urea) used to form the implantable polymer mesh of the present invention will comprise a L-valine-co-L-phenylalanine PEU having a yield stress (σ_{y}) of from about 4 MPa to about 50 MPa, as measured by uniaxial tensile testing. Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise a L-valine-co-L-phenylalanine PEU having a yield strain (ε_{y}) of from about 1% to about 50%, as measured by uniaxial tensile testing. In some embodiments, the amino acid-based poly(ester urea) used to form the implantable polymer mesh of the present invention will comprise a L-valine-co-L-phenylalanine PEU having a yield strain (ε_{y}) of from about 0.1 MPa to about 0.2 MPa, as measured by uniaxial tensile testing. Here, as well as elsewhere in the specification and claims, individual range values can be combined to form additional non-disclosed ranges.

In one or more of these of these embodiments, the amino acid-based poly(ester urea) used to form the implantable polymer mesh of the present invention will comprise an L-valine-co-L-phenylalanine PEU having water uptake from about 0 mass % to about 50 mass %, as measured by phosphine buffered saline solution (PBS) swelling/water uptake testing. Here, as well as elsewhere in the specification and claims, individual range values can be combined to form additional non-disclosed ranges.

In various embodiments, the amino acid-based poly(ester urea) used to form the implantable polymer mesh of the present invention said amino acid-based poly(ester urea) is degradable within the body of a patient. As used herein, the terms “degradable,” and “biodegradable” are used interchangeably to refer to a macromolecule or other polymeric substance susceptible to degradation by biological activity by lowering the molecular masses of the macromolecules that form the substance. Similarly, the term “resorbable” is used herein to refer to a “degradable” or “biodegradable” material, the degradation by products of which are non-toxic to the body and can be removed through ordinary biological processes. As set forth above, the amino acid-based poly(ester urea) used to form the implantable polymer mesh of the present invention are both degradable and resorbable. The ester and urea bonds of the amino acid-based poly(ester urea) used to form the implantable polymer mesh of the present invention allow for both hydrolytic and enzymatic degradation. The final degradation byproducts are amino acids, small diol or polyol segments and CO₂, which can be readily metabolized and/or removed by the body. Moreover, unlike the acidic degradation byproducts of polyesters, the carboxyl group in PEU is buffered by the urea linkages at each repeat unit, reducing or eliminating inflammation in vivo with PEU polymers due, at least in part, to the absence of localized acidification during and after PEU degradation.

As set forth above, in one or more embodiments, the degradable polymer mesh of the present invention may be formed from an implantable composite comprising the amino acid-based poly(ester urea) polymers described above and an extracellular matrix (ECM). As will be understood by those of ordinary skill in the art, the term “extracellular matrix” (ECM) generally refers to the three-dimensional network of extracellular macromolecules, such as collagen, enzymes, and glycoproteins, which provides structural and biochemical support for the cells making up a particular type of tissue, and in the context of the present invention, to such a matrix which has been isolated from the tissue in which it was formed by removal of the cells within the matrix. In various embodiments, these ECMs may comprise decellularized bovine, porcine, or ovine intestinal tissue. In one or more embodiments, the ECM used herein may comprise many sheets of decellularized animal tissue laminated together for additional thickness and strength. The ECM selected is not particularly limited, and may include without limitation, 2.0 1-1 LL SIS-ECM, 2.0 4-LL SIS ECM, Blanket 2 LVP SIS ECM, or Blanket 4 LVP SIS ECM (Cook Biotech Incorporated, West Layfaette, Ind.).

In various embodiments, the degradable polymer mesh of the present invention will comprise an implantable PEU/ECM composite, as described above, having a Young's modulus of from about 10 MPa to about 500 MPa, as measured by uniaxial tensile testing. In some embodiments, the degradable polymer mesh of these embodiments of the present invention will have a Young's modulus of from about 10 MPa to about 500 MPa, in other embodiments, from about 10 MPa to about 400 MPa, in other embodiments, from about 10 MPa to about 300 MPa, in other embodiments, from about 10 MPa to about 200 MPa, in other embodiments, from about 100 MPa to about 500 MPa, in other embodiments, from about 200 MPa to about 500 MPa, in other embodiments, from about 300 MPa to about 500 MPa, and in other embodiments, from about 400 MPa to about 500 MPa, as measured by uniaxial tensile testing. In some embodiments, the degradable polymer mesh of the present invention will comprise a PEU/ECM implantable composite as described above, having a Young's modulus of from about 14 MPa to about 32 MPa, as measured by uniaxial tensile testing. Here, as well as elsewhere in the specification and claims, individual range values can be combined to form additional non-disclosed ranges.

In various embodiments, the degradable polymer mesh of the present invention will comprise a PEU/ECM implantable composite as described above, having a yield stress (σ_{y}) of from about 1 MPa to about 100 MPa as measured by uniaxial tensile testing. In some of these embodiments, the degradable polymer mesh of the present invention will comprise a PEU/ECM composite as described above, having a yield stress (σ_{y}) of from about 1 MPa to about 100 MPa, in other embodiments, from about 1 MPa to about 90 MPa, in other embodiments, from about 1 MPa to about 80 MPa, in other embodiments, from about 1 MPa to about 70 MPa, in other embodiments, from about 1 MPa to about 60 MPa, in other embodiments, from about 10 MPa to about 100 MPa, in other embodiments, from about 20 MPa to about 100 MPa, and in other embodiments, from about 30 MPa to about 100 MPa, as measured by uniaxial tensile testing. In some of these embodiments, the degradable polymer mesh of the present invention will comprise a PEU/ECM implantable composite as described above, having a yield stress (σ_{y}) of from about 44 MPa to about 140 MPa as measured by uniaxial tensile testing. Here, as well as elsewhere in the specification and claims, individual range values can be combined to form additional non-disclosed ranges.

In various embodiments, the degradable polymer mesh of the present invention will comprise a PEU/ECM implantable composite as described above, having a yield strain (ε_{y}) of from about 2% to about 50% as measured by uniaxial tensile testing. In some of these embodiments, the degradable polymer mesh of the present invention will comprise a PEU/ECM composite as described above, having a yield strain (ε_{y}) of from about 2% to about 40%, in other embodiments, from 2% to 30%, in other embodiments, from 2% to 20%, in other embodiments, from 5% to 50%, in other embodiments, from 10% to 50%, in other embodiments, from 20% to 50%, and in other embodiments from 30% to 50%, as measured by uniaxial tensile testing. In some embodiments, the degradable polymer mesh of the present invention will comprise a PEU/ECM implantable composite as described above, having a yield strain (ε_{y}) of from about 1.5 mm/mm to about 6.8 mm/mm as measured by uniaxial tensile testing. Here, as well as elsewhere in the specification and claims, individual range values can be combined to form additional non-disclosed ranges.

In various embodiments, the degradable polymer mesh of the present invention will comprise a PEU/ECM composite as described above, having a force at break from about 30 N to about 300 N as measured by burst testing. In some of these embodiments, the degradable polymer mesh of the present invention will comprise a PEU/ECM composite as described above, having a force at break from about 50 N to about 500 N, in other embodiments, from 100 N to 500 N, in other embodiments, from 200 N to 500 N, in other embodiments, from 300 N to 500 N, in other embodiments, from 30 N to 400 N, in other embodiments, from 30 N to 300 N, and in other embodiments, from 30 N to 200 N, as measured by burst testing. In some embodiments, the degradable polymer mesh of the present invention will comprise a PEU/ECM composite as described above, having a force at break from about 93 N to about 162 N as measured by burst testing. Here, as well as elsewhere in the specification and claims, individual range values can be combined to form additional non-disclosed ranges.

In various embodiments, the degradable polymer mesh of the present invention will comprise a PEU/ECM composite as described above, having an extension at break from about 0.5 cm to about 5 cm as measured by burst testing. In some embodiments, the degradable polymer mesh of the present invention will comprise a PEU/ECM composite as described above, having an extension at break from about 0.5 cm to about 5 cm, in other embodiments, from 0.5 cm to 4 cm, in other embodiments, from 0.5 cm to 3 cm, in other embodiments, from 0.5 cm to 2 cm, in other embodiments, from 1 cm to 5 cm, in other embodiments, from 2 cm to 5 cm, and in other embodiments, from 3 cm to 5 cm, as measured by burst testing. In these embodiments, the degradable polymer mesh of the present invention will comprise a PEU/ECM composite as described above, having an extension at break from about 1.0 cm to about 2.4 cm as measured by burst testing. Here, as well as elsewhere in the specification and claims, individual range values can be combined to form additional non-disclosed ranges.

In various embodiments, the degradable polymer mesh of the present invention will comprise a PEU/ECM composite as described above, having a relative stiffness from about 25 N/cm to about 200 N/cm as measured by burst testing. In some embodiments, the degradable polymer mesh of the present invention will comprise a PEU/ECM composite as described above, having a relative stiffness from about 25 N/cm to about 150 N/cm, in other embodiments, from 25 N/cm to 100 N/cm, in other embodiments, from 25 N/cm to 50 N/cm, in other embodiments, from 50 N/cm to 200 N/cm, in other embodiments, from 75 N/cm to 200 N/cm, in other embodiments, from 100 N/cm to 200 N/cm, and in other embodiments, from 150 N/cm to 200 N/cm, as measured by burst testing. In some embodiments, the degradable polymer mesh of the present invention will comprise a PEU/ECM composite as described above, having a relative stiffness from about 60 N/cm to about 95 N/cm as measured by burst testing. Here, as well as elsewhere in the specification and claims, individual range values can be combined to form additional non-disclosed ranges.

In various embodiments, the degradable polymer mesh of the present invention will comprise a PEU/ECM composite as described above, having a water uptake from about 0 mass % to about 45 mass % as measured by phosphine buffered saline solution (PBS) swelling/water uptake measurements. In some of these embodiments, the degradable polymer mesh of the present invention will comprise a PEU/ECM composite as described above, having a water uptake from about 0 mass % to about 45 mass %, in other embodiments, from about 10 mass % to about 50 mass %, in other embodiments, from about 20 mass % to about 50 mass %, in other embodiments, from about 30 mass % to about 50 mass %, in other embodiments, from about 0 mass % to about 40 mass %, in other embodiments, from about 0 mass % to about 30 mass %, and in other embodiments, from about 0 mass % to about 20 mass %, as measured by phosphine buffered saline solution (PBS) swelling/water uptake testing. Here, as well as elsewhere in the specification and claims, individual range values can be combined to form additional non-disclosed ranges.

In various embodiments, the polymer mesh of the present invention will elicits less inflammatory response than polymer mesh formed from polypropylene, as measured by hematoxylin and eosin histological fibrous capsule measurements. In one or more embodiments, the polymer mesh of the present invention will have a elicit less fibrous capsule formation than polymer mesh for soft tissue repair formed from polypropylene, as measured by hematoxylin and eosin histological fibrous capsule measurements.

As set forth above, once the amino acid-based poly(ester urea) polymers described above are synthesized, they may be formed into the implantable polymer mesh of various embodiments of the present invention by any suitable means including, without limitation, compression molding, vacuum molding, blade coating, flow coating, electrospinning, melt blowing, or solvent casting. In one or more embodiment, the implantable polymer mesh of the present invention may be formed from the amino acid-based poly(ester urea) polymers described above as follows. In these embodiments, the amino acid-based poly(ester urea) polymer is first pulverizing to a powder in a grinder and placed in a mold configured for use in a vacuum compression molding device and defining the desired 3-dimensional shape implantable polymer mesh. The amino acid-based poly(ester urea) polymer powder is then heated in the mold to a temperature above its melting temperature (Tₘ) and below its degradation temperature (T_{d}) to melt said amino acid-based poly(ester urea) polymer powder and compressing into the mold with a force of from about 20 MPa to about 200 MPa. The mold is then allowed to cool to ambient temperature to produce the implantable polymer mesh of the present invention.

The method for forming the implantable polymer mesh of the present invention from the amino acid based PEU polymers or PEU/ECM composites described above is not particularly limited and any suitable method known in the art for that purpose may be used. In one or more embodiment, the implantable polymer mesh of the present invention may be formed by first forming a polymer film or sheet and then perforating it to form a mesh. In some these embodiments, the implantable polymer mesh of the present invention may be formed by first forming a polymer film or sheet of one of the amino acid based PEU polymers described above by blade coating, flow coating, or solvent casting, and then perforated to form the mesh. In some other of these embodiments, the implantable polymer mesh of the present invention may be formed by first forming a PEU/ECM composite film or sheet by forming a polymer film or sheet of one of the amino acid based PEU polymers described above onto an ECM sheet by blade coating, flow coating, or solvent casting, and then perforated the resulting PEU/ECM composite film or sheet to form the mesh. In some other embodiments, the implantable polymer mesh of the present invention may be formed by electrospinning or melt blowing the amino acid based PEU polymer into a non-woven polymer mesh. In some other embodiments, an ECM mesh may be formed using conventional methods and the amino acid based PEU polymers discussed above added by dip coating or spin coating the ECM mesh with the amino acid based PEU polymer as described above.

As set forth above, in some embodiments, the implantable polymer mesh of the present invention may be formed from a PEU/ECM composite as set forth above, using a blade coating, flow coating, or solvent casting device. In one or more of these embodiments, the amino acid-based poly(ester urea) polymer is first dissolved in a suitable solvent or solvent solution. Next, an extracellular matrix (ECM) is fastened to a substrate suitable for use in a blade coating, flow coating, or solvent casting device to form an ECM/substrate combination, which is configured for use in a blade coating, flow coating, or solvent casting device. The dissolved amino acid-based poly(ester urea) polymer solution is then fed into a solution well that is configured for use with the blade coating, flow coating, or solvent casting device and the solution well is then secured to the blade coating, flow coating, or solvent casting device. The ECM/substrate combination is then moved through the blade coating, flow coating, or solvent casting device at a velocity from about 0 cm/s to about 200 cm/s, during which time a layer of PEU polymer is applied to form a PEU/ECM composite with extracellular matrix acting as the substrate for the PEU attachment. Next, the solvent is removed from the poly(ester urea) polymer coated on the extracellular matrix (PEU/ECM composite) by drying at ambient temperatures from about 20° C. to about 35° C. for a time from about 1 hour to about 24 hours. The PEU/ECM composite is then placed under vacuum pressure from about 5 mm/Hg to about 25 mm/Hg for a time from about 1 hour to about 24 hours to remove any residual solvent and removed from the substrate to provide the desired PEU/ECM composite mesh.

### EXPERIMENTAL

In order to more fully illustrate and further reduce the amino acid based poly(ester urea) polymers of the implantable polymer mesh of the present invention to practice, the following experiments were conducted. In a first set of experiments, the mechanical properties of a series of L-valine based poly(ester urea)s PEUs having a variety of monomer diol lengths and the degree of branching were assessed in vitro and in vivo. In a second set of experiments, a series of L-valine-co-L-phenylalanine poly(ester urea) copolymers with desirable mechanical properties for soft-tissue repair applications were investigated.

### I. Preclinical In Vitro and In Vivo Assessment of Linear and Branched L-Valine Based Poly(Ester Urea)s for Soft-Tissue Applications.

A series of L-valine based poly(ester urea)s PEUs were fabricated that possessed a variation in monomer diol length and the extent of branching. The resulting Young's moduli (104.9±30.4-268.6±11.8 MPa) are comparable to currently employed poly(propylene) (164.9±4.7 MPa) materials for medical applications in hernia-mesh repair. A 2% branched poly(1-VAL-8) maintained the highest Young's modulus following 3 month in vivo implantation (77.8±34.1 MPa) when compared to other PEU analogs (20.4±5.5-45.3±5.3 MPa). Both the linear and branched PEUs elicited a limited inflammatory response in vivo as manifested in significantly less fibrous capsule formation after 3 months of implantation (80.3±37.8-103.0±32.7 m) when compared to the poly(propylene) controls (126.2±34.1 μm). Mechanical degradation in vivo through 3 months coupled with limited inflammatory response make L-valine based PEUs a viable material for soft-tissue applications.

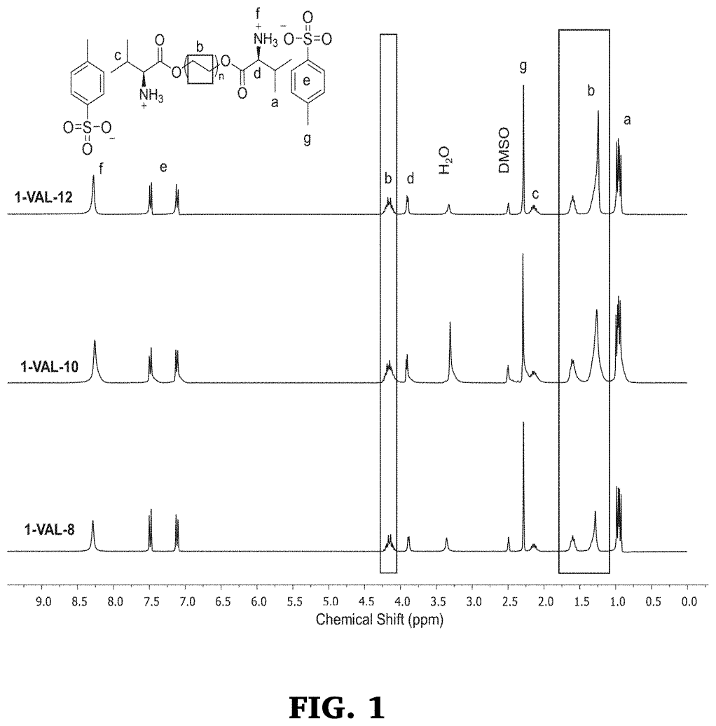

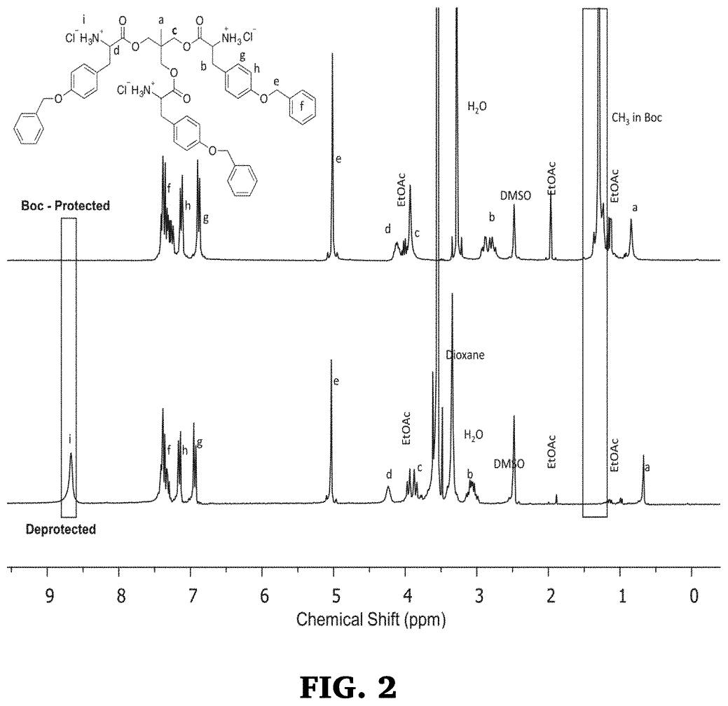

Synthesis: All linear and branched monomers were synthesized and characterized using ¹H-NMR spectroscopy (FIGS. 1, 2). (See, Examples 1-6). An esterification with 1,8-octanediol, 1,10-decanediol, and 1,12-dodecanediol with the carboxylic acid of L-valine using p-toluenesulfonic acid to prevent reactions at the amine moiety. The three monomers were named accordingly to their diol-chain length (1-VAL-8) formed from 1,8-octanediol, (1-VAL-10) formed from 1,10-decanediol, and (1-VAL-12) formed from 1,12-dodecanediol. The linear monomers have similar ¹H NMR resonances with the only variation coming from the integration that corresponds to the methylene peaks in the varying diol-chain lengths, shown between 1.22-1.35 ppm. Thus, the successful synthesis for each linear monomer was confirmed. The branched monomer was synthesized via an esterification reaction between 1,1,1-tri(hydroxylmethyl)ethane and Boc-O-benzyl-L-tyrosine using DIC as the coupling reagent. Unwanted urea byproducts were removed via a silica gel filtration and the final product was afforded following Boc-deprotection with 4 M HCl/dioxane, as demonstrated by the disappearance of the singlet at 1.28 ppm and appearance of the broad amine peak at 8.72-8.78 (ppm).

The PEUs were polymerized using (1-VAL-8), (1-VAL-10), and (1-VAL-12) with triphosgene using an interfacial reaction (See Scheme 3, below).

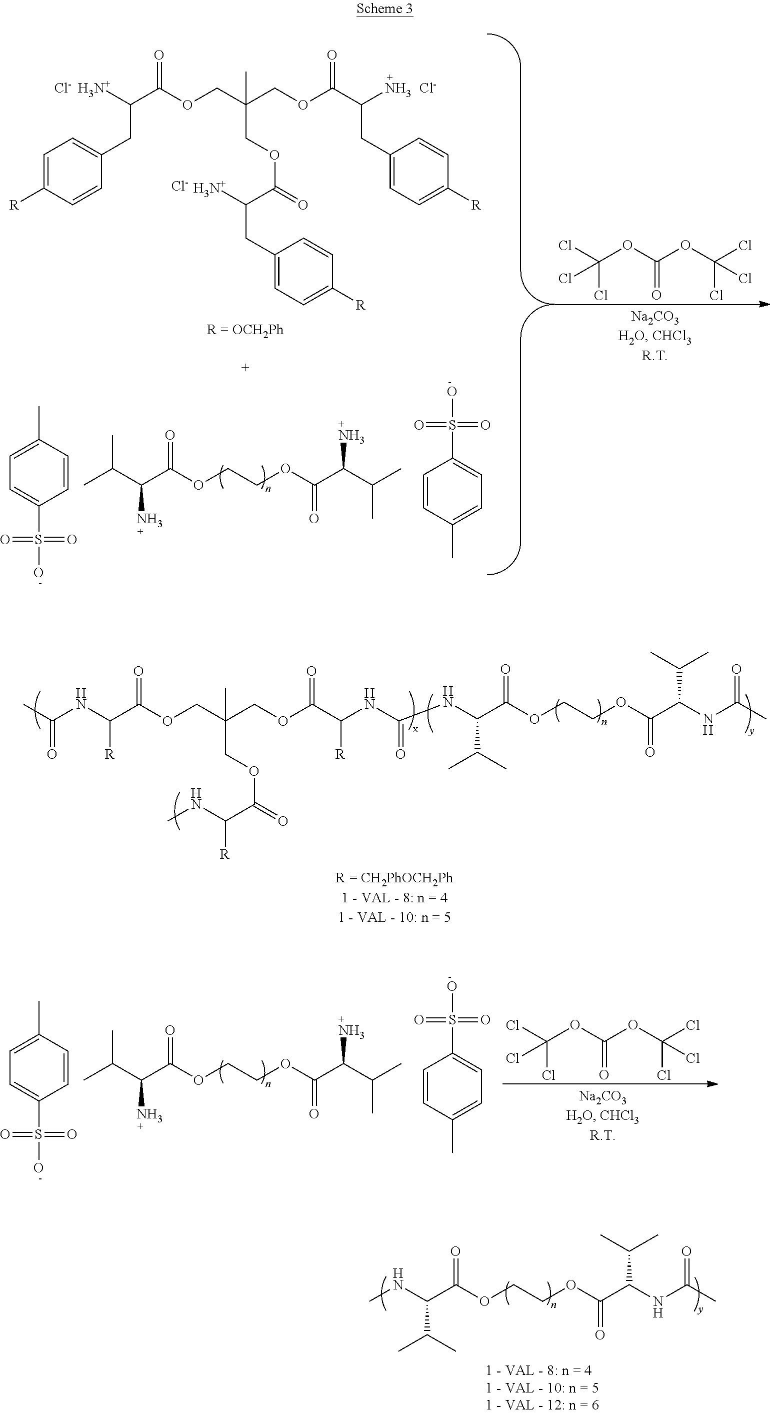

Scheme 3 shows a general synthetic scheme for L-valine monomers with diol-chain lengths varied between 8, 10, and 12 methylene units. Poly(1-VAL-8), poly(1-VAL-10), and poly(1-VAL-12) were synthesized using interfacial polymerization with triphosgene. In these experiments, branched PEUs were synthesized using 1-VAL-8 and 1-VAL-10 with a 2% molar feed ratio of Triol-TYR to afford 2% branched poly(1-VAL-8) and 2% branched poly(1-VAL-10), respectively.

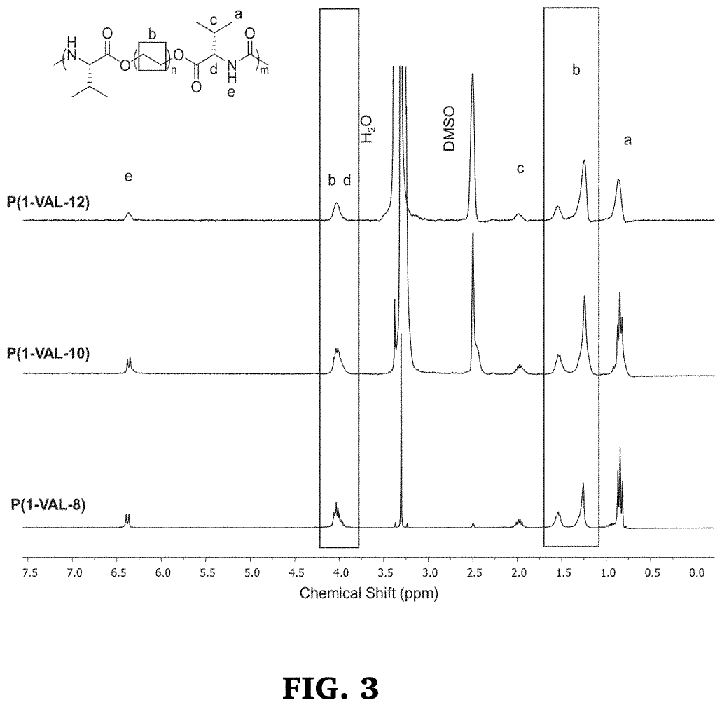

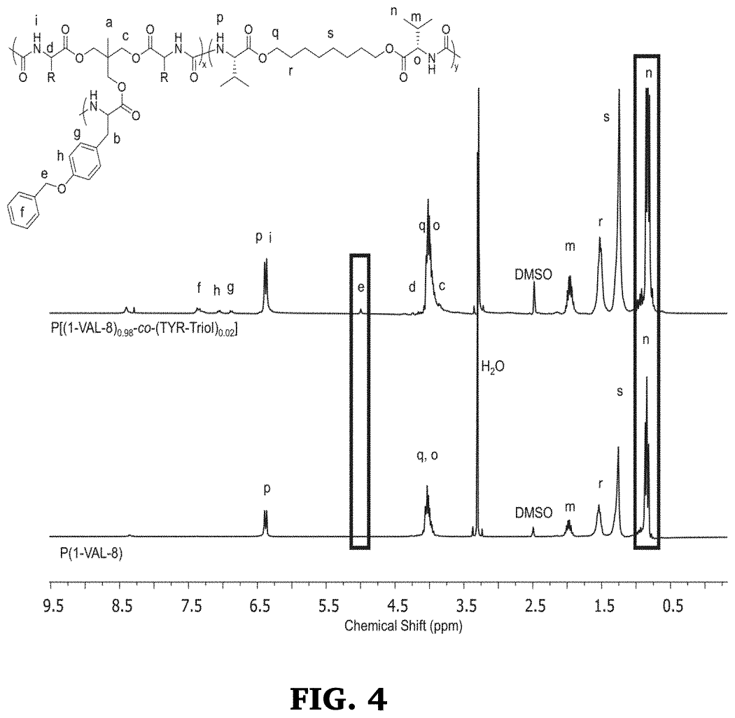

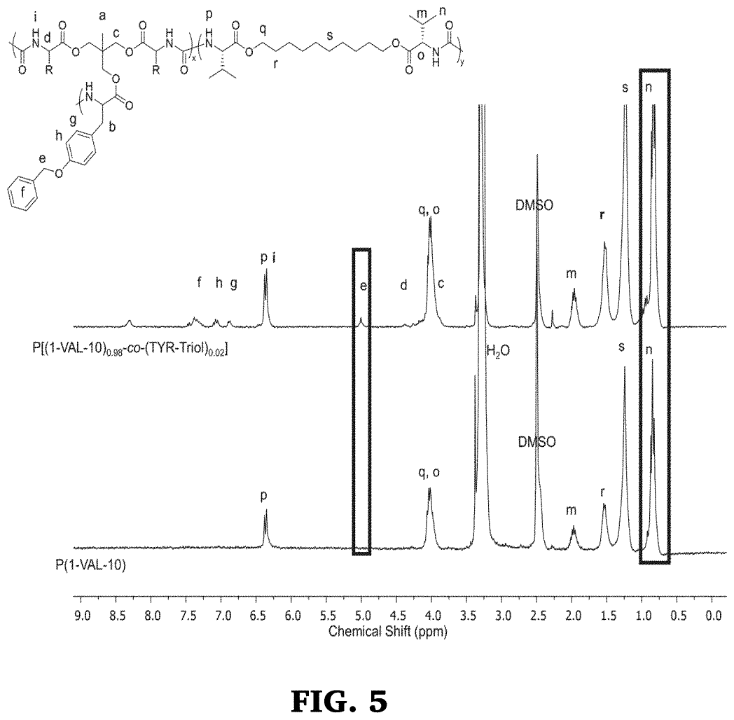

The L-valine amino acid was chosen for its less rigid side chain when compared previously studied L-phenylalanine and L-tyrosine which would afford greater chain flexibility. Polymer synthesis was confirmed through ¹H-NMR (FIG. 3). The polymers spectra are discernible from the variable intensity of the methylene resonances highlighted and denoted “b”. Post precipitation yields were 82-92%. Branched PEUs were prepared using (1-VAL-8) or (1-VAL-10) with (Triol-TYR) (Scheme 3) in a molar feed ratio of 98:2 respectively. Successful synthesis was confirmed through ¹H-NMR (FIGS. 4, 5). The extent of branching was determined by comparing integration of the six methylene protons denoted “e” from the Triol-TYR monomer and comparing them to the twelve methyl L-valine protons denoted “n” from the linear monomers. Post precipitation yields were 79-88% for 2% branched poly(1-VAL-8) and 2% branched poly(1-VAL-10) respectively. Post precipitation molecular mass and molecular mass distributions for all five polymers are listed in Table 1, below.

TABLE 1
Physical properties of poly(1-VAL-8), poly(1-VAL-10),
poly(1-VAL-12), poly[(1-VAL-8)_{0.98}-co-(Triol-TYR)_{0.02}], and
poly[(1-VAL-10)_{0.98}-co-(Triol-TYR)_{0.02}] polymers

| | T_{g} | T_{d} | Mₙ | M_{w} | |
|---|---|---|---|---|---|
| Polymer | (° C.) | (° C.) | (kDa) | (kDa) | Ðₘ |
| P(1-VAL-8) | 42 | 310 | 42 | 71 | 1.7 |
| P(1-VAL-10) | 34 | 339 | 46 | 71 | 1.6 |
| P(1-VAL-12) | 29 | 205 | 51 | 75 | 1.4 |
| 2% Branched P(1-VAL-8) | 35 | 301 | 126 | 410 | 3.3 |
| 2% Branched P(1-VAL-10) | 31 | 320 | 68 | 137 | 2.0 |
| Poly(propylene) | — | 384 | — | — | — |

The physical properties of L-valine PEUs were assessed prior to sterilization and in vivo implantation. The T_{g}, T_{d}, Mₙ, M_{w}, and Dm were all recorded.

All M_{w} values are greater than 71 kDa with Dm 1.7-3.3. Linear PEUs have Ðₘ less than the theoretical value 2.0 because the lower molecular mass chains are lost during the precipitation process. The 2% branched polymers exhibit higher molecular mass because the Mₙ and M_{w} values were obtained from a linear polystyrene standard.

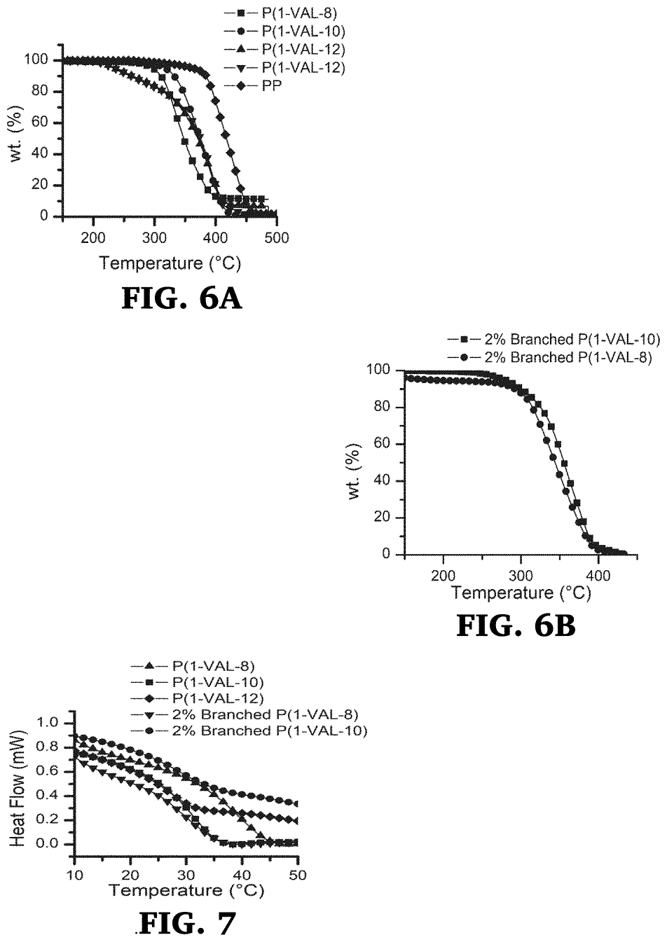

Physical Properties: Thermal gravimetric analyses (TGA) for linear PEUs and 2% branched PEUs (FIGS. 6A-B) show the high degradation temperatures when compared to compression molding processing temperatures. Poly(1-VAL-12) shows a broader degradation temperature which is consistent with previously published work and could be attributed to greater flexibility which allows for more degradation processes. Values of T_{d} are significantly higher than the reported T_{g} values (FIG. 7). The change in diol chain length affected the T_{g} with values ranging between 29-42° C. (See, Table 1). When the branching unit is incorporated, a drop in the T_{g} is observed. This can be attributed to the branching unit interrupting interchain packing and hydrogen bonding between the urea groups.

In Vivo Degradation:

In vivo polymer tensile bar implantation was performed using melt pressed ASTM standard tensile bars (see, American Society for Testing and Materials (ASTM) standard ASTM D638-614 (2014), the disclosure of which is incorporated herein by reference it its entirety), which were cut with a dye-cutter then placed subcutaneously into the backs of female Sprague-Dawley rats. See Example 8, below. A small incision was made with a surgical blade followed by subcutaneous pocket tunneling with hemostats, leading to polymer implantation and final incision closure with Michel clips. Tracking molecular mass degradation from sterilization to in vivo implantation is important as mechanical failure in any soft-tissue device is likely to accompany molecular mass degradation (See, Table 2, below).

TABLE 2
Molecular mass degradation determined by SEC*

| | Initial | | | Post-EtO | | | 2 month | | | 3 Month | | |
|---|---|---|---|---|---|---|---|---|---|---|---|---|
| Polymer | Mₙ | M_{w} | Ðₘ | Mₙ | M_{w} | Ðₘ | Mₙ | M_{w} | Ðₘ | Mₙ | M_{w} | Ðₘ |
| P(1-VAL-8) | 42 | 71 | 1.7 | 53 | 79 | 1.5 | 59 | 79 | 1.3 | 94 | 105 | 1.1 |
| P(1-VAL-10) | 46 | 71 | 1.6 | 46 | 67 | 1.4 | 36 | 61 | 1.7 | 96 | 106 | 1.1 |
| P(1-VAL-12) | 51 | 75 | 1.4 | 59 | 78 | 1.3 | 66 | 85 | 1.2 | 102 | 111 | 1.1 |
| 2% Branched P(1-VAL-8) | 126 | 410 | 3.3 | 61 | 113 | 1.9 | 65 | 247 | 3.8 | 67 | 210 | 3.1 |
| 2% Branched P(1-VAL-10) | 68 | 137 | 2.0 | 63 | 150 | 2.4 | 44 | 94 | 2.2 | 46 | 85 | 1.9 |

| | | | | |
|---|---|---|---|---|
| Polypropylene | — | — | — | — |

*Molecular masses were measured using SEC for the polymers before and after EtO sterilization and at each time point. The Mₙ, M_{w}, and Ðₘ are reported.

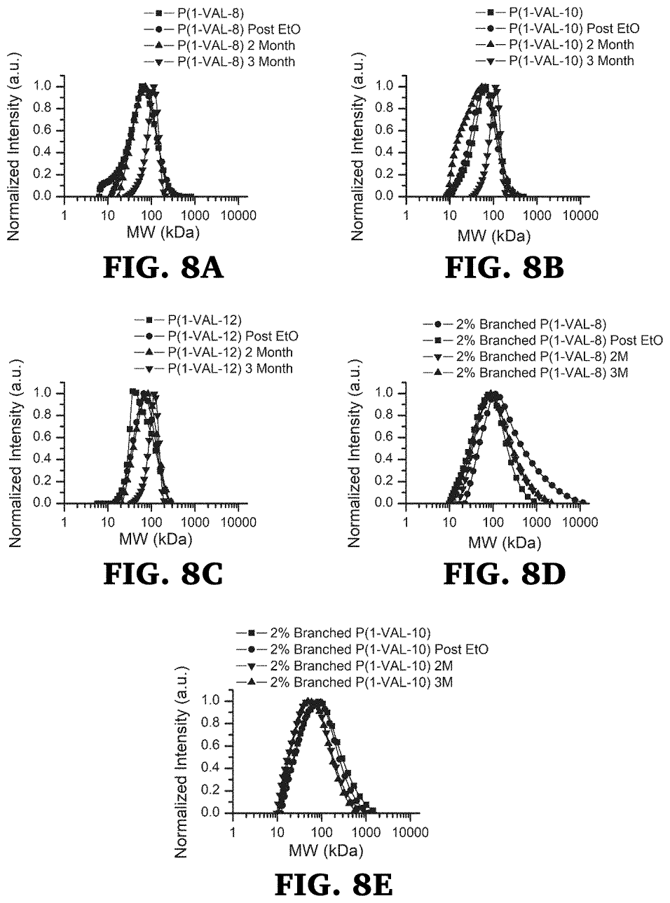

Molecular masses were measured using SEC (FIGS. 8A-E). The molecular masses of the PEUs were maintained throughout ethylene oxide (EtO) sterilization which is promising for the commercialization of these materials in soft-tissue repair applications. An increase in Mₙ and M_{w} values is observed post-implantation. This change is attributed to lower molecular weight polymer chains that have greater mobility and allows for greater hydrolysis infiltration and subsequent degradation leaving heavier chains behind and narrower Ðₘ. This trend holds when comparing the poly(1-VAL-8), poly(1-VAL-10), and poly(1-VAL-12) molecular weight values. The molecular weights for the 2% branched polymers shows less of a clear trend, however, the molecular weight distribution does narrow between the initial and 3 month time points likely due to the degradation of shorter chains.

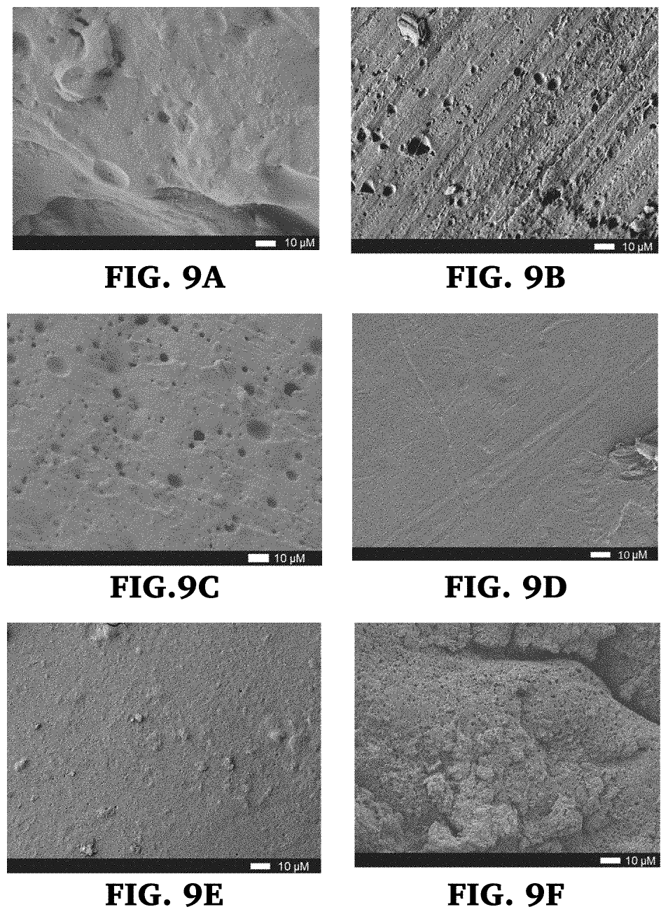

Surface topology images of the PEUs and poly(propylene) (FIGS. 9A-F) illustrate the in vivo degradation post implantation. All PEU analogues elicited a surface eroding morphology which is consistent with previously studied PEU materials. Based off of SEM surface morphology, poly(1-VAL-8) (FIG. 9A) showed more cavities and surface defects than poly(1-VAL-10) (FIG. 9B) and poly(1-VAL-12) (FIG. 9C) which displayed intermediate degradation. This was expected as a shorter diol chain length polymer contains more hydrolytically cleavable ester functional groups in the polymer backbone when compared to longer diol chain length analogues. The smooth surface topology of the PP (FIG. 9D) is indicative of the non-resorbable nature and correlates well with the sustained in vivo mechanical degradation results. 2% branched poly(1-VAL-8) (FIG. 9E) showed limited surface erosion when compared to its PEU linear analogue which was consistent with the in vivo mechanical degradation results. Whale to wanting to be bound by theory, it is believed that this result can be attributed to the covalent crosslinking and hydrophobicity of the branching unit which help repel water and subsequent hydrolytic surface erosion. The 2% branched poly(1-VAL-10) (FIG. 9F), did not correlate well with the observed trends from its PEU analogues as it showed the larger cavities and undulations than poly(1-VAL-10). However, this result did correlate well with the observed in vivo mechanical degradation results as 2% poly(1-VAL-10) elicited the greatest amount of in vivo mechanical degradation through 3 months (See Table 3, below).

TABLE 3
Mechanical properties comparison

| Polymer | | Initial | Post-EtO | 2 Month | 3 Month |
|---|---|---|---|---|---|
| P(1-VAL-8) | Modulus (MPa) | 193.4 ± 13.6 | 254.9 ± 10.6 | 70.2 ± 2.9 | 45.3 ± 5.3 |
| | σ_{y} (MPa) | 34.4 ± 2.1 | 37.3 ± 7.7 | 7.2 ± 1.7 | 5.6 ± 1.2 |
| | ε_{y} (min/mm) | 0.2 | 0.2 | 0.1 | 0.1 |
| P(1-VAL-10) | Modulus (MPa) | 183.4 ± 10.7 | 222.5 ± 2.3 | 60.1 ± 38.3 | 37.8 ± 6.1 |
| | σ_{y} (MPa) | 22.2 ± 1.4 | 30.9 ± 0.3 | 7.3 ± 8.0 | 8.6 ± 1.5 |
| | ε_{y} (mm/mm) | 0.1 | 0.2 | 0.1 | 0.2 |
| P(1-VAL-12) | Modulus (MPa) | 104.9 ± 30.4 | 175.0 ± 17.0 | 48.2 ± 23.6 | 39.7 ± 5.1 |
| | σ_{y} (MPa) | 10.3 ± 1.0 | 17.6 ± 1.0 | 3.9 ± 3.3 | 1.1 ± 0.4 |
| | ε_{y} (mm/mm) | 0.2 | 0.1 | 0.1 | <0.1 |
| 2% Branched | Modulus (MPa) | 268.6 ± 11.8 | 251.4 ± 12.3 | 86.4 ± 21.6 | 77.8 ± 34.1 |
| P(1-VAL-8) | σ_{y} (MPa) | 53.0 ± 4.2 | 43.1 ± 4.6 | 14.1 ± 5.8 | 6.5 ± 1.0 |
| | ε_{y} (mm/mm) | 0.2 | 0.2 | 0.2 | 0.1 |
| 2% Branched | Modulus (MPa) | 140.0 ± 71.0 | 195.5 ± 8.8 | 7.7 ± 2.0 | 20.4 ± 5.5 |
| P(1-VAL-10) | σ_{y} (MPa) | 18.2 ± 8.2 | 13.3 ± 8.1 | 0.1 ± <0.1 | 0.1 ± 0.1 |
| | ε_{y} (mm/mm) | 0.2 | 0.1 | <0.1 | <0.1 |
| Polypropylene | Modulus (MPa) | 164.9 ± 4.7 | 194.4 ± 10.0 | 200.3 ± 11.7 | 190.1 ± 8.5 |
| | σ_{y} (MPa) | 27.4 ± 0.2 | 25.4 ± 1.0 | 26.3 ± 1.3 | 22.4 ± 1.9 |
| | ε_{y} (mm/mm) | 0.2 | 0.2 | 0.2 | 0.1 |

The Young's modulus, stress at yield (σy) and strain at yield (εy) were measured and recorded. Values reported are an average of 4-6 samples.

Mechanical Properties:

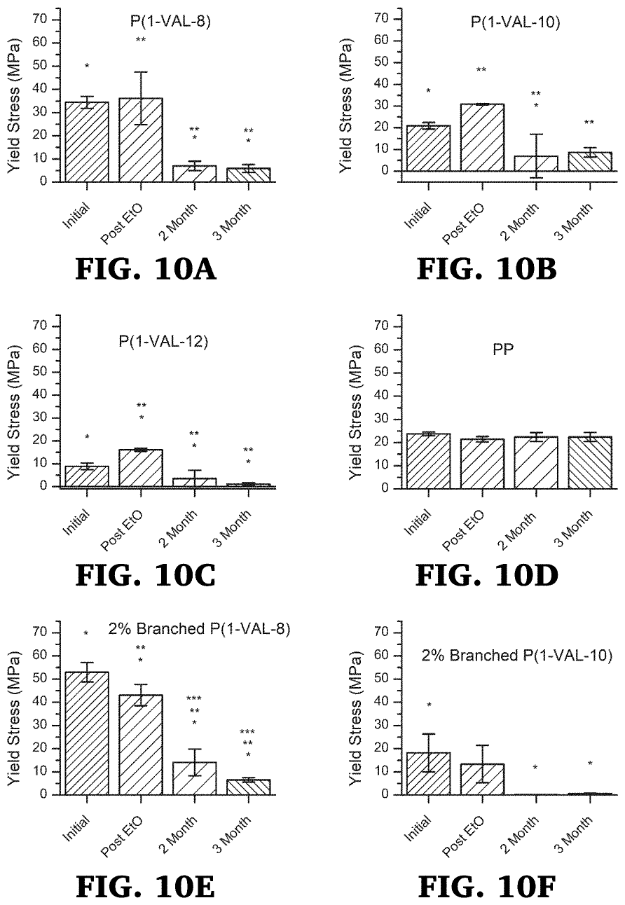

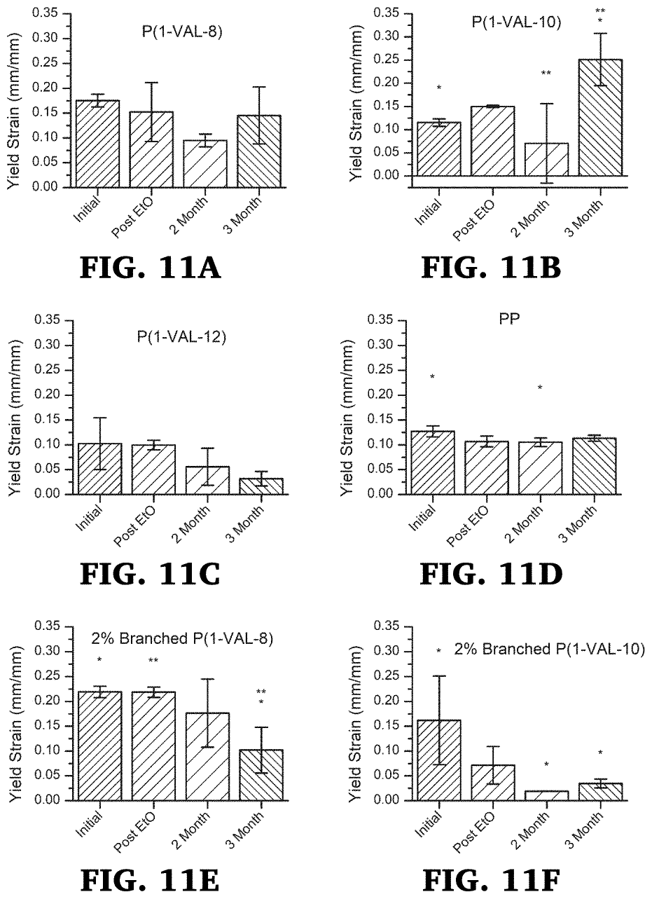

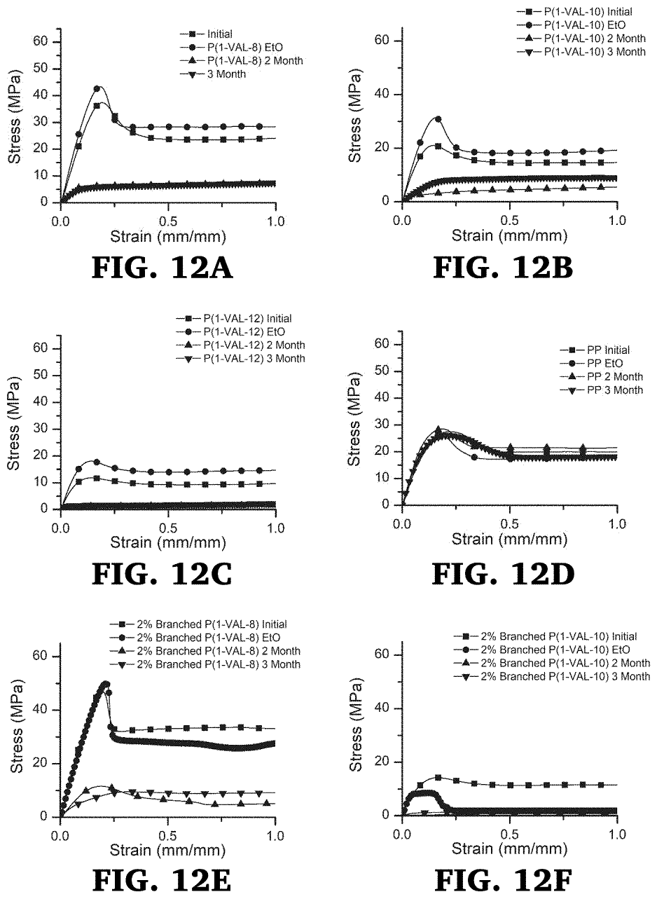

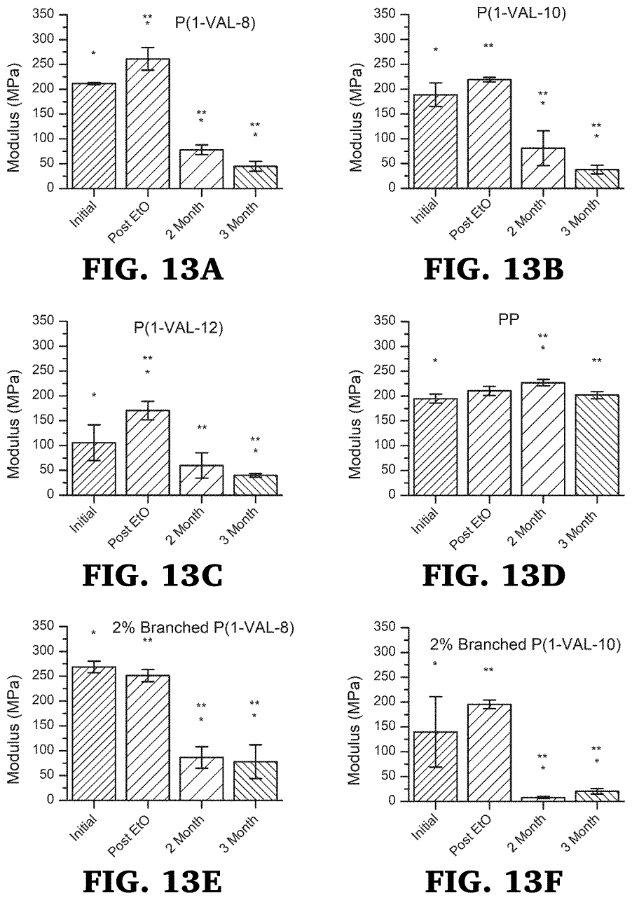

Tensile testing was performed on the PEU and poly(propylene) tensile bars prior to implantation, after sterilization, and at each in vivo time point. See Examples 7-8, below. The stress and strain curves were recorded (see, FIGS. 10-12) and all extrapolated values were reported (Table 3). The Young's modulus was extrapolated from the linear region of the stress and strain curves (see, FIG. 13) and compared before and after implantation. For the linear and branched PEU analogues, a decrease in diol chain length correlated to an increase in Young's modulus. This was expected as increasing diol chain length will increase the polymer chain flexibility and decreases the material stiffness. Once exposed to EtO sterilization poly(1-VAL-8) (FIG. 12A) and poly(1-VAL-12) (FIG. 12C) modulus increased slightly. While not wanting to be bound by theory, it is believed that the increase can be attributed to EtO having a plasticizing effect on the polymers and a corresponding increase in hydrogen bonding among the urea groups. Sustained mechanical properties after sterilization was ideal for the future commercialization of these materials. When comparing sterilized samples to in vivo samples, a modulus drop was observed across all samples except for polypropylene. This drop in modulus is indicative of PEUs hydrolytic and enzymatic degradation in vivo which decreased the stiffness. 2% branched poly(1-VAL-8) (FIG. 12E) was the PEU that maintained the largest modulus through the three-month time point. This was expected as the 2% branched poly(1-VAL-8) has the shortest diol chain length which correlates to enhanced stiffness and a hydrophobic branching unit which slows degradation when compared to the linear counterparts. The yield stress (σ_{y}) and yield strain (ε_{y}) were subsequently measured after the linear region. The σ_{y} (FIGS. 10A-F) trends downward for all PEUs post-implantation. As the polymer chains are hydrolytically and enzymatically cleaved, the σ_{y} naturally decreases as the number of chains with molecular weights above chain entanglement diminishes. This trend was not observed for PP samples as degradation was nominal. The ε_{y} across samples did not show a clear trend (FIGS. 11A-F). For the 2% branched poly(1-VAL-8) (FIG. 11E) samples, a decrease in ε_{y} was observed after three months. Alternatively, poly(1-VAL-10) (FIG. 11B) saw an increasing while with other samples like poly(1-VAL-8) (FIG. 11A) and poly(1-VAL-12) (FIG. 11C) exhibited no change. Variation in ε_{y} was minimal between samples as all samples fell between 0.0-0.2 mm/mm. This indicated that although there was no observable trend, there was a well-defined ε_{y} range for these materials.

Histology:

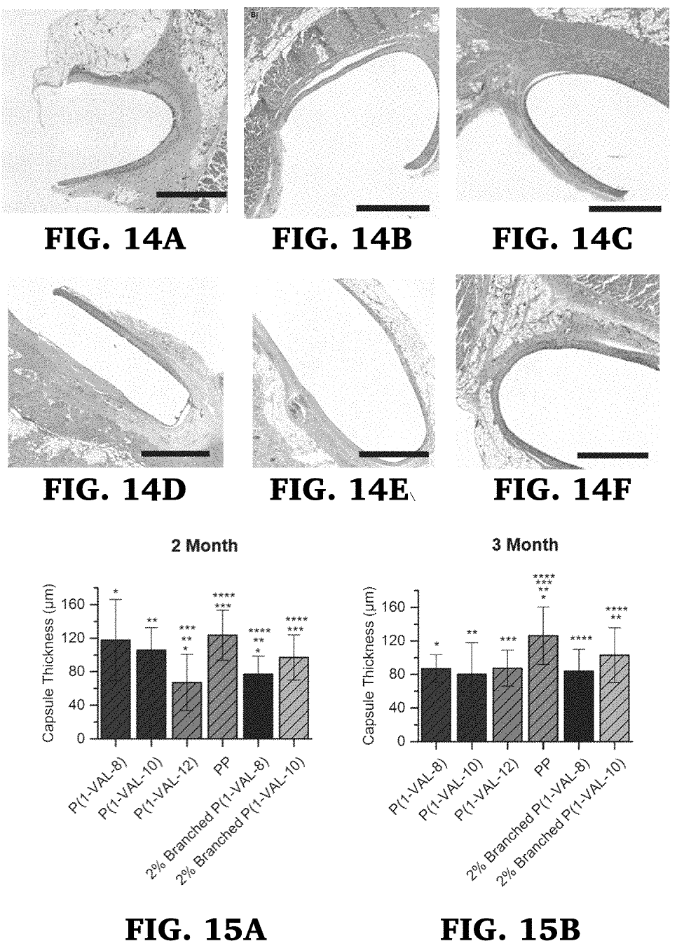

Histology images for PEUs and the poly(propylene) control are shown in FIG. 14. The H&E images are cross-sectional areas of paraffin embedded polymer and surrounding tissue postmortem. See Example 9, below. Implanted biomaterials characteristically induce a foreign body response and elicit a collageneous fibrous capsule as the surrounding cells attempt to wall off the implanted material. Fibrous capsule is an indication of sustained chronic inflammation and a trademark of non-resorbable biomaterials. One of the challenges with non-resorbable polymers as hernia repair materials, is that sustained chronic inflammation prevents tissue remodeling which leads to tissue weakness and ultimate hernia recurrence. The fibrous capsule can be identified as the dark tissue surrounding the polymer implant where cell nuclei are stained dark purple from hematoxylin and tissue is stained pink from eosin. Cell nuclei accumulation is observed in the fibrous capsule and capsule thickness was measured around the perimeter of each polymer implant at 2 and 3 month time points (FIG. 15) with values reported in Table 4.

TABLE 4
Fibrous capsule histology measurements

| Fibrous Capsule | | |
|---|---|---|
| Thickness | 2 Month (μm) | 3 Month (μm) |
| P(1-VAL-8) | 117.7 ± 48.6 | 87.1 ± 16.4 |
| P(1-VAL-10) | 105.7 ± 27.0 | 80.3 ± 37.8 |
| P(1-VAL-12) | 67.3 ± 33.4 | 87.6 ± 21.5 |
| 2% Branched P(1-VAL-8) | 77.0 ± 21.6 | 84.0 ± 25.9 |
| 2% Branched P(1-VAL-10) | 97.0 ± 26.9 | 103.0 ± 32.7 |
| Polypropylene | 123.5 ± 29.8 | 126.2 ± 34.1 |

At 2 months, both branched PEU analogues have different capsule thickness levels than PP however no significant difference is observed for the linear PEUs. At two months, the resorbable nature of PEUs did not have a noticeable effect on fibrous capsule when compared to the non-resorbable PP counterpart. At 3 months however, all five PEUs exhibit smaller fibrous capsule thickness than PP. This change can be attributed to the remodeling process differences between PEUs and PP. As PEUs degrade, cellular infiltration can occur which leads to a shift from chronic inflammation towards tissue remodeling. This shift towards native tissue deposition through the tissue remodeling process is seen from the drop in fibrous capsule thickness. Remodeling is ideal for a hernia repair material as native tissue will have greater mechanical integrity than fibrous capsule scar tissue. The improved inflammatory response over time for the PEU analogues compared to PP makes these materials exciting candidates for soft-tissue applications.

In these experiments, series of L-valine based PEUs were evaluated for hernia-mesh repair applications. These materials showed in vitro Young's moduli comparable to currently employed polymeric materials for medical applications in hernia-mesh repair (104.9±30.4-268.6±11.8 MPa). Of the implanted PEUs, 2% branched poly(1-VAL-8) showed the greatest mechanical properties through in vivo implantation. All of the PEUs resulted in limited inflammatory response through three months when compared to the polypropylene. Limited inflammatory response through 3 months, along with tunable mechanical properties, make these L-valine based PEUs an excellent material for hernia-mesh repair and other soft tissue applications.

### II. In Vitro Assessment of Amino Acid Based Poly(Ester Urea) Copolymers for Hernia-Repair.

In these experiments, a series of L-valine-co-L-phenylalanine poly(ester urea) copolymers with desirable mechanical properties for soft-tissue repair applications were investigated. Poly[(1-VAL-8)₇₀-co-(1-PHE-6)₃₀] showed the highest initial uniaxial mechanical properties (332.5±3.5 MPa). L-valine-co-L-phenylalanine poly(ester urea)s were blade coated on extracellular matrix (ECM) and poly[(1-VAL-8)₉₀-co-(1-PHE-8)₁₀] and poly[(1-VAL-8)₇₀-co-(1-PHE-8)₃₀] enhanced the mechanical properties of ECM in composite films (149.4±7.9 N and 151.4±11.3 N respectively). Free standing films of L-valine-co-L-phenylalanine poly(ester urea)s were found to have superior extension at break when compared to ECM (averages between 1.2-1.9 cm and 1.2 cm respectively). Use of PEU copolymers in soft-tissue repair applications as standalone materials or as composite materials are viable alternatives to currently employed biologic materials for hernia-mesh repair applications.

Synthesis:

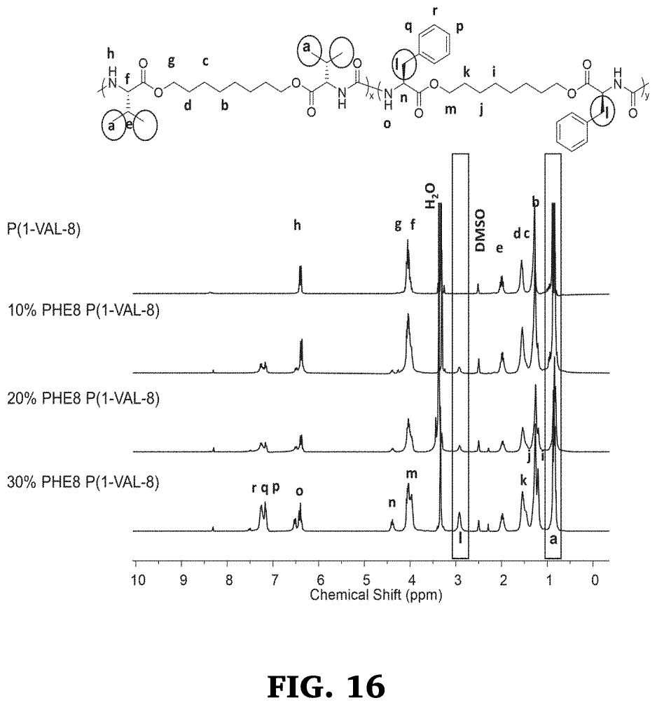

Various amino acid based monomers were synthesized (see Scheme 1, above) and characterized using ¹H-NMR (FIGS. 16-18). (See, Examples 10-13). In particular, 1,6-hexanediol and 1,8-octanediol were coupled to the carboxylic acid of L-valine or L-phenylalanine through an esterification using p-toluenesulfonic acid to prevent amine moiety. The resulting monomers were named based on their diol chain length and amino acid; (1-VAL-8) formed from 1,8-octanediol and L-valine, (1-PHE-6) formed from 1,6-hexanediol and L-phenylalanine, and (1-PHE-8) formed from 1,8-octanediol and L-phenylalanine. 1-PHE-6 and 1-PHE-8 can be differentiated from the integration of the methylene peak at 1.06-1.14 ppm. 1-VAL-8 synthesis was confirmed based on the characteristic L-valine methyl peak at 0.96 ppm.

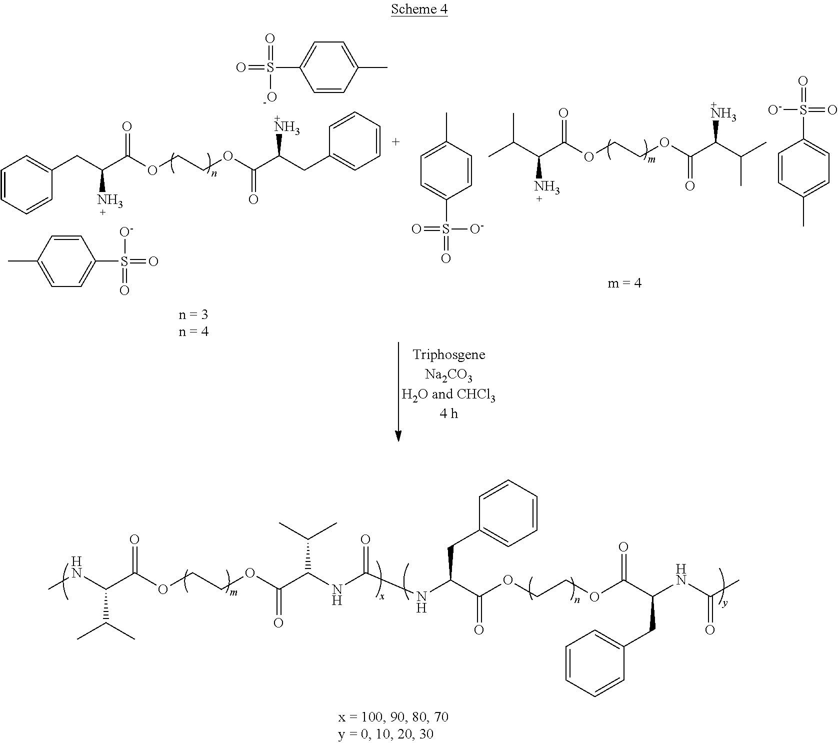

Scheme 4 shows the general synthetic scheme for forming various PEU copolymers from 1-VAL-8 and 1-PHE-6 or 1-PHE-8 monomers. In total six copolymers were synthesized with three combining 1-VAL-8 and 1-PHE-6 to form 10% PHE6 P(1-VAL8), 20% PHE6 P(1-VAL-8), and 30% PHE6 P(1-VAL-8) and three combining 1-VAL-8 and 1-PHE-8 to form 10% PHE8 P(1-VAL-8), 20% PHE8 P(1-VAL-8), and 30% PHE8 P(1-VAL-8). All polymers were synthesized utilizing triphosgene as a coupling agent (PEU forming compound) to couple monomers and form urea moieties.

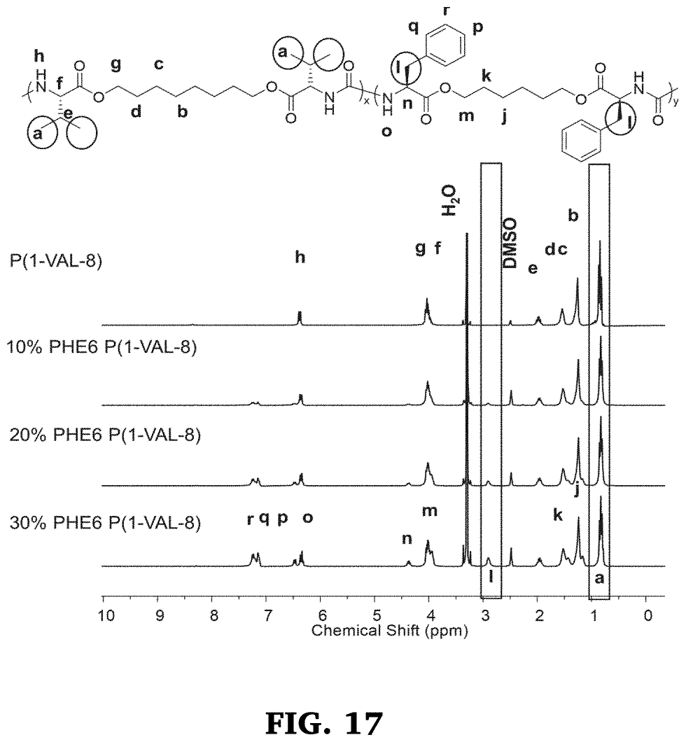

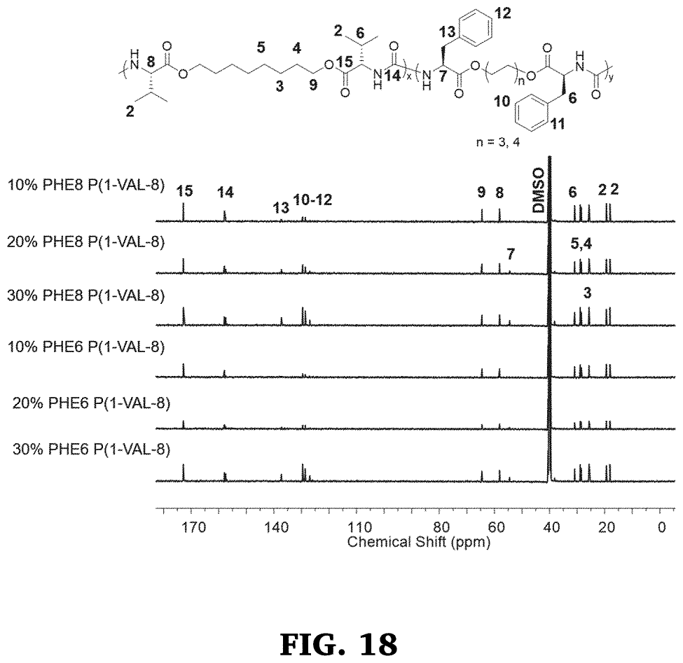

In these experiments, poly(ester urea)s were synthesized by combining 1-VAL-8 with one of the two L-phenylalanine monomers in varying mole ratios of 90:10, 80:20, and 70:30 respectively with triphosgene through interfacial polymerization based on previously published work to produce the 10% PHE6 P(1-VAL8), 20% PHE6 P(1-VAL-8), 30% PHE6 P(1-VAL-8), 10% PHE8 P(1-VAL-8), 20% PHE8 P(1-VAL-8), and 30% PHE8 P(1-VAL-8) polymers tested. See Scheme 4, above; see also, Yu, J.; Lin, F.; Lin, P.; Gao, Y.; Becker, M. L. “Phenylalanine-based poly(ester urea): Synthesis, characterization, and in vitro degradation.” Macromolecules 2014 DOI: 10.1021/ma401752b and Yu, J.; Lin, F.; Becker, M. L. “Branched amino acid based poly(ester urea)s with tunable thermal and water uptake properties.” Macromolecules 2015 DOI: 10.1021/acs.macromol.5b00376, the disclosures of which are incorporated herein by reference. The 1-PHE-6 or 1-PHE-8 monomers were chosen for their hydrophobic nature when compared to 1-VAL-8, as it was predicted that this hydrophobicity would slow in vivo degradation, when compared to the L-valine PEUs described above. Polymer synthesis was confirmed through ¹H-NMR (FIGS. 16, 17) and ¹³C-NMR (FIG. 18)). The copolymer spectra (30% PHE6 P(1-VAL-8), 20% PHE6 P(1-VAL-8), 10% PHE6 P(1-VAL-8), 30% PHE8 P(1-VAL-8), 20% PHE8 P(1-VAL-8), and 10% PHE8 P(1-VAL-8) were compared to P(1-VAL-8). Monomer incorporation was confirmed by comparing the twelve methyl L-valine protons denoted “a” with the four L-phenylalanine methylene protons denoted “1”. (See FIGS. 16, 17) All yields were obtained post-precipitation. Molecular mass values (Mₙ and M_{w}) were calculated from a linear polystyrene standard (Table 5). Molar mass distribution (Dm) for PEU copolymers were below the theoretical value of 2 because low molecular weight chains were fractioned during precipitation.

TABLE 5
Physical properties

| | T_{g-a}* | T_{g-b}* | T_{d}^{†} | Mₙ^{‡} | M_{w}^{‡} | |
|---|---|---|---|---|---|---|
| Polymer | (° C.) | (° C.) | (° C.) | (kDa) | (kDa) | Ðₘ^{‡} |
| 10% PHE8 P(1-VAL-8) | 34 | 42 | 297 | 49 | 84 | 1.7 |
| 20% PHE8 P(1-VAL-8) | 36 | 44 | 287 | 68 | 105 | 1.5 |
| 30% PHE8 P(1-VAL-8) | 28 | 42 | 278 | 69 | 108 | 1.6 |
| 10% PHE6 P(1-VAL-8) | 28 | 53 | 268 | 44 | 60 | 1.4 |
| 20% PHE6 P(1-VAL-8) | 27 | 57 | 278 | 56 | 88 | 1.6 |
| 30% PHE6 P(1-VAL-8) | 29 | 48 | 340 | 57 | 95 | 1.7 |

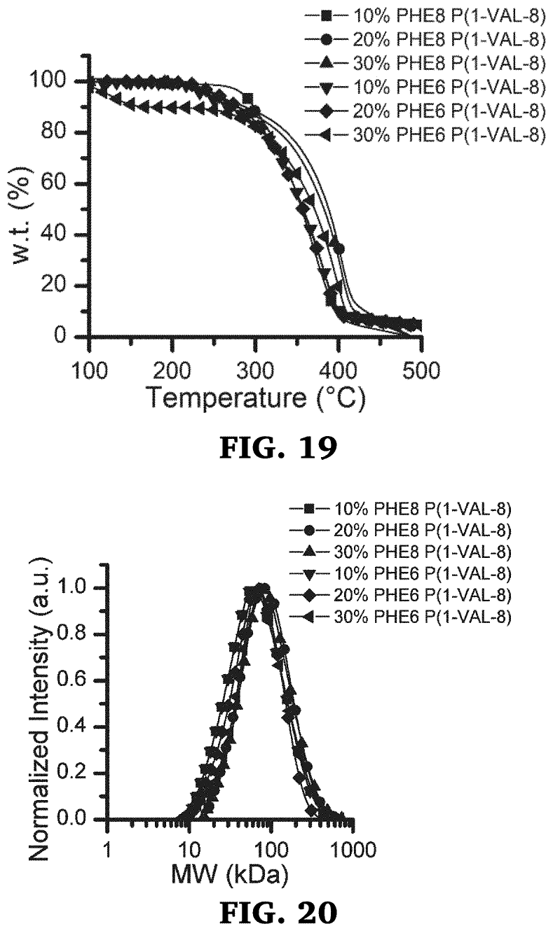

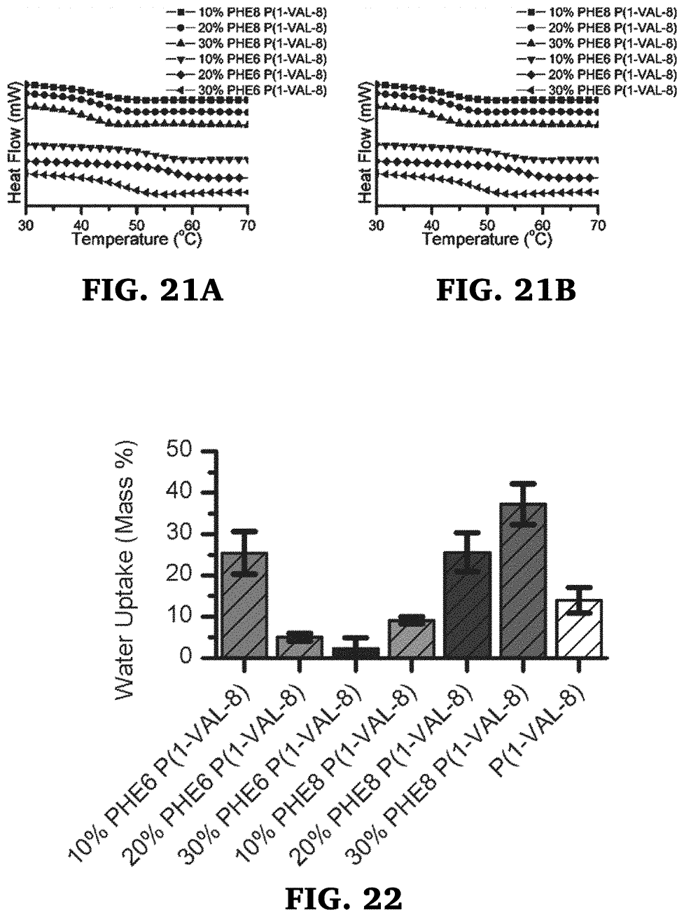

Physical Properties: Thermal properties of PEU copolymers were determined using thermogravimetric analysis (TGA) and differential scanning calorimetry (DSC) (Table 5). TGA curves (FIG. 19) show high degradation temperatures (T_{d}) which allow for these materials to be thermally processed through compression molding. This is ideal as any commercialized material will be more viable is it has thermal stability through processing and storage. Size-exclusion chromatography (SEC) was performed to determine the copolymer analogues molecular weights (FIG. 20). Reported molecular weights are post-precipitation. The molar mass distributions (Din) are less than the theoretical value of 2 because the low molecular weight fractions are lost during precipitation. DSC curves show the glass transition temperatures (T_{g}) for PEU copolymer analogues taken using TA Q10 (FIG. 21A). The T_{g} for 30% PHE6 P(1-VAL-8), 20% PHE6 P(1-VAL-8), and 10% PHE6 P(1-VAL-8) are all close within a 3° C. temperature range between 27-29° C. The T_{g} for copolymers of 30% PHE8 P(1-VAL-8), 20% PHE8 P(1-VAL8), and 10% PHE8 P(1-VAL-8) span a range of 8° C. falling between 36-28° C. All six copolymer analogues had T_{g} values within an 8° C. range and no change could be correlated based on molar feed ratio. This is attributed to the relatively low molar incorporation of 1-PHE-6 or 1-PHE-8 to P(1-VAL-8) and instrument limitations. Copolymer analogues were further characterized using the TA Q200 (FIG. 21B) and T_{g} values were recorded. PHE6 P(1-VAL-8) analogues displayed a higher T_{g} than the PHE8 P(1-VAL-8) derivatives with values falling between 48-57° C. and 42-44° C. respectively. This was expected as the PHE6 analogues have a shorter diol-chain length which leads to decreased chain mobility and subsequent elevated T_{g}. All six copolymer analogues had T_{g} values greater than the P(1-VAL-8) homopolymer. This is attributed to the L-phenylalanine side chain restricting chain mobility which elevates the T_{g}.

Water Uptake:

Water uptake studies were conducted to assess hydrolytic degradation of PEU copolymers. (See Example 17, below). Previously published work has shown that the rate of water uptake correlates to PEU degradation as a degradation mechanism is through hydrolytic degradation of the ester moiety. See, Gao, Y.; Childers, E. P.; Becker, M. L. “Poly (ester urea)s for Vascular Tissue Engineering.” 2015 DOI: 10.1021/acsbiomaterials.5b00168, the disclosure of which is encorporated herein by reference. P(1-VAL-8) showed in vivo degradation that was more rapid than desired for hernia-mesh repair applications as a loss in mechanical properties was observed. The incorporation of the hydrophobic L-phenylalanine side chain is believed to slow degradation when compared to L-valine. All six PEU copolymer analogues and a P(1-VAL-8) control were studied to assess the effects 1-PHE-6 and 1-PHE-8 incorporation have on P(1-VAL-8) and how that relates to water uptake (See, FIG. 22). Of the PHE6 P(1-VAL-8) analogues, the 20% PHE6 P(1-VAL-8) and 30% PHE6 P(1-VAL-8) displayed lower water uptake (5.03±0.92 and 2.24±2.66% respectively) when compared to P(1-VAL-8) (13.99±3.14%). This was ideal as the incorporation of PHE6 monomer slows water uptake and consequent hydrolytic degradation which leads to sustained mechanical properties which is an improvement from P(1-VAL-8) for hernia-repair applications. Only 10% PHE8 P(1-VAL-8) of the PHE8 P(1-VAL-8) analogues displayed lower water uptake (9.09±0.87%). While the L-phenylalanine plays a role in water uptake, there is a second competing factor that involves L-phenylalanine disruption of the interchain packing which could allow greater void volume leading to an increase in water uptake. This was observed in the 10% PHE6 P(1-VAL-8), 20% PHE8 P(1-VAL-8), and 30% PHE8 P(1-VAL-8) (25.46±5.18, 25.61±4.63, and 37.22+4.94% respectively).

Mechanical Properties:

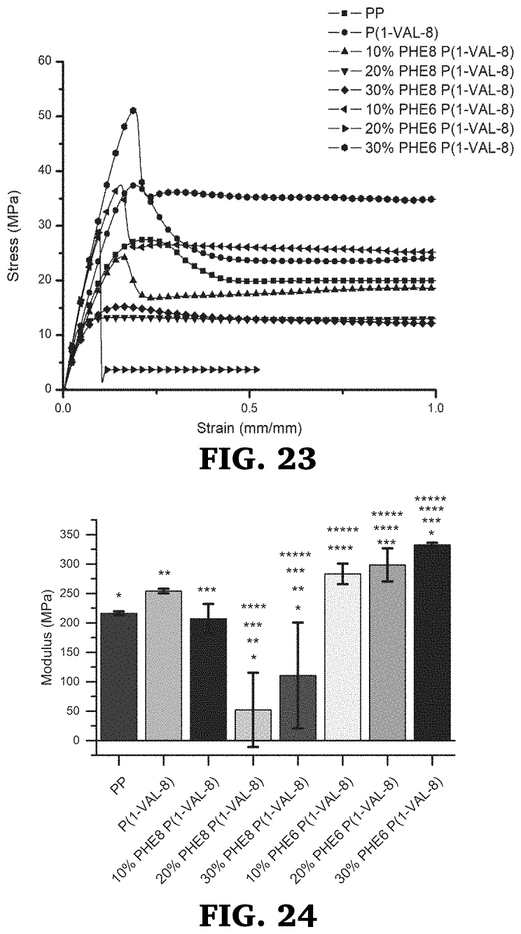

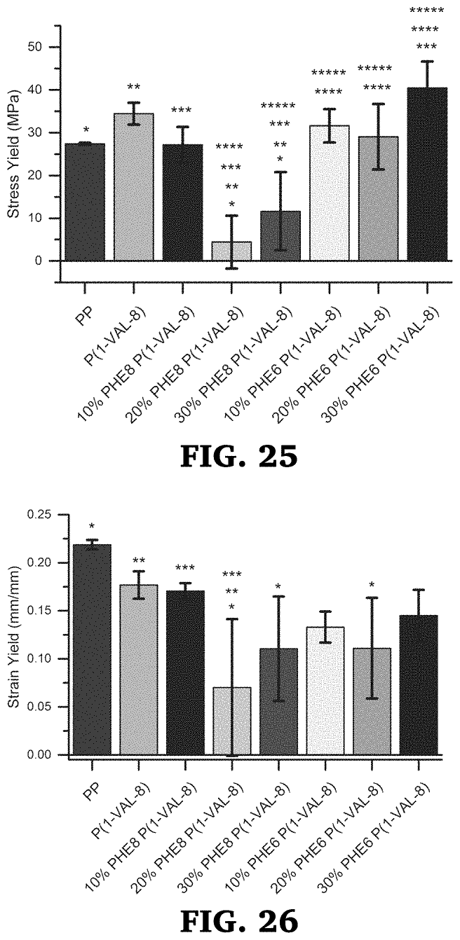

Uniaxial Tensile Testing: Tensile testing was performed on all six PEU copolymers, P(1-VAL-8), and PP with stress and strain curves (FIG. 23) being reported. (See, Example 14, below). The Young's moduli values were taken at 10% strain (FIG. 24). Stress at yield (a) (FIG. 25) and strain at yield (ε_{y}) (FIG. 26) were measured from the end of the linear elastic region and all values were reported (See, Table 6).

TABLE 6
Uniaxial mechanical properties comparison

| Polymer | | |
|---|---|---|
| PP | Modulus (MPa) | 216.1 ± 3.2 |
| | σ_{y} (MPa) | 27.4 ± 0.2 |
| | ε_{y} (mm/mm) | 0.2 |
| P(1-VAL-8) | Modulus (MPa) | 254.0 ± 3.9 |
| | σ_{y} (MPa) | 34.4 ± 2.6 |
| | ε_{y} (mm/mm) | 0.2 |
| 10% PHE8 P(1-VAL-8) | Modulus (MPa) | 207.0 ± 24.9 |
| | σ_{y} (MPa) | 27.2 ± 4.2 |
| | ε_{y} (mm/mm) | 0.2 |
| 20% PHE8 P(1-VAL-8) | Modulus (MPa) | 52.1 ± 63.0 |
| | σ_{y} (MPa) | 4.4 ± 6.2 |
| | ε_{y} (mm/mm) | 0.1 |
| 30% PHE8 P(1-VAL-8) | Modulus (MPa) | 110.6 ± 89.8 |
| | σ_{y} (MPa) | 11.6 ± 9.2 |
| | ε_{y} (mm/mm) | 0.1 |
| 10% PHE6 P(1-VAL-8) | Modulus (MPa) | 283.2 ± 17.5 |
| | σ_{y} (MPa) | 31.6 ± 3.9 |
| | ε_{y} (mm/mm) | 0.1 |
| 20% PHE6 P(1-VAL-8) | Modulus (MPa) | 298.5 ± 28.2 |
| | σ_{y} (MPa) | 29.1 ± 7.6 |
| | ε_{y} (mm/mm) | 0.1 |
| 30% PHE6 P(1-VAL-8) | Modulus (MPa) | 332.5 ± 3.5 |
| | σ_{y} (MPa) | 40.5 ± 6.2 |
| | ε_{y} (mm/mm) | 0.1 |

The Young's modulus, stress at yield (σy) and strain at yield (εy) were measured and recorded from the stress-strain curves from uniaxial tensile testing. Values reported are an average of 4-6 samples.

When comparing moduli values, incorporation of 1-PHE-6 showed a significant enhancement for 30% PHE6 P(1-VAL-8) compared to PP. Although not significant, 10% PHE6 P(1-VAL-8) and 20% PHE6 P(1-VAL-8) moduli averages were greater than that of PP and P(1-VAL-8). The increase in moduli values was ideal as degradation for these materials is expected upon implementation which will lead to a drop in mechanical properties. For degradable devices, temporarily bolstered mechanical properties can be utilized as a way to maintain the required mechanical properties throughout the lifetime of the implanted material. 10% PHE8 P(1-VAL8), 20% PHE8 P(1-VAL-8), and 30% PHE8 P(1-VAL-8) all exhibited brittle behavior which was observed along with a drop in moduli values. All PHE8 P(1-VAL-8) polymers exhibited lower moduli values than all PHE6 P(1-VAL-8) polymers with 20% PHE8 P(1-VAL-8) and 30% PHE8 P(1-VAL-8) being significantly lower than their PHE6 P(1-VAL-8) counterparts. This was an expected result as PHE8 copolymer analogues have longer diol-chain lengths than the PHE6 copolymer counterparts which leads to an increase in chain flexibility and subsequent drop in material stiffness. Similar trends were observed when comparing σ_{y} values for the PHE8 P(1-VAL-8) and PHE6 P(1-VAL-8) polymers with 20% PHE8 P(1-VAL-8) and 30% PHE8 P(1-VAL-8) being significantly lower than their PHE6 P(1-VAL-8) counterparts. While not significant, the average σ_{y} values for 10% PHE6 P(1-VAL-8) and 30% PHE6 P(1-VAL-8) were greater than PP. The ε_{y} for all PEU copolymers were all lower than PP with 20% PHE8 P(1-VAL-8), 30% PHE8 P(1-VAL-8), and 20% PHE6 P(1-VAL-8) being significant. The ε_{y} values for all PEU copolymers were not different from P(1-VAL-8) except for 20% PHE8 P(1-VAL-8) which was significantly less. Being able to stiffen PEUs through the incorporation of PHE6 or PHE8 monomers without altering the ε_{y} values was promising as staying in an elastic recoverable region relevant to physiological conditions is important to device success.

Burst-Test Mechanics Composite Films:

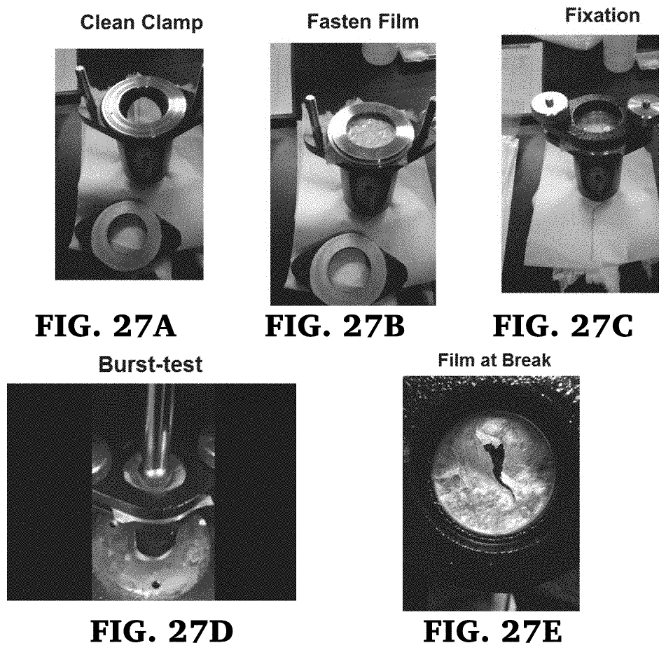

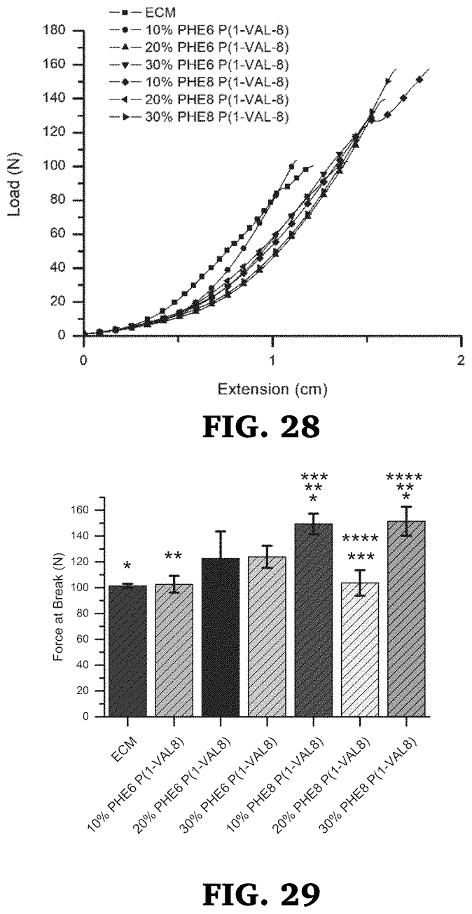

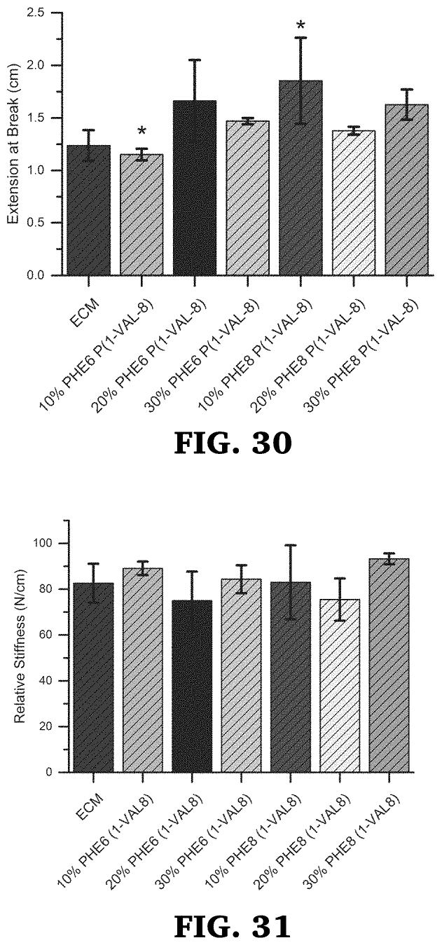

Extra cellular matrix (ECM) is a currently employed material for treatment of hernia-mesh repair. It is heralded for its promotion of native tissue growth however ECM often suffers from rapid degradation which can lead to mechanical property loss and recurrence. To alleviate this problem, PEU copolymers were blade coated on top of ECM in hopes of enhancing the mechanical properties. (See, Example 15, below). Samples were subjected to burst testing (FIGS. 27A-E) and the force versus extension curves were recorded (FIG. 28) with force at break and extension at break being recorded (See, Table 7; FIGS. 29, 30).

TABLE 7
Burst-test PEU-ECM mechanical properties comparison

Polymer

| | | |
|---|---|---|
| ECM | Relative Stiffness (N/cm) | 82.4 ± 8.6 |
| | Force at Break (N) | 101.3 ± 1.6 |
| | Extension at Break (cm) | 1.2 ± 0.1 |
| 10% PHE8 P(1-VAL-8)-ECM | Relative Stiffness (N/cm) | 83.0 ± 16.2 |
| | Force at Break (N) | 149.4 ± 7.9 |
| | Extension at Break (cm) | 1.9 ± 0.4 |
| 20% PHE8 P(1-VAL-8)-ECM | Relative Stiffness (N/cm) | 75.5 ± 9.2 |
| | Force at Break (N) | 103.7 ± 9.9 |
| | Extension at Break (cm) | 1.4 ± <0.1 |
| 30% PHE8 P(1-VAL-8)-ECM | Relative Stiffness (N/cm) | 93.2 ± 2.4 |
| | Force at Break (N) | 151.4 ± 11.3 |
| | Extension at Break (cm) | 1.6 ± 0.1 |
| 10% PHE6 P(1-VAL-8)-ECM | Relative Stiffness (N/cm) | 89.1 ± 3.0 |
| | Force at Break (N) | 102.6 ± 6.5 |
| | Extension at Break (cm) | 1.2 ± 0.1 |
| 20% PHE6 P(1-VAL-8)-ECM | Relative Stiffness (N/cm) | 75.0 ± 12.7 |
| | Force at Break (N) | 122.5 ± 30.0 |
| | Extension at Break (cm) | 1.7 ± 0.4 |
| 30% PHE6 P(1-VAL-8)-ECM | Relative Stiffness (N/cm) | 84.4 ± 6.1 |
| | Force at Break (N) | 123.9 ± 8.5 |
| | Extension at Break (cm) | 1.5 ± <0.1 |

Effective stiffness (FIG. 31) was determined by dividing the force by extension at break for each sample. There was no increased stiffness when ECM was coated with PEU copolymers. This was considered ideal as mesh stiffness can correlate with patient discomfort. The extension at break (FIG. 30) was recorded and although not significant, all PEU-ECM composites had equal or greater extension compared to ECM. If a hernia implant fractures or breaks then the device is rendered less effective which is a major reason for a device material to possess elastic properties with extension lengths relevant to physiological conditions. PEU-ECM composite analogues possess this enhanced elastic property without increased stiffness which is a significant upgrade from standalone ECM as these materials can extend to further lengths than ECM without failure. Another significant upgrade was the amount of force required to rupture the composite films. The force at break (FIG. 29) was greater for all PEU-ECM copolymer analogues compared to ECM with 10% PHE8 P(1-VAL-8) and 30% PHE8 P(1-VAL8) being significantly greater. Blade coating PEU copolymer films on ECM successfully enhance mechanical properties of ECM without increasing the effective stiffness which makes these composite films a viable candidate for hernia-mesh repair.

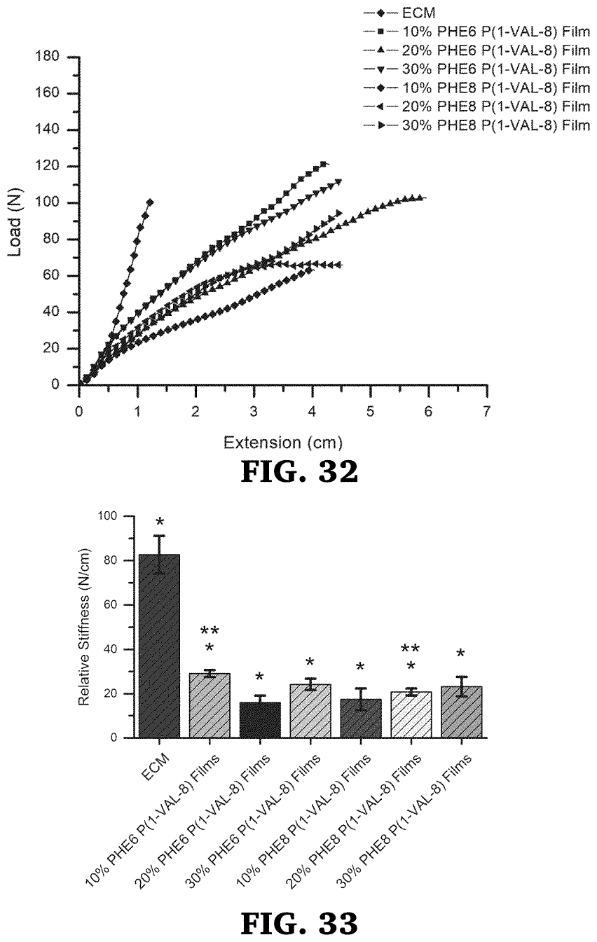

Burst-Test Mechanics Free-Standing Films. While enhancing ECM films is an attractive option, creating a new stand-alone film that fulfills hernia-mesh repair requirements would be more attractive as it would reduce the demand for ECM which is precluded by manufacturing cost. Burst testing was performed as previously described and the force versus extension curves are shown (FIG. 32) with effective stiffness, force at break, and extension at break being reported. (See, Table 8; FIGS. 33-35). (See also, Example 16, below).

TABLE 8
Free-standing film burst-test mechanical properties

Polymer

| | | |
|---|---|---|
| ECM | Relative Stiffness (N/cm) | 82.6 ± 8.6 |
| | Force at Break (N) | 101.3 ± 1.6 |
| | Extension at Break (cm) | 1.2 ± 0.1 |
| 10% PHE8 P(1-VAL-8)-ECM | Relative Stiffness (N/cm) | 17..5 ± 4.9 |
| | Force at Break (N) | 54.2 ± 9.8 |
| | Extension at Break (cm) | 3.2 ± 0.8 |
| 20% PHE8 P(1-VAL-8)-ECM | Relative Stiffness (N/cm) | 20.8 ± 1.6 |
| | Force at Break (N) | 64.1 ± 27.0 |
| | Extension at Break (cm) | 3.1 ± 1.4 |
| 30% PHE8 P(1-VAL-8)-ECM | Relative Stiffness (N/cm) | 23.2 ± 4.4 |
| | Force at Break (N) | 88.1 ± 6.4 |
| | Extension at Break (cm) | 3.9 ± 0.7 |
| 10% PHE6 P(1-VAL-8)-ECM | Relative Stiffness (N/cm) | 29.1 ± 1.6 |
| | Force at Break (N) | 118.0 ± 25.3 |
| | Extension at Break (cm) | 4.0 ± 0.7 |
| 20% PHE6 P(1-VAL-8)-ECM | Relative Stiffness (N/cm) | 16.1 ± 3.1 |
| | Force at Break (N) | 90.9 ± 10.6 |
| | Extension at Break (cm) | 5.8 ± 1.0 |
| 30% PHE6 P(1-VAL-8)-ECM | Relative Stiffness (N/cm) | 24.2 ± 2.5 |
| | Force at Break (N) | 110.3 ± 5.9 |
| | Extension at Break (cm) | 4.6 ± 0.7 |

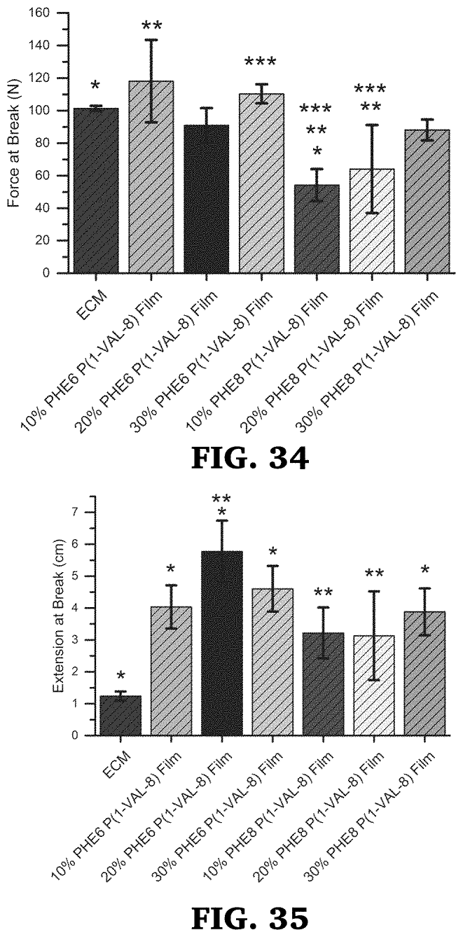

Effective stiffness (FIG. 33) for these films was decreased when compared to ECM which could make these films feel more comfortable to the patient as the material may now feel closer to that of soft-tissue. The force at break for the PEU free-standing films (FIG. 34) did not change significantly from ECM however, the extension at break (FIG. 35) for PEU free-standing films was far superior to that of ECM. This is thought to be ideal as improved extension will prevent device failure.

Current biologic hernia-mesh repair materials leave much to be desired in sustained mechanical support. Thus as series of L-valine-co-L-phenylalanine poly(ester urea) copolymers were synthesized and their mechanical properties were assessed to determine their use in composite films with ECM or as free-standing hernia-mesh films. These polymers are ideal as they are easily processable through compression molding or flow coating because of high degradation temperatures and solubility in relatively benign solvents. PEU-ECM composite films displayed enhanced mechanical properties compared to stand alone ECM films which are prone to mechanical failure from implant degradation. Free-standing PEU copolymer films also elicited a significant increase in extension at break compared to ECM. These L-valine-co-L-phenylalanine poly(ester urea) copolymers are viable alternative materials for use in hernia mesh applications with enhanced mechanical properties compared to ECM.

### EXAMPLES

The following experiments are offered to more fully illustrate the invention, but are not to be construed as limiting the scope thereof. Further, while some of examples may include conclusions about the way the invention may function, the inventor do not intend to be bound by those conclusions, but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Materials

1,8-octanediol, 1,6-hexanediol, 1,10-decanediol, 1,12-dodecanediol, triphosgene, sodium carbonate, 1,1,1-tris(hydroxymethyl)ethane, and p-toluenesulfonic acid monohydrate were purchased from Sigma Aldrich (Milwaukee, Wis.). Toluene, chloroform, acetone, and N,N-dimethylformamide were purchased from Fisher Scientific (Pittsburgh, Pa.). Boc-o-benzyl tyrosine and L-phenylalanine were purchased from Acros (Pittsburgh, Pa.) and L-valine was purchased from Bachem (Torrance, Calif.) (Examples 1-9) and Acros (Pittsburgh, Pa.) (Examples 10-17). SIS-ECM was provided by Cook Medical and used as provided. All solvents were reagent grade and all chemicals were used without further purification unless otherwise stated.

### Characterization

¹H NMR ad ¹³C NMR spectra were conducted using a 300 MHz and 500 MHz Varian NMR spectrophotometer respectively. Chemical shifts are reported in ppm (δ) and referenced to residual solvent resonances (¹H NMR DMSO-d₆ 2.50 ppm). Multiplicities were explained using the following abbreviations: s=singlet, d=doublet, t=triplet, br=broad singlet, and m=multiplet. Size exclusion chromatography (SEC) was performed using an EcoSEC HLC-8320GPC (Tosoh Bioscience, LLC) equipped with a TSKgel SuperH-RC 6.0 mml.D.×15 cm mixed bed column and refractive index (RI) detector. The number average molecular mass (Mₙ), weight average molecular mass (M_{w}), and molecular mass distribution (Ð_{M}) for each sample was calculated using a calibration curve determined from polystyrene standards (PStQuick MP-M standards, Tosoh Bioscience LLC) with DMF (with 0.01 M LiBr) as eluent flowing 1.0 mL/min at 50° C. For Examples 1-9, differential scanning calorimetry (DSC) was performed using a TA Q2000 with heating and cooling cycle ramps of 10° C./min in the temperature range of 0-100° C. For Examples 10-17, differential scanning calorimetry (DSC) was performed using multiple instruments including a TA Q200 and a TA Q10 with heating and cooling cycles (20° C./min) with temperature sweeps from 0-100° C. The glass transition temperature (T_{g}) was determined from the midpoint of the second heating cycle endotherm. Thermogravimetric analysis (TGA) was performed using a TA Q500 with heating ramps of 20° C./min in the temperature range from 0-500° C./min. The degradation temperature (T_{d}) was determined from 10% mass loss. Surface topology images were obtained from scanning electron microscopy (SEM). Using a JEOL USA SEM, samples were sputter-coated with gold and scanned with 2.0 kV excitation at 750× magnification. Statistical analysis was performed using a Tukey post-hoc ANOVA. Sample population normality and homogeneity were not considered because of limited sample size.

### Example 1

### Synthesis of Di-p-toluenesulfonic Acid Salts of Bis(L-valine)-Octane 1, 8-Diester Monomer (1-VAL-8)

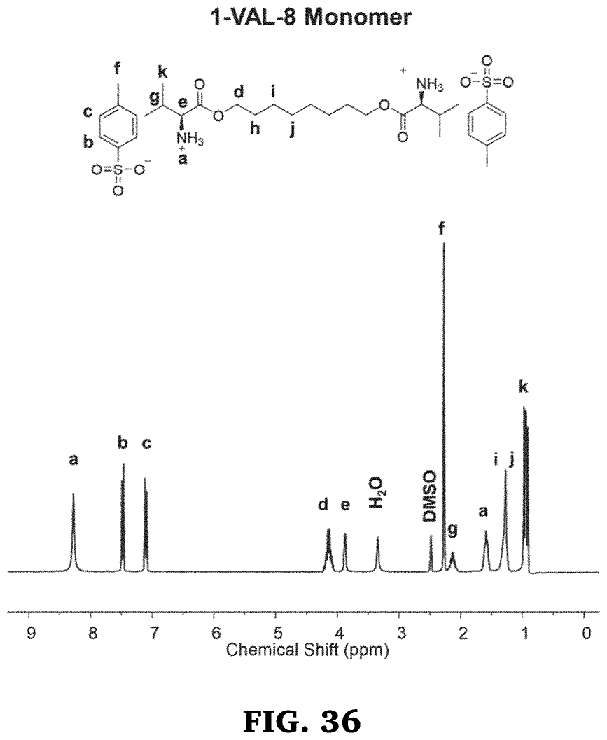

Synthesis of di-p-toluenesulfonic acid salts of bis(L-valine)-octane 1,8-diester (1-VAL-8) was carried out following previously published procedures. See, Yu, J.; Lin, F.; Lin, P.; Gao, Y.; Becker, M. L. “Phenylalanine-based poly(ester urea): Synthesis, characterization, and in vitro degradation.” Macromolecules 2014 DOI: 10.1021/ma401752b, the disclosure of which is incorporated herein by reference. Briefly, 1,8-octanediol (43.8 g, 0.3 mol, 1 eq.), L-valine (73.8 g, 0.63 mol, 2.3 eq.), p-toluenesulfonic acid monohydrate (131.3 g, 0.69 mol, 2.4 eq.), and toluene (1300 mL) were added to a 3 L 3-neck round bottom flask and mixed with overhead mechanical stirring. A Dean-Stark Trap was attached to the round bottom flask and the reaction was heated to reflux for 24 h. The reaction was cooled to ambient temperature, and the resulting white precipitate was isolated by vacuum filtration using a Buchner funnel. The product was recrystallized by dissolving in boiling water (2 L), vacuum filtering hot, and cooling to room temperature to afford a white solid precipitate. The precipitate was collected via filtration and the recrystallization process was performed three times for purity (166 g, 79% yield). ¹H NMR (300 MHz, DMSO-d₆): δ=0.95-0.99 (m, 12H, —CH(CH₃)₂), 1.24-1.35 (s, 8H, —COOCH₂CH₂(CH₂)₄—), 1.55-1.65 (m, 4H, —COOCH₂CH₂(CH₂)₄CH₂—), 2.06-2.22 (m, 2H, (CH₃)₂CH—), 2.26-2.31 (s, 6H, —CH₃Ar—), 2.50 (m, DMSO), 3.33-3.38 (s, H₂O), 3.88-3.90 (d, J=4.3 Hz, 2H, +NH₃CHCOO—), 4.08-4.24 (m, 4H, —COOCH₂CH₂(CH₂)₄—), 7.10-7.14 (d, J=8.2 Hz, 4H, aromatic H), 7.48-7.50 (d, J=8.1 Hz, 4H, aromatic H), 8.25-8.33 (br, 6H, —NH₃⁺). (See, FIGS. 1, 36)

### Example 2

### Synthesis of Di-p-toluenesulfonic Acid Salts of Bis(L-valine)-Decane 1,10-Diester Monomer. (1-VAL-10)

Synthesis of di-p-toluenesulfonic acid salts of bis(L-valine)-decane 1,10-diester (1-VAL-10) was carried out using the method described I Example 1, above except that 1,10-decanediol was used in place of 1,8-octanediol (154 g, 71% yield). ¹H NMR (300 MHz, DMSO-d₆): δ=0.93-1.00 (m, 12H, —CH(CH₃)₂—), 1.22-1.33 (s, 12H, —COOCH₂CH₂(CH₂)₆—), 1.55-1.64 (m, 4H, —COOCH₂CH₂(CH₂)₄CH₂—), 2.09-2.21 (m, 2H, (CH₃)₂CH—), 2.28-2.31 (s, 6H, —CH₃Ar—), 2.50 (m, DMSO), 3.30-3.35 (s, H₂O), 3.87-3.91 (d, J=4.5 Hz, 2H, +NH₃CHCOO—), 4.08-4.24 (m, 4H, —COOCH₂CH₂(CH₂)₆—), 7.10-7.13 (d, J=7.8 Hz, 4H, aromatic H), 7.47-7.51 (d, J=7.8 Hz, 4H, aromatic H), 8.27-8.31 (br, 6H, —NH₃⁺). (See, FIG. 1)

### Example 3

### Synthesis of Di-p-toluenesulfonic Acid Salts of Bis-(l-valine)-Dodecane 1,12-Diester Monomer. (1-VAL-12)

Synthesis of di-p-toluenesulfonic acid salts of bis(L-valine)-dodecane 1,12-diester (1-VAL-12) was carried out using the method described above (106 g, 82% yield). ¹H NMR (300 MHz, DMSO-d₆): δ=0.90-0.98 (m, 12H, —CH(CH₃)₂), 1.22-1.27 (s, 16H, —COOCH₂CH₂(CH₂)₈—), 1.53-1.63 (m, 4H, —COOCH₂CH₂(CH₂)₈CH₂—), 2.07-2.18 (m, 2H, (CH₃)₂CH⁺—), 2.27-2.29 (s, 6H, —CH₃Ar—), 2.50 (m, DMSO), 3.29-3.33 (s, H₂O), 3.87-3.90 (d, J=4.3 Hz, 2H, +NH₃CHCOO—), 4.06-4.22 (m, 4H, —COOCH₂CH₂(CH₂)₈—), 7.08-7.11 (d, J=7.9 Hz, 4H, aromatic H), 7.45-7.49 (d, J=8.1 Hz, 4H, aromatic H), 8.25-8.28 (br, 6H, —NH₃⁺). (See, FIG. 1)

### Example 4

### Synthesis of Hydrochloric Acid Salts of Tri-O-benzyl-L-tyrosine-1, 1, 1-trimethylethane Triester Monomer. (Triol-TYR)

Synthesis of hydrochloric acid salts of Tri-O-benzyl-L-tyrosine-1,1,1-trimethylethane triester monomer (Triol-TYR) was carried out following previously published procedures. See, Yu, J.; Lin, F.; Becker, M. L. Branched amino acid based poly(ester urea)s with tunable thermal and water uptake properties. Macromolecules 2015 DOI: 10.1021/acs.macromol.5b00376, the disclosure of which is incorporated herein by reference. The branched monomer was formed through the esterification between 1,1,1-tri(hydroxylmethyl)ethane and Boc-O-benzyl-L-tyrosine. In a 500 mL RBF, 1,1,1-tri(hydroxylmethyl)ethane (2.00 g, 16 mmol, 1.0 eq.), Boc-O-benzyl-L-tyrosine (22.20 g, 60 mmol, 3.75 eq.), and 4-(N,N-dimethylamino)puridinium 4-toluenesulfonate (DPTS, 3.00 g, 10 mmol, 0.6 eq.) were dissolved in a minimum amount of DMF. Once dissolved, the reaction was placed in an ice bath for 10 minutes followed by syringe addition of 1,3-diisopropyl carbodiimide (DIC, 10.14 mL, 80 mmol, 5 eq.). The reaction was allowed to gradually come to ambient temperature while stirring for 24 h, and a yellow precipitate formed. DMF was removed under reduced pressure using a vacuum transfer and the remaining solid was dissolved in a minimal amount of chloroform. The solution was washed (3×) with sodium bicarbonate and the organic solution was concentrated for column chromatography purification. Silica gel was used as the stationary phase with hexane/ethyl acetate (4:1 v/v) mobile phase and all fractions were collected for rotary evaporation. The solvent was removed by evaporation and a yellow solid was obtained (12.2 g, 73%). ¹H NMR (300 MHz, DMSO-d₆): δ=0.81-0.87 (s, 3H, —CCH₃), 1.26-1.30 (s, 27H, CH₃ in Boc protecting group), 2.50 (m, DMSO), 2.72-2.94 (m, 6H, —CHCH₂—Ar—), 3.92-3.94 (m, 6H, —COOCH₂C—), 4.08-4.16 (m, 3H, —⁺NH₃CHCOO—), 5.00-5.03 (s, 6H, —Ar—OCH₂—Ar—), 6.86-7.42 (m, 27H, aromatic H). The boc-protected yellow solid was dissolved in HCl/dioxane (4 M) and allowed to stir under nitrogen for 3 h. The yellow solid was freeze-dried to remove solvent (11.5 g, 69%). ¹H NMR (300 MHz, DMSO-d₆): δ=0.65-0.67 (s, 3H, —CCH₃), 2.50 (m, DMSO), 2.98-3.18 (m, 6H, —CHCH₂—Ar—), 3.81-3.96 (m, 6H, —COOCH₂C—), 4.16-4.22 (m, 3H, —⁺NH₃CHCOO—), 5.02-5.04 (s, 6H, —Ar—OCH₂—Ar—), 6.91-7.42 (m, 27H, aromatic H), 8.72-8.78 (br s, 9H, +NH₃-). (See, FIG. 2)

### Example 5

### Synthesis of Linear Poly(Ester Urea)s

The syntheses of linear poly(ester urea)s were based on previously published procedures. See, Yu, J.; Lin, F.; Lin, P.; Gao, Y.; Becker, M. L. “Phenylalanine-based poly(ester urea): Synthesis, characterization, and in vitro degradation.” Macromolecules 2014 DOI: 10.1021/ma401752b and Yu, J.; Lin, F.; Becker, M. L. Branched amino acid based poly(ester urea)s with tunable thermal and water uptake properties. Macromolecules 2015 DOI: 10.1021/acs.macromol.5b00376, the disclosures of which are incorporated herein by reference. In short, interfacial polymerization of di-p-toluenesulfonic acid salts of bis(L-valine) monomers 1-VAL8, 1-VAL-10, and 1-VAL-12 was performed by dissolving the desired monomer and sodium carbonate anhydrate in distilled water (0.1 M) in a 3 L 3-neck round-bottom flask. The solution was placed in a 40° C. water bath overhead mechanical stirring until clear. Ice was added to the water bath until the temperature was cooled to 0° C. In a separate container, additional sodium carbonate (1.05 eq.) was dissolved in distilled water and added to the reaction flask and the solution was allowed to stir until clear. Separately, triphosgene (0.35 eq.) was dissolved in distilled chloroform (0.6 M) and subsequently added to the reaction flask using an addition funnel. The solution turned white immediately and was allowed to stir for 30 minutes. An additional aliquot of triphosgene (0.08 eq.) dissolved in distilled chloroform (0.6 M) was added to solution dropwise (˜1 drop/second) using the addition funnel. The reaction was stirred for 3 hours and then transferred to a separatory funnel and washed with water (3×). The organic phase was collected and precipitated in hot water to remove impurities. The product was cooled and dried under reduced pressure. The white polymer was thus collected (82-92% yield).

Poly(1-VAL-8). ¹H NMR (300 MHz, DMSO-d₆): δ=0.77-0.89 (m, 12H, —CH(CH₃)₂), 1.24-1.33 (s, 8H, —COOCH₂CH₂(CH₂)₄—), 1.50-1.58 (m, 4H, —COOCH₂CH₂(CH₂)₄CH₂—), 1.94-2.04 (m, 2H, —(CH₃)₂CHCHNH₃⁺—), 2.50 (m, DMSO), 3.29-3.33 (s, H₂O), 3.94-4.10 (m, 6H, —CHCOOCH₂CH₂(CH₂)₄—), 6.37-6.41 (s, 2H, —NH—). (M_{w}=71 kDa, Mₙ₌42 kDa, Dm=1.7, T_{g}=42° C., T_{d}=310° C.). (See, FIG. 3)

Poly(1-VAL-10). ¹H NMR (300 MHz, DMSO-d₆): δ=0.78-0.90 (s, 12H, —CH(CH₃)₂), 1.20-1.29 (s, 12H, —COOCH₂CH₂(CH₂)₆—), 1.49-1.56 (m, 4H, —COOCH₂CH₂(CH₂)₆CH₂—), 1.91-2.00 (m, 2H, (CH₃)₂CH—), 2.50 (m, DMSO), 3.30-3.34 (s, H₂O), 3.97-4.11 (m, 6H, —CHCOOCH₂CH₂(CH₂)₆—), 6.32-6.42 (s, 2H, —NH—). (M_{w}=71 kDa, Mₙ=46 kDa, Dm=1.6, T_{g}=34° C., T_{d}=339° C.). (See, FIG. 3)

Poly(1-VAL-12). ¹H NMR (300 MHz, DMSO-d₆): δ=0.81-0.87 (s, 12H, —CH(CH₃)₂), 1.21-1.27 (s, 17H, —COOCH₂CH₂(CH₂)₈—), 1.50-1.56 (m, 4H, —COOCH₂CH₂(CH₂)₈CH₂—), 1.92-2.05 (m, 2H, (CH₃)₂CH—), 2.50 (m, DMSO), 3.28-3.31 (s, H₂O), 3.95-4.11 (m, 6H, —CHCOOCH₂CH₂(CH₂)₈—), 6.32-6.42 (s, 2H, —NH—). (M_{w}=75 kDa, Mₙ=51 kDa, Dm=1.4, T_{g}=29° C., T_{d}=205° C.). (See, FIG. 3)

### Example 6

### Synthesis of Branched Poly(Ester Urea)s

The syntheses of the branched poly(ester urea)s were based on previously published procedures. See, Yu, J.; Lin, F.; Lin, P.; Gao, Y.; Becker, M. L. “Phenylalanine-based poly(ester urea): Synthesis, characterization, and in vitro degradation.” Macromolecules 2014 DOI: 10.1021/ma401752b and Yu, J.; Lin, F.; Becker, M. L. Branched amino acid based poly(ester urea)s with tunable thermal and water uptake properties. Macromolecules 2015 DOI: 10.1021/acs.macromol.5b00376, the disclosures of which are incorporated herein by reference. In short, interfacial polymerization was performed by dissolving the di-p-toluenesulfonic acid salt of bis(L-valine) monomers 1-VAL-8 or 1-VAL-10 with the hydrochloric acid salt of Triol-TYR in a molar ratio of 98:2 respectively (1.0 eq. in total), as well as sodium carbonate anhydrate (2.1 eq.) in distilled water (0.1 M) in a 3 L 3-neck round bottom flask. The solution was placed in a 40° C. water bath with overhead mechanical stirring until clear. Ice was added to the water bath until the temperature reached 0° C. Separately, additional sodium carbonate (1.05 eq.) was dissolved in distilled water and the solution was added to the reaction flask and stirred until clear. Triphosgene (0.35 eq.) was dissolved in distilled chloroform (0.6 M) and subsequently added to the reaction flask using an addition funnel. The solution turned white immediately and was allowed to stir for 30 minutes. An additional aliquot of triphosgene (0.08 eq.) dissolved in distilled chloroform (0.6 M) was added to solution dropwise (˜1 drop/second) through the addition funnel and was allowed to stir for 3 hours before transferring to a separatory funnel. The reaction mixture was washed with water (3×) and the organic phase was collected, then recrystallized in hot water. The product was allowed to cool, filtered, and dried under reduced pressure. The white polymer was thus collected (79-88% yield).

Branched PEU-2% (Bis(L-valine)-Octane 1,8-Diester Monomer and Tri-O-benzyl-L-tyrosine-1,1, 1-trimethylethane Triester Monomer with a Molar Ratio of 98:2). ¹H NMR (300 MHz, DMSO-d₆): δ=0.80-0.90 (m, 12H—CH(CH₃)₂), 1.22-1.34 (s, 8H, —COOCH₂CH₂(CH₂)₄—), 1.52-1.58 (m, 4H, —COOCH₂CH₂(CH₂)₄CH₂—), 1.95-2.02 (m, 2H, (CH₃)₂CH—), 2.50 (m, DMSO), 3.33-3.38 (s, Dioxane), 3.98-4.08 (m, 6H, —CHCOOCH₂CH₂(CH₂)₄—), 5.00-5.02 (s, —Ar—OCH₂—Ar—), 6.37-6.42 (d, J=8.9 Hz, 2H, —NH—), 6.88-7.42 (aromatic H, branched monomer), 8.25-8.33 (s, —NH—, branched monomer). (M_{w}=410 kDa, Mₙ=126 kDa, Ðₘ=3.3, T_{g}=35° C., T_{d}=301° C.). (See, FIG. 4)

Branched PEU-2% (Bis(L-valine)-Decane 1, 10-Diester Monomer and Tri-O-benzyl-L-tyrosine-1,1, 1-trimethylethane Triester Monomer with a Molar Ratio of 98:2). ¹H NMR (300 MHz, DMSO-d₆): δ=0.78-0.90 (m, 12H, —CH(CH₃)₂), 1.21-1.31 (s, 12H, —COOCH₂CH₂(CH₂)₆—), 1.51-1.58 (m, 4H, —COOCH₂CH₂(CH₂)₆CH₂—), 1.93-2.04 (m, 2H, (CH₃)₂CH—), 2.50 (m, DMSO), 3.29-3.41 (s, Dioxane), 3.97-4.08 (m, 6H, —CHCOOCH₂CH₂(CH₂)₆—), 5.01-5.04 (s, —Ar—OCH₂—Ar—), 6.35-6.43 (d, J=8.8 Hz, 2H, —NH—), 6.87-7.45 (aromatic H, branched monomer), 8.30-8.38 (s, —NH—, branched monomer). (M_{w}=137 kDa, Mₙ=68 kDa, Dm=2.0, T_{g}=31° C., T_{d}=320° C.). (See, FIG. 5)

### Example 7

### Mechanical Property Measurements

In order to compression mold PEU films, the polymers were pulverized into a fine powder using a StrandMill Grinder. Each polymer was funneled in a mold (5 cm×5 cm×0.5 mm) and then placed in a vacuum compression instrument (TMP Technical Products Corp). The polymers were melted (163° C.) and allowed to equilibrate for 30 minutes followed by degassing cycles (1000 psi) to remove air-bubbles. The polymer molds were pressed at 69 MPa, 103 MPa, and 138 MPa. The mold was then rapidly cooled to ambient temperature to afford the respective amorphous polymer films, which were then cut into tensile bars (4.76 mm×38.1 mm×0.5 mm). Elastic moduli, yield stress (σ_{y}), and yield strain (ε_{y}) were determined using tensile tests (Instron 5543 Universal Testing Machine) at 25° C. The dimensions of each specimen were measured using calipers to ensure accurate measurement. The viscoelastic linear region was determined using linear regression with R² values >0.98. The yield stress and yield strain were subsequently measured after the linear region. Statistical analyses were performed using a one-way ANOVA with Tukey post hoc analysis. A value of p<0.05 was considered significant. (See, Table 3, above).

### Example 8

### In Vivo Implant Degradation

An animal model was developed to assess efficacy of this PEU series in vivo; primarily monitoring mechanical properties and degradation. All procedures and animal handling was in accordance with the Institutional Animal Care and Use Committee (IACUC Protocol Number 16-02-5-BRD) standards, the disclosure of which is incorporated herein by reference in its entirety. In brief, tensile bars were sterilized using ethylene oxide gas (EtO) and the loss of molecular mass was assessed using SEC. The sterilized PEU tensile bars and a poly(propylene) (PP) control (n=7 for each polymer) were then subcutaneously implanted into the back of adult female Sprague-Dawley rats (n=22). All rats received an anesthetic drug cocktail (ketamine, xylazine, acepromazine, 29.6:5.95:0.53 mg/kg respectively). Isoflurane (1.0% in 100% oxygen) was additionally administered to each rat through a nose-cone throughout the surgical procedure. A scalpel was used to create four dorsal incisions (1 cm in length) equidistance apart from the spine. Hemostats were then used to tunnel and create a subcutaneous pocket followed by polymer implantation with tweezers. The incisions were then closed with Michel clips. Survival rate was 100% (22/22) for all time points (2 and 3 month). (See, FIGS. 10A-F, 11A-F, 12A-F, 13A-F, and 14A-F; Table 2, above). PEU polymer implants were collected postmortem for each time-point and subjected to further characterization.

### Example 9

### Host-Implant Interaction

Polymer samples and surrounding tissue were collected postmortem, fixed in a paraformaldehyde solution, and then embedded in paraffin wax for processing. Embedded samples were sectioned (5 μm thick) and placed on microscope slides. All slides were stained in hematoxylin and eosin (H&E) and then fixed in DPX histology mount. Slides were then taken for imaging and the fibrous capsule thickness was measured at the two and 3 month time points to assess the host-immune response. Statistical analyses were done using a one-way ANOVA with Tukey post hoc analysis. A value of p<0.01 was considered significant. (See, FIGS. 14A-F, 15A-B; Table 4)

### Example 10

### Synthesis of Di-p-toluenesulfonic Acid Salts of Bis(L-valine)-Octane 1, 8-Diester Monomer (1-VAL-8)

Synthesis of di-p-toluenesulfonic acid salts of bis(L-valine)-octane 1,8-diester (1-VAL-8) was carried out following previously published procedures. See, Yu, J.; Lin, F.; Lin, P.; Gao, Y.; Becker, M. L. Phenylalanine-based poly(ester urea): Synthesis, characterization, and in vitro degradation. Macromolecules 2014 DOI: 10.1021/ma401752b, the disclosure of which in incorporated herein by reference in its entirety. Briefly, in a 3 L 3-neck round bottom flask, 1,8-octanediol (43.8 g, 0.3 mol, 1 eq.), L-valine (73.8 g, 0.63 mol, 2.3 eq.), p-toluenesulfonic acid monohydrate (131.3 g, 0.69 mol, 2.4 eq.), and toluene (1300 mL) were added to a 1 neck flask and equipped with a stir bar. A Dean-Stark trap was fastened to the round bottom flask and the reaction was heated to 110° C. and allowed to reflux for 24 h. The reaction was cooled to room temperature, and the resulting white precipitate was isolated by vacuum filtration using a Buchner funnel. The product was dissolved in boiling water (2 L), hot vacuum filtered, and cooled to room temperature to further purify the white solid precipitate. The precipitate was collected via filtration and the recrystallization process was performed three times for purity (166 g, 79% yield). ¹H NMR (300 MHz, 303 K, DMSO-d₆): δ=0.94 (m, 12H), 1.28 (s, 8H), 1.59 (m, 4H), 2.07-2.18 (m, 2H), 2.27 (s, 6H), 2.50 (m, DMSO), 3.33-3.38 (s, H₂O), 3.89 (d, ³J_{H—H}=3.0 Hz, 2H), 4.07-4.23 (m, 4H), 7.07-7.23 (d, ³J_{H—H}=8.2 Hz, 4H, aromatic H), 7.45-7.48 (d, ³J_{H—H}=8.1 Hz, 4H, aromatic H), 8.25 (br, 6H) ppm. (See, FIG. 36)

### Example 11

### Synthesis of Di-p-toluenesulfonic Acid Salts of Bis(L-phenylalanine)-Hexane 1, 6-Diester Monomer. (1-PHE-6)

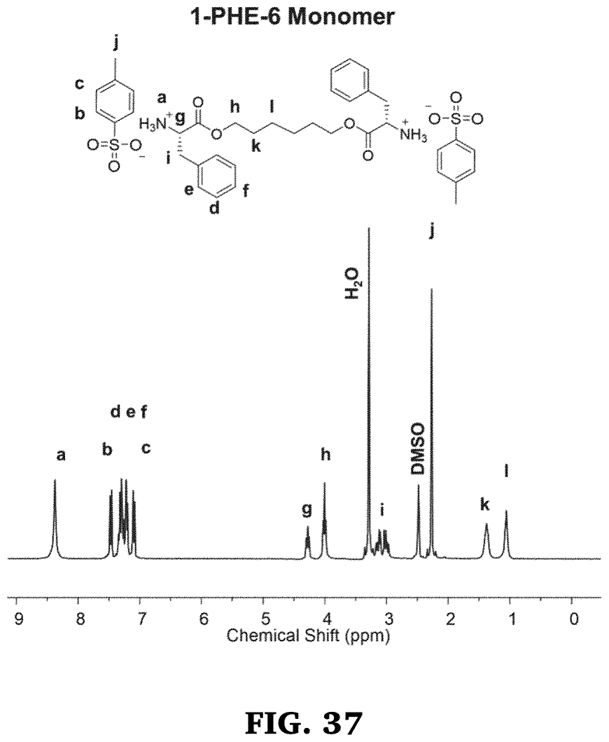

Synthesis of di-p-toluene sulfonic acid of bis(L-phenylalanine)-hexane 1,6-diester (1-PHE-6) was carried out using the method described in Example 10 above (153 g, 73% yield). ¹H NMR (300 MHz, 303 K, DMSO-d₆): δ=1.06 (s, 4H), 1.38 (m, 4H), 2.27 (s, 6H), 2.50 (m, DMSO), 2.96-3.17 (m, 4H), 4.01 (t, ³J_{H—H}=9.0 Hz, 4H), 4.28 (t, ³J_{H—H}=6.0 Hz, 2H), 7.08-7.11 (d, 4H), 7.20-7.35 (m, 10H), 7.45-7.48 (d, 4H), 8.37 (s, 6H) ppm. (See, FIG. 37)

### Example 12

### Synthesis of Di-p-toluenesulfonic Acid Salts of Bis(L-phenylalanine)-Octane 1, 8-Diester Monomer. (1-PHE-8)

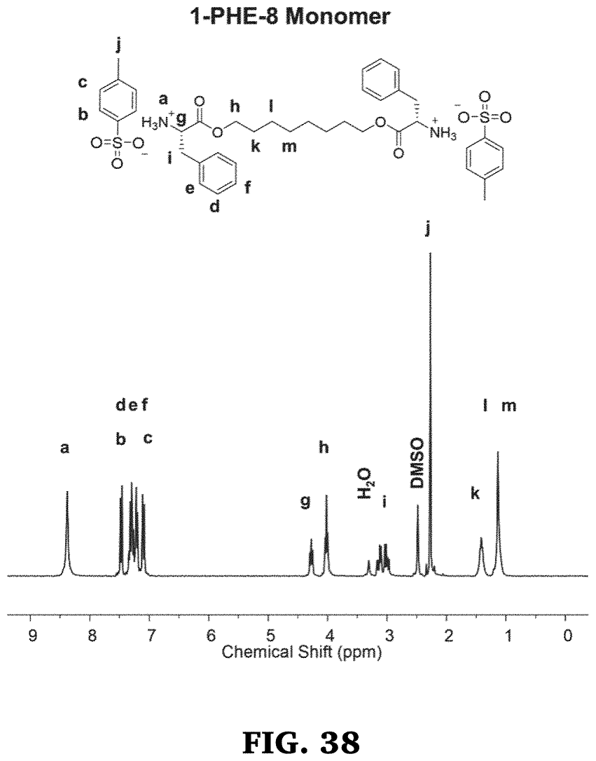

Synthesis of di-p-toluene sulfonic acid of bis(L-phenylalanine)-octane 1,8-diester (1-PHE-8) was carried out using the method described in Example 11, above (157 g, 75% yield). ¹H NMR (300 MHz, 303 K, DMSO-d₆): δ=1.14 (s, 8H) 1.41 (m, 4H), 2.27 (s, 6H), 2.50 (m, DMSO), 2.96-3.17 (m, 4H), 4.02 (t, ³J_{H—H} 6.0 Hz, 4H), 4.28 (t, ³J_{H—H}=6.0 Hz, 2H) 7.08-7.11 (d, 4H) 7.20-7.35 (m, 10H) 7.48-7.49 (d, 4H) 8.36 (s, 6H) ppm. (See, FIG. 38)

### Example 13

### Synthesis of Poly(Ester Urea) Copolymers

The synthesis was carried out according to previously published work. See, Yu, J.; Lin, F.; Lin, P.; Gao, Y.; Becker, M. L. “Phenylalanine-based poly(ester urea): Synthesis, characterization, and in vitro degradation.” Macromolecules 2014 DOI: 10.1021/ma401752b and Yu, J.; Lin, F.; Becker, M. L. “Branched amino acid based poly(ester urea)s with tunable thermal and water uptake properties.” Macromolecules 2015 DOI: 10.1021/acs.macromol.5b00376, the disclosures of which are incorporated herein by reference. Interfacial polymerization of p-toluenesulfonic acid salts of bis(L-valine) and p-toluenesulfonic acid salts of bis(L-phenylalanine) monomers 1-VAL-8, 1-PHE-6, and 1-PHE-8 was performed by dissolving the desired monomers with desired molar equivalents (1 eq. total) with sodium carbonate (3.4 eq.) in distilled water (0.1 M, 35° C.) in a 2 L 2-neck round-bottom flask. The solution was attached with an overhead mechanical stir rod and allowed to stir until clear. Triphosgene (0.35 eq.) was dissolved in distilled chloroform (0.6 M) and subsequently added to the reaction vessel through an addition funnel. The solution turned white upon addition and the solution was stirred for one hour before another aliquot of triphosgene (0.08 eq.) dissolved in distilled chloroform was added to help push the reaction to completion. After the reaction was stirred for another two hours and the product was transferred to a separatory funnel. The organic phase was then precipitated in to boiling water to remove chloroform and starting material impurities. White polymer was collected, frozen in liquid nitrogen, and then dried under reduced pressure (90-95% yield).

Poly(1-VAL-8). (P(1-VAL-8)). ¹H NMR (300 MHz, 303 K, DMSO-d₆): δ=0.77-0.89 (m, 12H, —CH(CH₃)₂), 1.24-1.33 (s, 8H, —COOCH₂CH₂(CH₂)₄—), 1.50-1.58 (m, 4H, —COOCH₂CH₂(CH₂)₄CH₂—), 1.94-2.04 (m, 2H, —(CH₃)₂CHCHNH₃⁺—), 2.50 (DMSO), 3.29-3.33 (H₂O), 3.94-4.10 (m, 6H, —CHCOOCH₂CH₂(CH₂)₄—), 6.37-6.41 (s, 2H, —NH—) ppm. (M, =71 kDa, Mₙ₌42 kDa, Dm=1.7, T_{g}=42° C., T_{d}=310° C.). (See, FIG. 3)

Poly[(1-VAL-8)₇₀-co-(1-PHE-6)₃₀]. (30% PHE6 P(1-VAL-8)). ¹H NMR (300 MHz, 303 K, DMSO-d₆): δ=0.83 (m, 12H, —CH(CH₃)₂), 1.95 (m, 2H, —NHCH(CH(CH₃)₂)C(O)O—), 2.82-2.97 (m, 4H, —NHCH(CH₂Ph)C(O)O—), 4.38 (m, 2H, —NHCH(CH₂Ph) C(O)O—), 6.36 (d, ³J_{H—H}=9 Hz, 2H, —NHCH(CH(CH₃)₂)C(O)O—), 6.48 (d, ³J_{H—H}=9 Hz, 2H, —C(O)NHC(CH₂Ph)HC(O)—), 7.13-7.28 (m, 10H, —C₆H₅), 1.18, 1.24, 1.44, 1.51, 3.95-4.05 (all remaining protons) ppm. (See, FIG. 17)¹³C NMR (500 MHz, 303 K, DMSO-d₆): δ 18.29, 19.52, 25.53, 28.72, 29.13, 30.90, 39.95, 54.68, 57.86, 64.55, 126.75, 128.45, 129.43, 137.41, 157.61, 157.84, and 172.86 ppm. (M_{w}=95 kDa, Mₙ=57 kDa, Dm=1.7, T_{g}=29° C., T_{d}=340° C.) (See, FIG. 18)

Poly[(1-VAL-8)₈₀-co-(1-PHE-6)₂₀]. (20% PHE6 P(1-VAL-8)). ¹H NMR (300 MHz, 303 K, DMSO-d₆): δ=0.83 (m, 12H, —CH(CH₃)₂), 1.95 (m, 2H, —NHCH(CH(CH₃)₂)C(O)O—), 2.82-2.97 (m, 4H, —NHCH(CH₂Ph)C(O)O—), 4.38 (m, 2H, —NHCH(CH₂Ph) C(O)O—), 6.36 (d, ³J_{H—H}=9 Hz, 2H, —NHCH(CH(CH₃)₂)C(O)O—), 6.48 (d, ³J_{H—H}=9 Hz, 2H, —C(O)NHC(CH₂Ph)HC(O)—), 7.13-7.28 (m, 10H, —C₆H₅), 1.18, 1.24, 1.44, 1.51, 3.95-4.05 (all remaining protons) ppm. (See, FIG. 17)¹³C NMR (500 MHz, 303 K, DMSO-d₆): δ 18.23, 19.49, 25.86, 28.65, 29.00, 31.01, 40.00, 54.58, 58.29, 64.64, 127.22, 128.61, 130.02, 137.86, 157.66, 158.10, 172.96 ppm. (M_{w}=88 kDa, M=56 kDa, Dm=1.6, T_{g}=27° C., T_{d}=278° C.). (See, FIG. 18)

Poly[(1-VAL-8)₉₀-CO-(1-PHE-6)jo]. (10% PHE6 P(1-VAL-8)). ¹H NMR (300 MHz, 303 K, DMSO-d₆): δ=0.83 (m, 12H, —CH(CH₃)₂), 1.95 (m, 2H, —NHCH(CH(CH₃)₂)C(O)O—), 2.82-2.97 (m, 4H, —NHCH(CH₂Ph)C(O)O—), 4.38 (m, 2H, —NHCH(CH₂Ph) C(O)O—), 6.36 (d, ³J_{H—H}=9 Hz, 2H, —NHCH(CH(CH₃)₂)C(O)O—), 6.48 (d, ³J_{H—H}=9 Hz, 2H, —C(O)NHC(CH₂Ph)HC(O)—), 7.13-7.28 (m, 10H, —C₆H₅), 1.18, 1.24, 1.44, 1.51, 3.95-4.05 (all remaining protons) ppm. (See, FIG. 17)¹³C NMR (500 MHz, 303 K, DMSO-d₆): δ 18.75, 19.81, 26.16, 29.01, 29.75, 31.44, 40.00, 55.15, 58.68, 65.08, 127.20, 129.65, 130.04, 138.13, 157.90, 158.63, and 173.12 ppm. (M_{w}=60 kDa, Mₙ=44 kDa, Ðₘ=1.4, T_{g}=28° C., T_{d}=268° C.). (See, FIG. 18)

Poly[(1-VAL-8)₇₀-co-(1-PHE-8)₃₀]. (30% PHE8 P(1-VAL-8)). ¹H NMR (300 MHz, 303 K, DMSO-d₆): δ=0.83 (m, 12H, —CH(CH₃)₂), 1.95 (m, 2H, —NHCH(CH(CH₃)₂)C(O)O—), 2.82-2.97 (m, 4H, —NHCH(CH₂Ph)C(O)O—), 4.38 (m, 2H, —NHCH(CH₂Ph) C(O)O—), 6.36 (d, ³J_{H—H}=9 Hz, 2H, —NHCH(CH(CH₃)₂)C(O)O—), 6.48 (d, ³J_{H—H}=9 Hz, 2H, —C(O)NHC(CH₂Ph)HC(O)—), 7.13-7.28 (m, 10H, —C₆H₅), 1.18, 1.24, 1.44, 1.51, 3.95-4.05 (all remaining protons) ppm. (See, FIG. 16)¹³C NMR (500 MHz, 303 K, DMSO-d₆): δ 17.76, 19.15, 25.91, 28.03, 28.66, 30.78, 40.00, 54.48, 57.96, 64.42, 126.85, 128.66, 129.43, 137.15, 156.92, 157.97, and 173.21 ppm. (M=108 kDa, Mₙ₌69 kDa, D, =1.6, T_{g}=28° C., T_{d}=278° C.). (See, FIG. 18)

Poly[(1-VAL-8)₈s-co-(1-PHE-8)₂₀]. (20% PHE8 P(1-VAL-8)). ¹H NMR (300 MHz, 303 K, DMSO-d₆): δ=0.83 (m, 12H, —CH(CH₃)₂), 1.95 (m, 2H, —NHCH(CH(CH₃)₂)C(O)O—), 2.82-2.97 (m, 4H, —NHCH(CH₂Ph)C(O)O—), 4.38 (m, 2H, —NHCH(CH₂Ph) C(O)O—), 6.36 (d, ³J_{H—H}=9 Hz, 2H, —NHCH(CH(CH₃)₂)C(O)O—), 6.48 (d, ³J_{H—H}=9 Hz, 2H, —C(O)NHC(CH₂Ph)HC(O)—), 7.13-7.28 (m, 10H, —C₆H₅), 1.18, 1.24, 1.44, 1.51, 3.95-4.05 (all remaining protons) ppm. (See, FIG. 16)¹³C NMR (500 MHz, 303 K, DMSO-d₆): δ 17.36, 18.74, 24.77, 27.60, 28.27, 30.07, 40.00, 54.09, 57.56, 64.34, 126.45, 127.92, 128.99, 137.05, 156.52, 157.90, and 172.39 ppm. (M_{w}=105 kDa, Mₙ₌68 kDa, D, =1.5, T_{g}=36° C., T_{d}=287° C.). (See, FIG. 18)

Poly[(1-VAL-8)₉₀-co-(1-PHE-8)jo]. (10% PHE8 P(1-VAL-8)). ¹H NMR (300 MHz, 303 K, DMSO-d₆): δ=0.83 (m, 12H, —CH(CH₃)₂), 1.95 (m, 2H, —NHCH(CH(CH₃)₂)C(O)O—), 2.82-2.97 (m, 4H, —NHCH(CH₂Ph)C(O)O—), 4.38 (m, 2H, —NHCH(CH₂Ph) C(O)O—), 6.36 (d, ³J_{H—H}=9 Hz, 2H, —NHCH(CH(CH₃)₂)C(O)O—), 6.48 (d, ³J_{H—H}=9 Hz, 2H, —C(O)NHC(CH₂Ph)HC(O)—), 7.13-7.28 (m, 10H, —C₆H₅), 1.18, 1.24, 1.44, 1.51, 3.95-4.05 (all remaining protons) ppm. (See, FIG. 16). ¹³C NMR (500 MHz, 303 K, DMSO-d₆): δ 18.52, 19.55, 25.83, 28.20, 28.91, 31.04, 40.00, 54.60, 58.37, 64.69, 127.12, 128.87, 129.91, 137.20, 157.37, 158.00, and 173.00 ppm. (M_{w}=84 kDa, Mₙ=49 kDa, Ðₘ=1.7, T_{g}=34° C., T_{d}=297° C.). (See, FIG. 18).

### Example 14

### Uniaxial Mechanical Property Measurements

To compression mold PEU films, the polymers were blended into a fine powder using a StrandMill Grinder. Each polymer powder was funneled in a mold (5 cm×5 cm×0.5 mm) and then placed in a vacuum compression instrument (TMP Technical Products Corp). Each polymer was melted and allowed to equilibrate for 30 minutes followed by degassing cycles (1000 psi) to remove air-bubbles. The polymer molds were consecutively pressed at 69 MPa, 103 MPa, and 138 MPa. The mold was then rapidly cooled to afford the respective amorphous polymer films, which were then cut into tensile bars (4.76 mm×38.1 mm×0.5 mm). Elastic moduli, yield stress (σ_{y}), and yield strain (ε_{y}) were determined using tensile tests (Instron 5543 Universal Testing Machine) at 25° C. at a strain rate of 25.4 mm/min. The dimensions of each specimen were measured using calipers to ensure accurate measurement. The viscoelastic linear region was determined using linear regression at 10% strain. The yield stress and yield strain were subsequently measured after the linear region. Statistical analyses were done using a one-way ANOVA with Tukey post hoc analysis. A value of p<0.05 was considered significant. See Table 6, above.

### Example 15

### Burst-Test Mechanical Property Measurements. PEU-ECM Films

Burst-test mechanical properties were obtained by blade coating PEU solutions on polyethylene terephthalate (PET) substrates. PEU copolymer analogues were dissolved in acetone at 5% weight and then filtered with 5 micrometer syringe filters to remove impurities. The solutions were then concentrated to 33% weight polymer. Extracellular matrix (Cook Biotech SIS 2.0 1-LL) was secured to PET with tape on the edges. Polymer solutions were then blade coated (8 cm blade width, gap height 300 m) on ECM and allowed to air dry for 24 hours. The PEU-ECM films were then further dried under reduced pressure to remove residual solvent. PEU-ECM films were cut into 5×5 cm sheets and submerged in 1×PBS (pH=7.4) for five minutes. Films were then fastened in an ASTM D 3787-07 standard ball-burst apparatus and burst with a constant rate of traverse (25.4 mm/min) (FIGS. 27A-E). ASTM D 3787-07 (2007), is incorporated herein by reference in its entirety. Force at break, extension at break, and ultimate tensile strength were recorded until film failure along with the location of the failure. (See, FIGS. 28-31; Table 7, above) Statistical analyses were done using a one-way ANOVA with Tukey post hoc analysis. A value of p<0.05 was considered significant.

### Example 16

### PEU Free-Standing Films

PEU polymer free-standing films were prepared by blade coating with slight adaptations from the procedure described in Example 15, above. In short, PEU copolymers were dissolved in acetone at 35% weight. Polymer solutions were then blade coated (8 cm blade width, 400 m gap height) on PET and allowed to air dry for 24 hours. The PEU films were then further dried under reduced pressure to remove residual solvent. Films were then cut into 5×5 cm sheets and submerged in 1×PBS (pH=7.4) for five minutes. Films were then fastened in an ASTM D 3787-07 (2007) standard ball-burst apparatus and burst with a constant rate of traverse (25.4 mm/min) as set forth in Example 15, above. Force at break, extension at break, and relative stiffness were recorded until film failure along with the location of the failure. (See, FIGS. 27A-E; Table 8).Statistical analyses were done using a one-way ANOVA with Tukey post hoc analysis. A value of p<0.05 was considered significant.

### Example 17

### Film Water Uptake

Water uptake (WU) was carried out following previously published work. See, Gao, Y.; Childers, E. P.; Becker, M. L. Poly (ester urea) s for Vascular Tissue Engineering. 2015 DOI: 10.1021/acsbiomaterials.5b00168, the disclosure of which is incorporated herein by reference in its entirety. Samples prepared from blade-coating as described in Example 15, above (35% weight polymer) were weighed to determine an initial mass (WI). Samples were then placed in PBS (pH=7.4, r.t.) for one week. Samples were then removed, blotted dry with a paper towel and then immediately weighed to determine water uptake (W). The percentage of water uptake was calculated from equation 1.

$$WU ( \% ) = \frac{{W}_{t} - {W}_{i}}{{W}_{i}} \times 100 \% \qquad ( Eq . 1 )$$

The results are shown in FIG. 22, above.

In light of the foregoing, it should be appreciated that the present invention significantly advances the art by providing an amino acid-based poly(ester urea) polymer mesh for hernia and other soft tissue applications that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A polymer mesh for soft tissue repair comprising an amino acid-based poly(ester urea) polymer wherein the amino acid-based poly(ester urea) polymer is a copolymer comprising a first type of amino acid based polyester monomer residue and a second type amino acid based polyester monomer residue separated by urea bonds, wherein the first type of amino acid based polyester monomer residue and the second type of amino acid based polyester monomer residue have different chemical structures.

15. The polymer mesh for soft tissue repair of claim 14 wherein said first type of amino acid based polyester monomer residues comprises two amino acid residues separated by from about 2 to about 20 carbon atoms.

16. The polymer mesh for soft tissue repair of claim 15 wherein each of said two amino acids in said first type of amino acid based polyester monomer are selected from the group consisting of L-valine, L-leucine, L-isoleucine, L-serine, L-alanine, L-glycine, L-aspartic acid, L-asparagine, L-arginine, L-phenylalanine, L-methionine, benzyl protected L-tyrosine, and combinations thereof.

17. The polymer mesh for soft tissue repair of claim 14 wherein said first type of amino acid based polyester monomer residue comprises two valine residues separated by from about 2 to about 20 carbon atoms.

18. The polymer mesh for soft tissue repair of claim 14 wherein said first type of amino acid based polyester monomer residue comprises three or more valine residues, wherein each of said three or more valine residues is separated from the other valine residues by from about 2 to about 20 carbon atoms.

19. The polymer mesh for soft tissue repair of claim 14 wherein the second type of amino acid based polyester monomer residue comprises two amino acid residues separated by from about 2 to about 20 carbon atoms.

20. The polymer mesh for soft tissue repair of claim 14 wherein each of the two amino acid residues in said second type of amino acid based polyester monomer is selected from the group consisting of L-valine, L-leucine, L-isoleucine, L-serine, L-alanine, L-glycine, L-aspartic acid, L-asparagine, L-arginine, L-phenylalanine, L-methionine, benzyl protected L-tyrosine, and combinations thereof.

21. The polymer mesh for soft tissue repair of claim 14 wherein said second type of amino acid based polyester monomer comprises two phenylalanine residues separated by from about 2 to about 20 carbon atoms.

22. The polymer mesh for soft tissue repair of claim 14 wherein the molar ratio of said first amino acid based polyester monomer residue and a second amino acid based polyester monomer residue is from about 1:19 to about 19:1.

23. The polymer mesh for soft tissue repair of claim 14 wherein said first type of amino acid based polyester monomer residue comprises two valine residues separated by from about 2 to about 20 carbon atoms and said second type of amino acid based polyester monomer residue comprises two phenylalanine residues separated by from about 2 to about 20 carbon atoms.

24. The polymer mesh for soft tissue repair of claim 23 wherein said second type of amino acid based polyester monomer residue comprises from about 5 mole percent to about 30 mole percent of the amino acid-based poly(ester urea) polymer.

25. (canceled)

26. The polymer mesh for soft tissue repair of claim 1 wherein said the amino acid-based poly(ester urea) polymer has the formula: 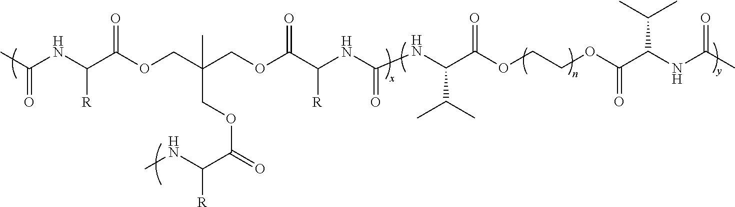
where n is an integer from 1 12; x is a mole fraction from about 0.001 to about 0.100; y is a mole fraction from about 0.900 to about 0.999; and each R is selected from the group consisting —CH₃, —(CH₂)₃NHC(NH₂)C═NH, —CH₂CONH₂, —CH₂COOH, —CH₂SH, —(CH₂)₂COOH, —(CH₂)₂CONH₂, —NH₂, —CH₂C═CH—N═CH—NH, —CH(CH₃)CH₂CH₃, —CH₂CH(CH₃)₂, —(CH₂)₄NH₂, —(CH₂)₂SCH₃, —CH₂Ph, —CH₂OH, —CH(OH)CH₃, —CH₂—C=CH—NH-Ph, —CH₂-Ph-OH, or —CH(CH₃)₂.

27. The polymer mesh for soft tissue repair of claim 14 wherein said the amino acid-based poly(ester urea) polymer has the formula: 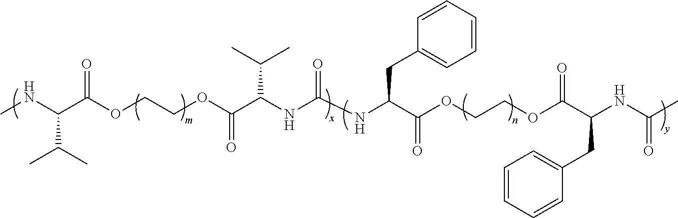
where n and m are each an integer from 1 to 12; x is a mole fraction from about 0.05 to about 0.95; y is a mole fraction from about 0.95 to about 0.05.

28. The polymer mesh for soft tissue repair of claim 27 wherein y is a mole fraction from about 0.05 to about 0.30.

29. (canceled)

30. The polymer mesh for soft tissue repair of claim 1 further comprising an extracellular matrix (ECM).

31. The polymer mesh for soft tissue repair of claim 30 wherein said extracellular matrix comprises 2.0 1-1 LL SIS-ECM, 2.0 4-LL SIS ECM, Blanket 2 LVP SIS ECM, or Blanket 4 LVP SIS ECM.

32. (canceled)

33. (canceled)

34. (canceled)

35. The polymer mesh for soft tissue repair of claim wherein said amino acid-based poly(ester urea) polymer has a glass transition temperature (T_{g}) of from about 28° C. to about 57° C. as measured by differential scanning calorimetry.

36. (canceled)

37. The polymer mesh for soft tissue repair of claim 14 wherein said amino acid-based poly(ester urea) polymer has a Young's modulus of from about 10 MPa to about 500 MPa as measured by uniaxial tensile testing.

38. The polymer mesh for soft tissue repair of claim 14 wherein said amino acid-based poly(ester urea) polymer has a yield stress (σ_{y}) of from about 2 MPa to about 100 MPa as measured by uniaxial tensile testing.

39. The polymer mesh for soft tissue repair of claim 14 wherein said amino acid-based poly(ester urea) polymer has a yield strain (ε_{y}) of from about 1% to about 50% as measured by uniaxial tensile testing.

40. The polymer mesh for soft tissue repair of claim 14 wherein said amino acid-based poly(ester urea) polymer has a force at break from about 50 N to about 500 N as measured by burst testing at a constant rate of traverse of 25.4 mm/min.

41. The polymer mesh for soft tissue repair of claim 14 wherein said amino acid-based poly(ester urea) polymer has an extension at break from about 0.5 cm to about 5 cm as measured by burst testing at a constant rate of traverse of 25.4 mm/min.

42. The polymer mesh for soft tissue repair of claim 14 wherein said amino acid-based poly(ester urea) polymer has a relative stiffness from about 25 N/cm to about 200 N/cm as measured by burst testing at a constant rate of traverse of 25.4 mm/min.

43. The polymer mesh for soft tissue repair of claim 14 wherein said amino acid-based poly(ester urea) polymer has a water uptake from about 0 mass % to about 45 mass % as measured by phosphine buffered saline solution (PBS) swelling/water uptake testing

44. The polymer mesh for soft tissue repair of claim 14 formed by compression molding, blade coating, or vacuum molding.

45. (canceled)

46. (canceled)

47. A method of forming the polymer mesh for soft tissue repair of claim 14 comprising:
A) forming an amino acid-based poly(ester urea) polymer comprising the residue of two or more amino acid based polyester monomers, wherein said one or more amino acid based polyester monomer residues each comprise two amino acid residues separated by from about 2 to about 20 carbon atoms and said two or more amino acid based polyester monomers are separated by urea bonds;
B) forming the amino acid-based polymer of step A into a 3-dimensional mesh.

48. (canceled)

49. The method of claim 47 wherein said one or more amino acid based polyester monomers comprises the residues of two valine molecules separated by from about 2 to about 20 carbon atoms.

50. (canceled)

51. (canceled)

52. (canceled)

53. The method of claim 47 wherein at least of said one or more amino acid based polyester monomers is a branched amino acid based polyester monomer.

54. (canceled)

55. The method of claim 47 wherein the step of forming the amino acid-based polymer of step A into a 3-dimensional mesh is performed by compression molding, vacuum molding, blade coating, flow coating, electrospinning or solvent casting.

56. (canceled)

57. A method of forming the polymer mesh for soft tissue repair of claim 1 comprising:
a) dissolving amino acid-based poly(ester urea) polymer in to a suitable solvent or solvent solution;
b) securing extracellular matrix (ECM) to a substrate to form a ECM/substrate combination that is configured for use in a blade coating, flow coating, or solvent casting device;
c) feeding the amino acid-based poly(ester urea) polymer solution of step (a) into a solution well that is configured for use in a blade coating, flow coating, or solvent casting device;
d) securing the solvent well from step (c) to a blade coating, flow coating, or solvent casting device and moving ECM/substrate combination through the blade coating, flow coating, or solvent casting device at a velocity from about 0 cm/s to about 200 cm/s to apply said amino acid-based poly(ester urea) polymer to said ECM/substrate combination with the ECM acting as the substrate for the amino acid-based poly(ester urea) polymer;
e) removing the solvent from the poly(ester urea) polymer coated extracellular ECM/substrate combination of step (d) by drying at a temperature of from about 20° C. to about 35° C. for a period of from about 1 hour to about 24 hours;
f) placing the poly(ester urea) polymer coated extracellular ECM/substrate combination from step (e) under a vacuum pressure of from about 5 mm/Hg to about 25 mm/Hg for from about 1 hour to about 24 hours to remove any residual solvent; and
g) removing the PEU/ECM composite from the substrate to provide the PEU/ECM composite mesh.
